(19) United States
(12) Patent Application Publication
(10) Pub. No.: US 2005/0118624 A1
(43) Pub. Date: Jun. 2, 2005

(54) Fluorescent probes for ribosomes and method of use

(75) Inventors: Zhenkun Ma, Dallas, TX (US); Jing Li, Dallas, TX (US); In Ho Kim, Irving, TX (US); Yafei Jin, Dallas, TX (US); Anthony Simon Lynch, Dallas, TX (US); Eric Roche, Carrollton, TX (US); Doug Beeman, Dallas, TX (US)

Correspondence Address: JACKSON WALKER LLP, 2435 NORTH CENTRAL EXPRESSWAY, SUITE 600, RICHARDSON, TX, 75080, US

(73) Assignee: Cumbre Inc., Dallas, TX (US)

(21) Appl. No.: 10/954,996

(22) Filed: Sep. 30, 2004

Related U.S. Application Data
(60) Provisional application No. 60/508,401, filed on Oct. 3, 2003.

(51) Int. Cl. C12Q 1/68; C07H 17/08; C07H 21/04

(52) U.S. Cl. 435/6; 536/24.1; 536/25.32; 544/229; 544/230; 536/7.1

(57) ABSTRACT

Fluorescent probes that have binding affinity to ribosomes. The fluorescent probes are useful tools for identifying small molecules that bind to the 50S or 30S subunits of the bacterial and other ribosomes and serve as novel ribosome inhibitors. These probes are also useful for determining the interactions between a specific ligand and the ribosome.

This application claims priority to U.S. Provisional Patent Application, Ser. No. 60/508,401, entitled “Fluorescent Probes for Ribosomes and Method of Use” filed on Oct. 3, 2003, the entire content of which is hereby incorporated by reference.

### BACKGROUND

The present invention is related to fluorescent probes having high binding affinity to ribosomes and their uses. The fluorescent probes of this invention are useful tools for identifying small molecules that bind to the 50S or 30S subunits of the bacterial ribosome and serve as novel ribosome inhibitors. These probes are also useful for determining the interactions between a specific ligand and the ribosome.

Antibiotics are commonly utilized to fight a variety of microbial infections. However, many clinically important strains of bacteria have become resistant to one or more classes of the available antibiotics. Novel antimicrobial agents with activity against these resistant organisms are needed for the effective management of resistant microbial infections. Although not wanting to be bound by theory, the bacterial ribosome is one of the most important targets for both naturally occurring and synthetic antibiotics. Consequently, the antibiotics that target the bacterial ribosome are used widely in clinical settings for the treatment of bacterial infections (Chopra, I, Expert Opinion of Investigational Drugs, 1998, 7, 1237-1244). Examples of naturally occurring antibiotics or their derivatives targeting the bacterial ribosome are the macrolide class, chloramphenicol, clindamycin, the tetracycline class, spectinomycin, streptomycin, the aminoglycoside class and amikacin. Currently, the oxazolidinone class is the only synthetic ribosome inhibitor used clinically. The binding sites of ribosome antibiotics are broadly distributed between the 30S and 50S subunits of the ribosome and these antibiotics exert their antibacterial effects by a variety of mechanisms. In addition, ribosome antibiotics exhibit low frequency of mutational resistance against various pathogenic bacteria. The proven druggability of the ribosome, the high number of available binding sites and the low frequency of mutational resistance make the bacterial ribosome an attractive target for the discovery of novel antibacterial agents.

Several relevant biochemical assays have been developed for identifying ribosome inhibitors. The most commonly used assay in this regard is a coupled transcription and translation assay using luciferase as the reporter system (Murray, R. W.; et al. Antimicrobial Agents and Chemotherapy, 2001, 45, 1900-1904). This particular assay is relatively crude and covers both RNA and protein synthesis pathways. The assay reveals no information about the binding sites of the inhibitors identified. A more precise biochemical assay is available that monitors the peptidyl transferase activity of the ribosome (Lynch, A. S., U.S. Pat. No. 5,962,244; Polacek, N., et al. Biochemistry, 2002, 41, 11602-11610). This assay monitors a single step of the protein synthesis process but is not informative about the binding sites of the inhibitors.

The current invention describes an array of novel fluorescent probes that bind the bacterial ribosome. These fluorescent probes are useful for the identification of novel ribosome ligands that competitively or allosterically replace the fluorescent probes bound to the bacterial ribosome. The fluorescent probes of the current invention cover various specific antibiotic binding sites of bacterial ribosomes and allow for the rapid identification of small molecule leads as potential starting points for the development of novel antimicrobial agents. In addition, this methodology provides important binding and mechanistic information that allows for rapid advancement of the initial leads through structure-based design and optimization. Multiple probes have been prepared and optimized for their ribosome binding affinity. The ligands identified by this assay interact with or disturb important drug binding sites and are likely to be effective and selective inhibitors of the ribosome. This assay format reduces the number of promiscuous hits due to aggregation or low solubility. The binding site information associated with the leads is immediately available and is useful for structure-based drug design and optimization.

Fluorescence polarization competition assays are utilized for the study of DNA-DNA, DNA-RNA, DNA-protein, RNA-protein, protein-protein, and small molecule-protein interactions. Fluorescence polarization competition assays are also used for screening small molecules that inhibit ligand-receptor interactions (Huang, X. J. Biomolecular Screening, 2003, 8, 34-38. Also see Panvera Fluorescence Polarization Guide, Third Edition, and references therein).

A fluorescent probe based on pleuromutilin is reported for screening of ribosome ligands of that specific binding site (Turconi, S.; et al. J. Biomolecular Screening, 2001, 6, 275-290; Hunt, E. Drugs of the Future, 2000, 25, 1163-1168). The screening was done at low compound concentration (10 μM, detecting only molecules with binding constants <4 μM) and in 1% DMSO limiting the solubility of detectable compounds.

Aminoglycoside-based fluorescent probes are prepared to study the binding between aminoglycosides and RNA molecules rather than the ribosome itself (Rando, R. R., et al, Biochemistry, 1996, 35, 12338-12346; Biochemistry, 1997, 36, 768-779; Bioorganic and Medicinal Chemistry Letters, 2002, 12, 2241-2244).

A fluorescent puromycin compound is prepared and applied for the synthesis of fluorescently labeled proteins, but not for screening of ribosome inhibitors (Doi, N., Genome Research, 2002, 487-492; Nemoto, N., FEBS, 1999, 462, 43-46).

A series of oxazolidinone photoaffinity probes that contains a photo reactive group rather than a fluorescent group in the molecule is reported in a PCT publication SN WO 02/56013 A2 and used to detect the binding site of oxazolidinones and used for identifying compounds that inhibit binding of oxazolidinone probes. The entire content of the PCT publication SN WO 02/56013 A2 entitled “Oxaxolidinone photoaffinity probes, uses and compounds” that was published on Jul. 18, 2002 having Colca, et al., listed as inventors is hereby incorporated as reference.

The fluorescent probes of this invention are structurally distinct and cover a broad range of drug binding sites that allow a systematic screening of various inhibitors of ribosome function.

### SUMMARY OF THE INVENTION

The current invention relates a series of fluorescent probes that reversibly bind to specific antibiotic binding sites of ribosomes and the use of these probes for the identification of small molecules that displace the fluorescent probes and for the study of specific ligand-ribosome interactions.

In one aspect, a series of fluorescent probes that reversibly bind to bacterial ribosomes are provided. The probes consist of a known ribosome ligand and a fluorophore connected through a linker. The ligand is any molecule known to bind to bacterial ribosomes in a reversible fashion. The fluorophore is a molecule that emits fluorescent light upon excitation. The linker is a chemical group between 2 and 16 atoms in length that links the ribosome ligand at one end and the fluorophore at another.

In a preferred embodiment, the ribosome ligand is a known antibiotic selected from a 14-membered ring macrolide, a 15-membered ring macrolide, a 16-membered ring macrolide, a tetracycline, an aminoglycoside, an oxazolidinone, clindamycin, puromycin, chloramphenicol, spectinomycin, streptomycin, amikacin and a pleuromutilin. The fluorophore is a molecule that emits fluorescent light upon excitation. The linker is a chemical group between 2 and 16 atoms in length that links the ribosome ligand at one end and the fluorophore at another.

In a more preferred embodiment, the ribosome ligand is a member of the macrolide family of antibiotics. Examples of macrolide antibiotics are erythromycin, erythromycylamine, clarithromycin, azithromycin, roxithromycin, dirithromycin, flurithromycin, oleandomycin, telithromycin, cethromycin, leucomycin, spiramycin, tylosin, rokitamycin, miokamycin, josamycin, and midecamycin. The linker is a 0 to 16-carbon chain optionally interrupted by 1 to 6 heteroatoms, functional groups, carbocycles and heterocycles. The fluorophore is selected from groups consisting of BODIPY, fluorescein, rhodamine, and dipyranone.

In another aspect, the fluorescent probes are used for high-throughput screening to identify small molecules that interact with ribosomes and for mechanistic studies of ligand-ribosome interactions. The methods described in this invention are generally applicable for the identification of compounds that selectively modulate the function of ribosomes derived or purified from any organism, and can therefore be applied toward the discovery of novel agents for controlling infections mediated by bacterial, fungal and protozoal organisms. Examples of bacterial organisms that may be controlled by the compositions resulting from the application of the methods of this invention include, but are not limited to the following organisms: Streptococcus pneumoniae, Streptococcus pyogenes, Enterococcus fecalis, Enterococcus faecium, Klebsiella pneumoniae, Enterobacter sps., Proteus sps., Pseudomonas aeruginosa, E. coli, Serratia marcesens, S. aureus, Coag. Neg. Staph., Acinetobacter sps., Salmonella sps, Shigella sps., Helicobacter pylori, Mycobacterium tuberculosis, Mycobacterium avium Mycobacterium intracellulare, Mycobacterium fortuitum, Mycobacterium chelonae, Mycobacterium kansasii, Haemophilus influenzae, Stenotrophomonas maltophilia, and Streptococcus agalactiae. The compositions and methods will therefore be useful for controlling, treating or reducing the advancement, severity or effects of nosocomial or non-nosocomial infections. Examples of nosocomial infection uses include, but are not limited to, urinary tract infections, pneumonia, surgical wound infections, bone and joint infections, and bloodstream infections. Examples of non-nosocomial uses include but are not limited to urinary tract infections, pneumonia, prostatitis, skin and soft tissue infections, bone and joint infections, intra-abdominal infections, meningitis, brain abscess, infectious diarrhea and gastrointestinal infections, surgical prophylaxis, and therapy for febrile neutropenic patients. The term “non-nosocomial infections” is also referred to as community acquired infections. None of the information provided herein is admitted to be prior art to the present invention, but is provided only to aid the understanding of the reader.

### BRIEF DESCRIPTION OF THE DRAWINGS

FIG. 1 shows examples of linkers, wherein the antibiotic is linked to the right-hand terminus of the linker and the Fluorophore is linked to the left-hand terminus of the linker;

FIG. 2 shows examples of nucleophile-reactive fluorophors;

FIG. 3 shows Scheme A, wherein an oxazolidinone core compound is reacted with an amine-reactive fluorophore catalyzed by an organic or inorganic base;

FIG. 4 shows examples of individual groups for A of structural formula I or II in FIG. 3;

FIG. 5 shows a specific example wherein an oxazolidinone core compound is reacted with an amine-reactive fluorophore under given reaction conditions;

FIG. 6 shows a specific example wherein an oxazolidinone core compound is reacted with an amine-reactive fluorophore under given reaction conditions;

FIG. 7 shows a specific example wherein an oxazolidinone core compound is reacted with an amine-reactive fluorophore under given reaction conditions;

FIG. 8 shows a specific example wherein an oxazolidinone core compound is reacted with an amine-reactive fluorophore under given reaction conditions;

FIG. 9 shows a specific example wherein an oxazolidinone core compound is reacted with an amine-reactive fluorophore under given reaction conditions;

FIG. 10 shows Scheme B, wherein a nucleophilic macrolide (“M”) having chemical structure III reacts with an amine-reactive fluorophore agent, in the presence or absence of a base, in an aprotic or protic solvent, to give fluorescent probe IV;

FIG. 11 shows examples of eleven nucleophilic macrolides;

FIG. 12 shows a specific example wherein a nucleophilic macrolide (“M”) having chemical structure III reacts with an amine-reactive fluorophore agent under given conditions;

FIG. 13 shows a specific example wherein a nucleophilic macrolide (“M”) having chemical structure III reacts with an amine-reactive fluorophore agent under given conditions;

FIG. 14 shows a specific example wherein a nucleophilic macrolide (“M”) having chemical structure III reacts with an amine-reactive fluorophore agent under given conditions;

FIG. 15 shows a specific example wherein a nucleophilic macrolide (“M”) having chemical structure III reacts with an amine-reactive fluorophore agent under given conditions;

FIG. 16 shows Scheme C, wherein the syntheses of specific macrolide probes are illustrated;

FIG. 17 shows Scheme D, wherein the syntheses of specific puromycin probes are illustrated;

FIG. 18 shows Scheme D, wherein a puromycin having chemical structure V reacts with a fluorophore to yield specific probes having chemical structure VI;

FIG. 19 shows Scheme D, wherein a puromycin having chemical structure VII reacts with a fluorophore to yield specific probes having chemical structure VIII;

FIG. 20 shows Scheme E, wherein an aminoglycoside having chemical structure X reacts with a fluorophore to yield specific probes having chemical structure XI;

FIG. 21 shows Scheme E, wherein an aminoglycoside having chemical structure X reacts with a fluorophore to yield specific probes;

FIG. 22 shows Scheme E, wherein an aminoglycoside having chemical structure X reacts with a fluorophore to yield specific probes;

FIG. 23 shows Scheme E, wherein an aminoglycoside having chemical structure X reacts with a fluorophore to yield specific probes;

FIG. 24 shows Scheme E, wherein an aminoglycoside having chemical structure X reacts with a fluorophore to yield specific probes;

FIG. 25 shows Scheme F, wherein a tetracycline reacts with a fluorophore to yield specific probes;

FIG. 26 shows Scheme F, wherein a tetracycline reacts with a fluorophore to yield specific probes;

FIG. 27 illustrates the synthesis to prepare the oxazolidinone core compound 112;

FIG. 28 illustrates the synthesis comprising compound 112 being reacted with different activated fluorophors to give a variety of oxazolidinone probes under typical coupling conditions;

FIG. 29 illustrates the synthesis of macrolide based probes;

FIG. 30 shows the synthesis of macrolide based probes;

FIG. 31 shows the synthesis of macrolide based probes;

FIG. 32 shows the synthesis of puromycin based probes;

FIG. 33 shows the structures of aminoglycoside based probes;

FIG. 34 shows the synthesis of tetracycline based probes;

FIG. 35 shows a graphic representation of the mP shift of a ribosome titration over time;

FIG. 36 shows a graphic representation of the mP shift due to competition with the Bodipy-FL erythromycin probe by the parent unlabeled erythromycin compound over time;

FIG. 37 shows a graphic representation of the mP shift due to competition with the Bodipy-FL erythromycin probe by other antibiotics;

FIG. 38 shows a graphic representation of effects of buffer composition on mP shift.

FIG. 39 shows the summarized kinetics values for Probe 203, Probe 238, and Probe 242.

### DETAILED DESCRIPTION OF THE INVENTION

One aspect of the current invention is related to fluorescent compounds that bind to a specific binding site of the bacterial ribosome. Another aspect of the current invention comprises methods for identifying ribosome ligands or inhibitors. One can also use this invention to study the binding, interaction, and mechanism of action of the ribosome ligands or ribosome inhibitors. Various terms used throughout this document have the meaning that would be attributed to those words by one skilled in the art.

The fluorescent compounds featured in this invention consist of two portions, the ribosome ligand portion that is responsible for binding to the specific binding site of the ribosome and the fluorophore portion that is responsible for giving a fluorescent signal when excited by light. The ligand portion could be based on any known ribosome ligands or inhibitors with known or undefined binding sites. The binding sites could be either on the 30S subunit or the 50S subunit and consist of ribosomal proteins, ribosomal RNAs or both of proteins and RNAs. The ribosome ligands could be either procaryotic ribosome selective or non-selective. Examples of selective ribosome ligands or inhibitors are erythromycin, erythromycylamine, clarithromycin, azithromycin, roxithromycin, dirithromycin, flurithromycin, oleandomycin, telithromycin, cethromycin, leucomycin, spiramycin, tylosin, rokitamycin, miokamycin, josamycin, midecamycin, virginiamycin, griseoviridin, chloramphenicol, clindamycin, linezolid, spectinomycin, chlortetracycline, oxytetracycline, demeclocycline, methacycline, doxycycline, minocycline, quinupristin, dalfopristin, streptomycin, amikacin, gentamicin, tobramycin, kanamycin, paromomycin, pleuromutilin, tiamulin, valnemulin, negamycin, viomycin, avilamycin, althiomycin, etc. Examples of non-selective ribosome ligands are puromycin, amicetin, blasticidin, gougerotin, sparsomycin, anisomycin, anthelmycin, bruceantin, narciclasine, pactamycin, purpuromycin, etc. The binding sites for many of the ribosome ligands or inhibitors have been defined by using biochemical, genetic and crystallographic techniques (The Ribosome: Structure, Function, Antibiotics, and Cellular Interactions, Garrett, R. A., et al. Ed. ASM Press: Washington, D.C., 2000). High resolution co-crystal structures for many of the ribosome inhibitors are available. In these cases, the precise binding sites of the inhibitors, the detailed interactions between inhibitors and ribosome are defined. Examples of inhibitors with available co-crystal structures are paromomycin, streptomycin, spectinomycin, chloramphenicol, clindamycin, puromycin, erythromycin A, clarithromycin, roxithromycin, cethromycin, tylosin, carbomycin A, spiramycin, azithromycin, tetracycline, edeine, pactamycin, hygromycin B, etc.

A fluorophore portion could be any structure that emits fluorescent light upon excitation. Examples of fluorophores are fluorescein, BODIPY, rhodamine, dipyrrinone, etc. (See Molecular Probes: Haugland, R. P., Handbook of Fluorescent Probes and Research Products, Molecular Probes, 9th Edition).

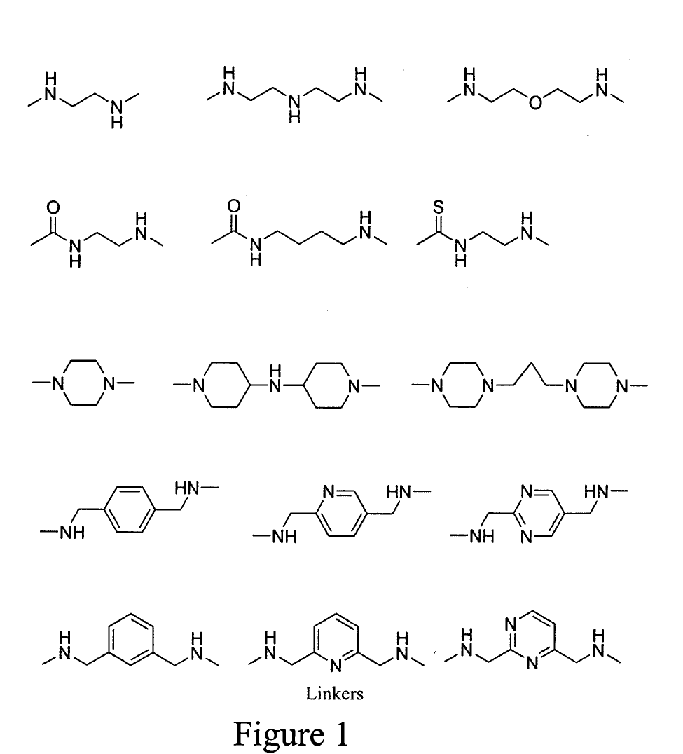

The ribosome ligand portion and the fluorophore portion are tethered by a linker group. The linker could have variable length and rigidity. It could contain any number of heteroatoms and or functional groups. It could contain any number of cyclic and or heterocyclic structures. Examples of linkers are shown in FIG. 1.

The fluorophore could be linked to various positions of the ligand molecules that could tolerate a large substituent. The linking points are selected by one skilled in the art based on known structure-activity relationships and if available, the co-crystal structural information.

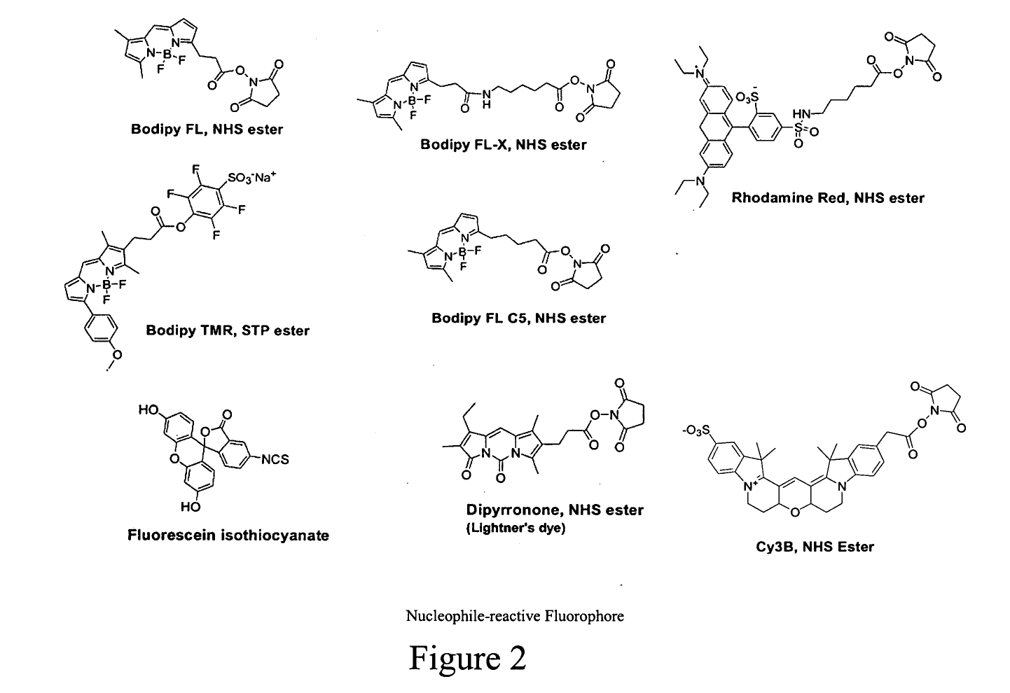

The compounds of this invention can be synthesized through chemical reactions known by those skilled in the art. Ribosome ligands with a nucleophilic group such as amino, hydroxyl or thiol can directly couple with a nucleophile-reactive fluorophore such as isothiocyanate, succinimidyl ester, STP ester, sulfonyl chloride, alkyl halide, maleimide, disulfide, etc. Optionally, a ligand can be first attached to a linker group and the combined molecule is then coupled with a fluorophore molecule; or the fluorophore can be attached to a linker group first and the combined molecule then reacts with the ligand. Examples of nucleophile-reactive fluorophore agents are shown in FIG. 2.

The following synthetic procedures are for illustration purposes. Probes of this invention can be prepared through other routes by one skilled in the art. Operations involving moisture and/or oxygen sensitive materials are conducted under an atmosphere of nitrogen. Unless noted otherwise, starting materials and solvents are obtained from commercially available sources and used without further purification. Flash chromatography is performed using silica gel 60 as absorbent. Thin layer chromatography (“TLC”) and preparative thin layer chromatography (“PTLC”) are performed using pre-coated plates purchased from E. Merck and spots are visualized with long-wave ultraviolet light followed by an appropriate staining reagent. Nuclear magnetic resonance (“NMR”) spectra are recorded on a Varian 400 MHz magnetic resonance spectrometer. ¹H NMR chemical shift are given in parts-per million (δ) downfield using the residual solvent signal (CHCl₃ =δ 7.27, CH₃OH=δ 3.31) as internal standard. ¹H NMR information is tabulated in the following format: number of protons, multiplicity (s, singlet; d, doublet; t, triplet; q, quartet; m, multiplet; td, triplet of doublet; dt, doublet of triplet), coupling constant (s) (J) in hertz. The prefix app is occasionally applied in cases where the true signal multiplicity is unresolved and prefix br indicates a broad signal. Electrospray ionization mass spectra are recorded on a Finnegan LCQ advantage spectrometer.

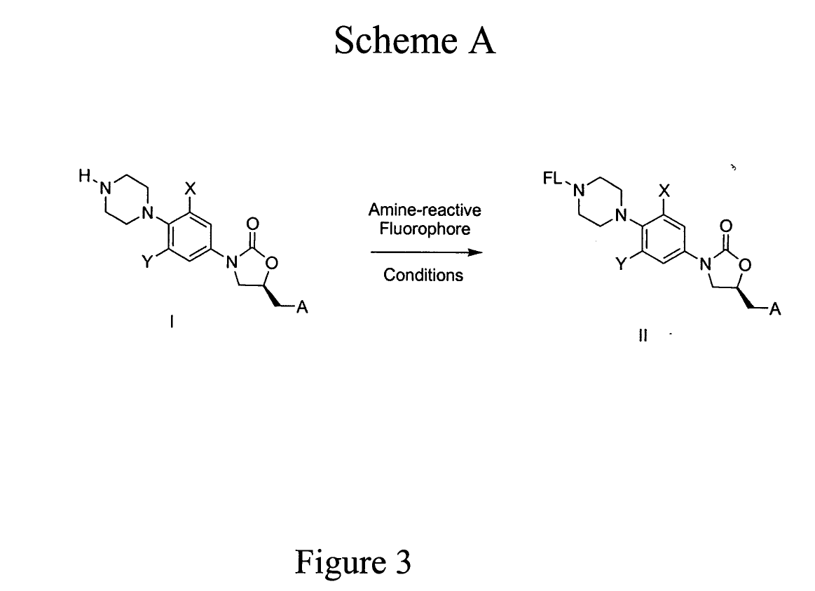

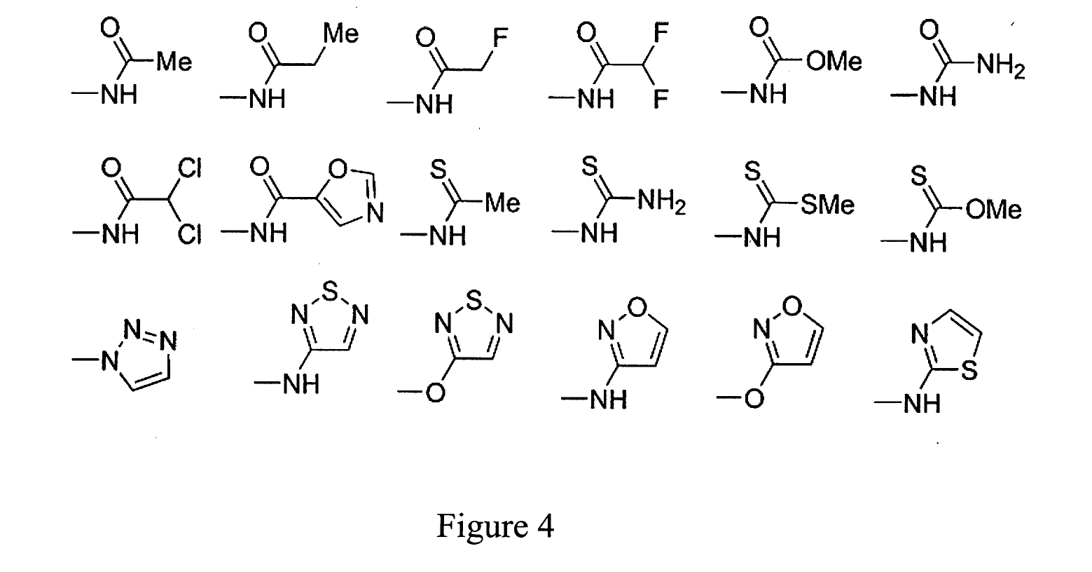

One series of fluorescent probes of this invention are based on the oxazolidinone class of antibiotics. All known oxazolidinones can be utilized for the preparation of ribosome probes. As illustrated by Scheme A in FIG. 3, oxazolidinone core I can react with 0.1 to 2.0 equivalents of an amine-reactive fluorophore catalyzed by a organic or inorganic base such as sodium carbonate, potassium carbonate, sodium hydroxide, triethylamine, pyridine; in a protic or aprotic solvent or solvent combination selected from DMF, DMSO, tetrahydrofuran, acetone, acetonitrile, ethanol and water; at a temperature ranging from −10° C. to 100° C. The groups X and Y are independently selected from hydrogen or fluorine atoms, and A comprise groups having structures as shown in FIG. 4.

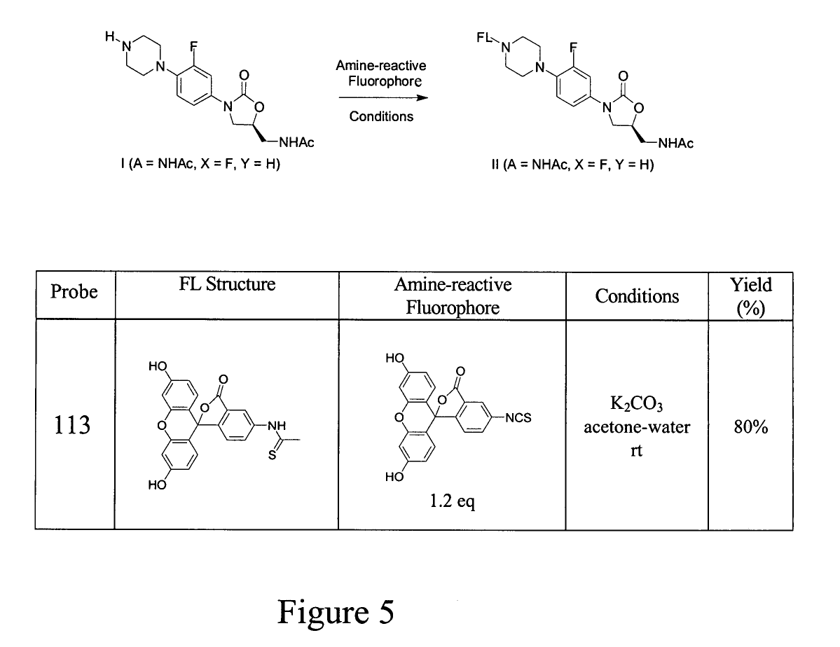

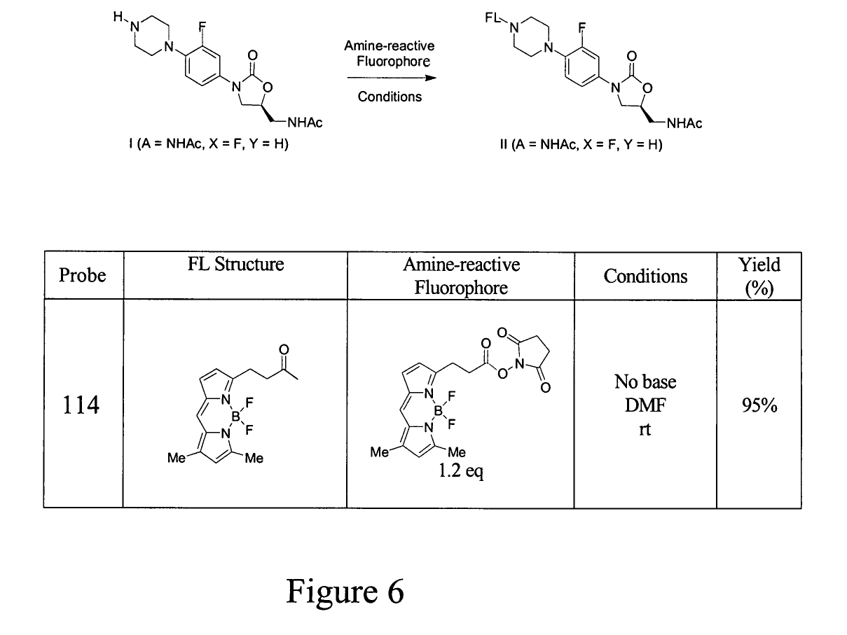

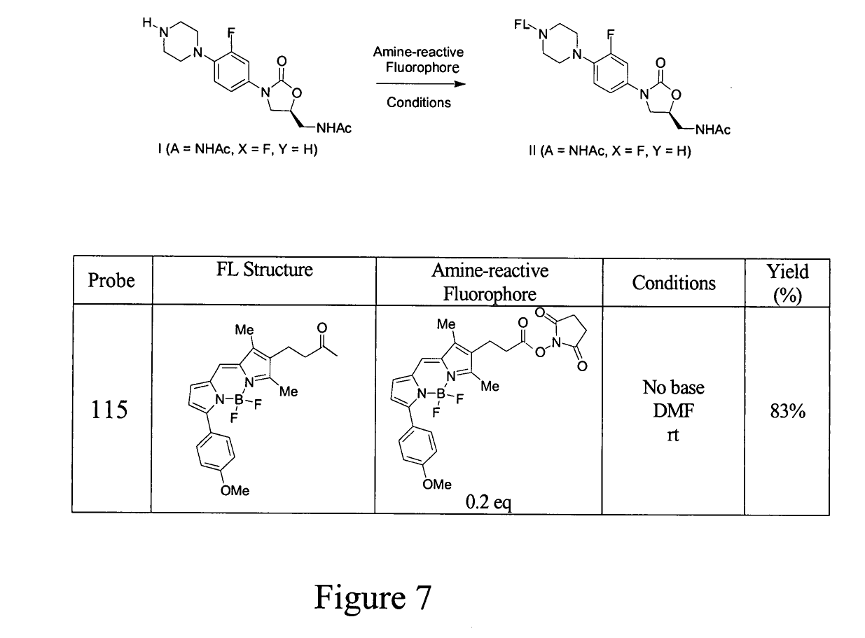

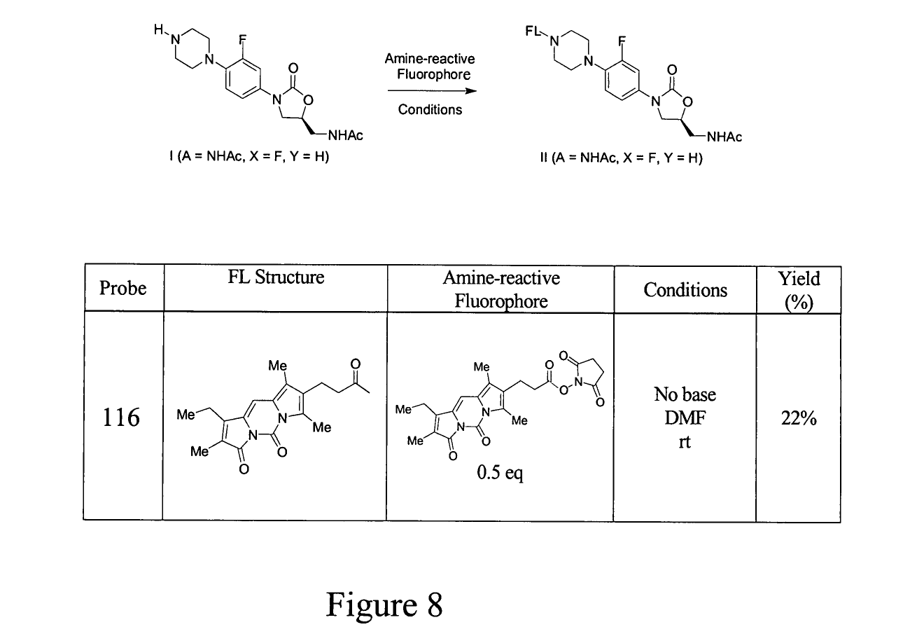

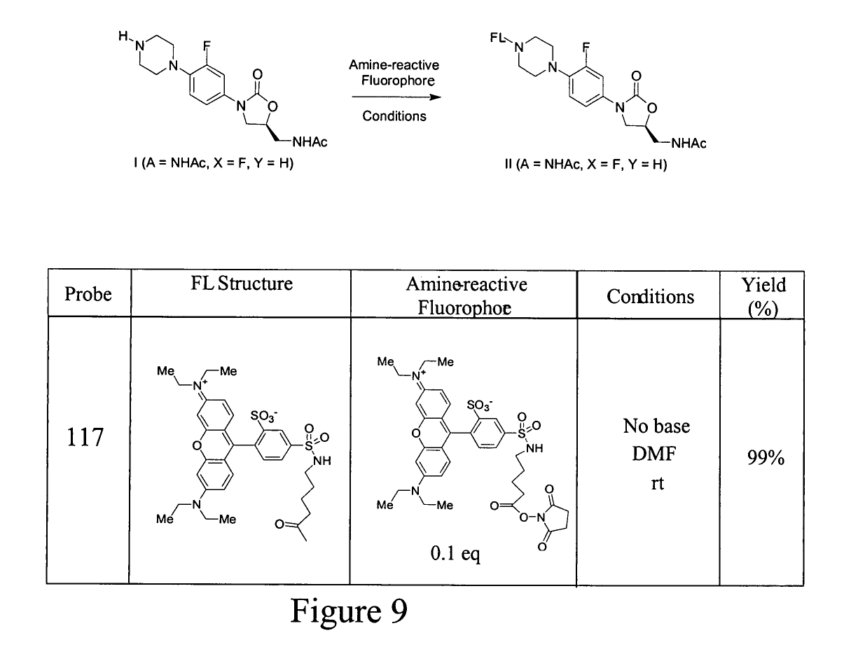

More specific examples include oxazolidinone core I, wherein X is a fluorine, Y is a hydrogen, and A is —NHAc, being prepared according to a literature procedure (Brickner, S. J., J. Med. Chem. 1996, 39, 673). Probes 113-117 illustrate how compound I is coupled with an amine-reactive fluorophore selected from Fluorescein isothiocyanate (FIG. 5), Bodipy FL SE (FIG. 6), Bodipy TMR STP ester (FIG. 7), Dipyrrinone SE (FIG. 8), and Rhodamine Red SE (FIG. 9), to give the desired probes.

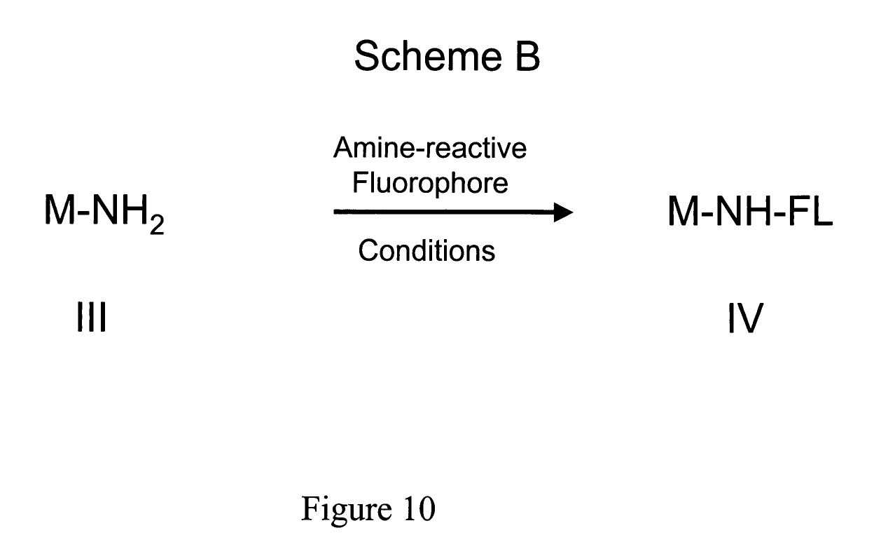

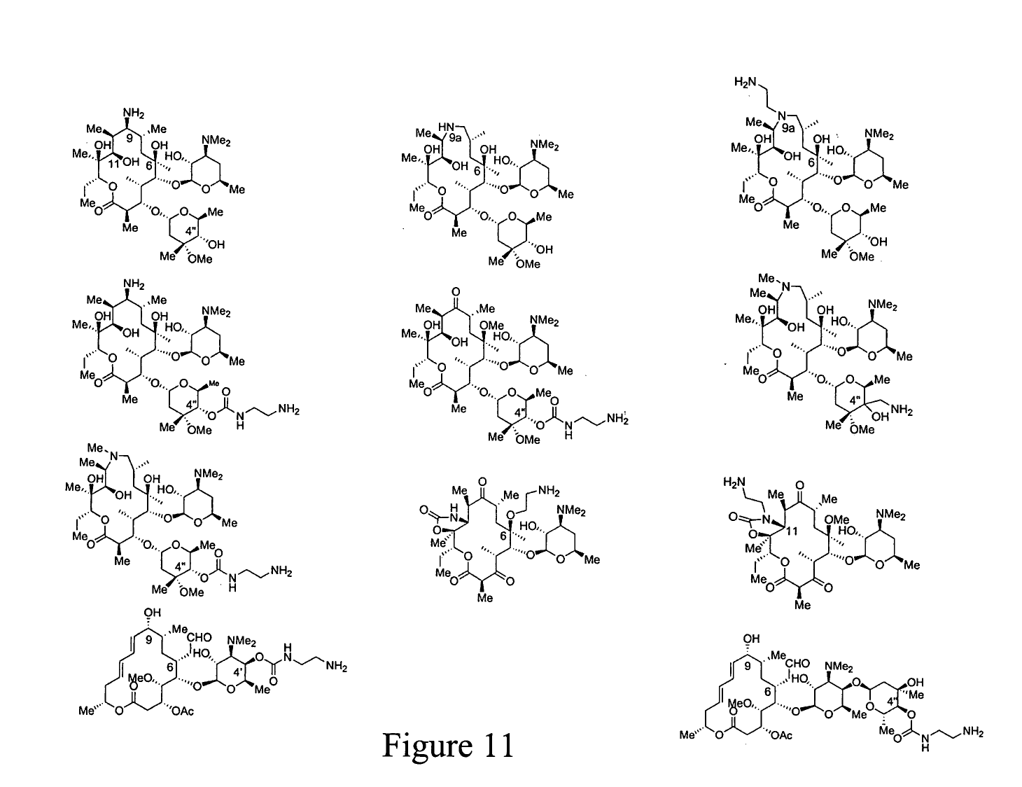

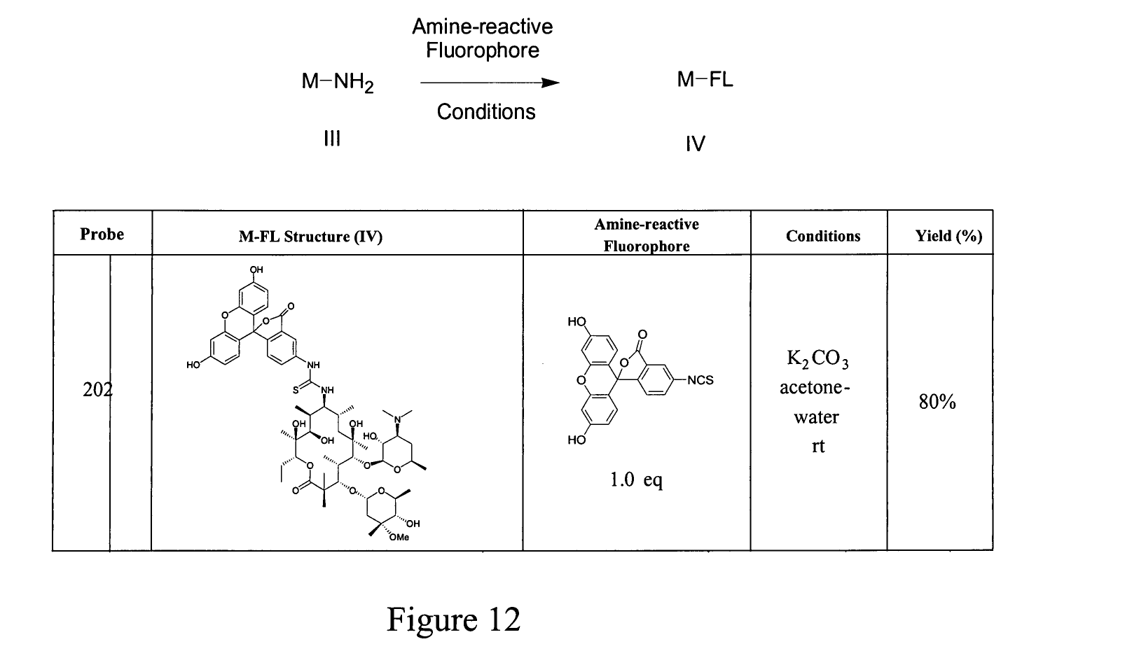

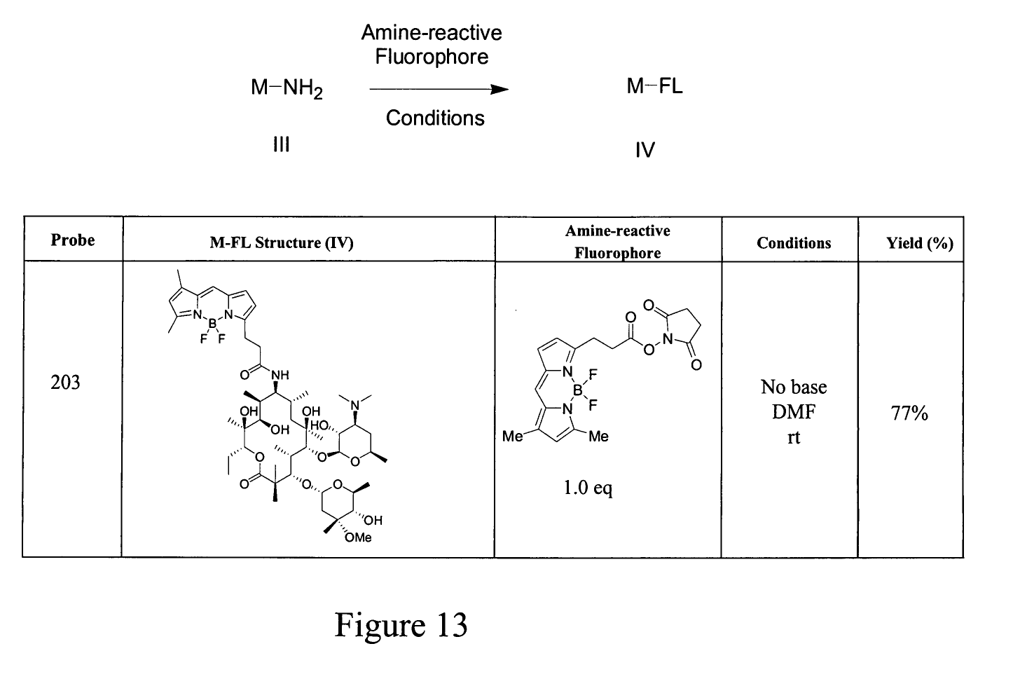

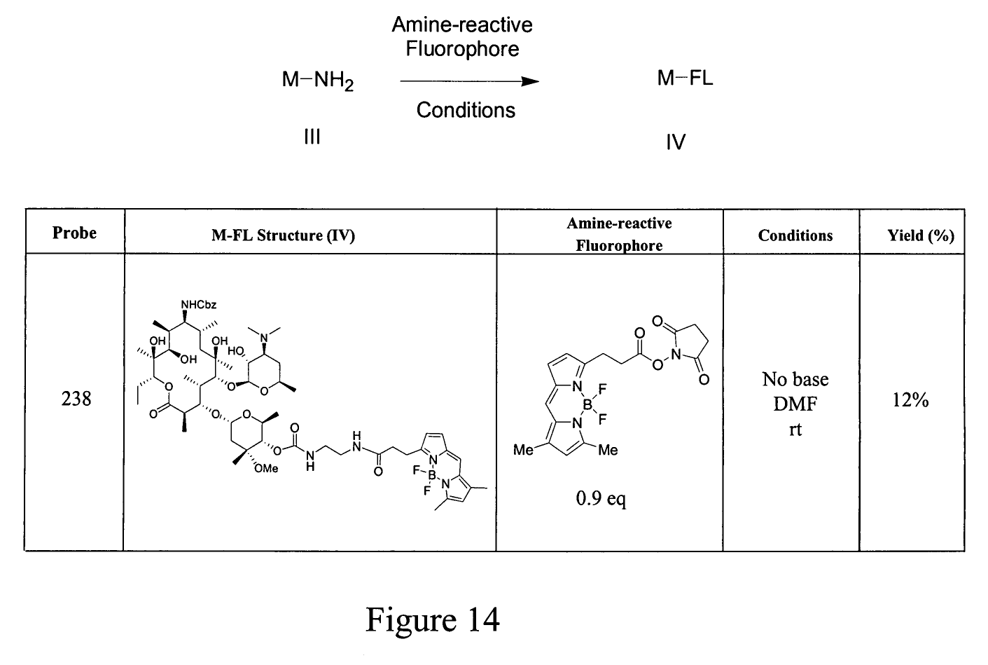

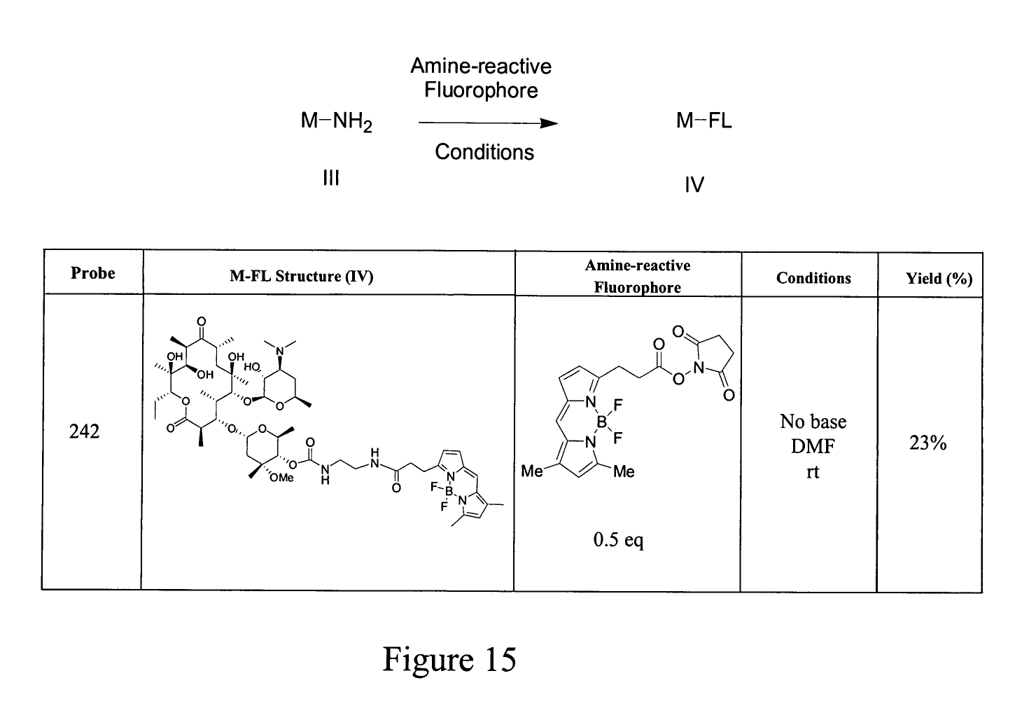

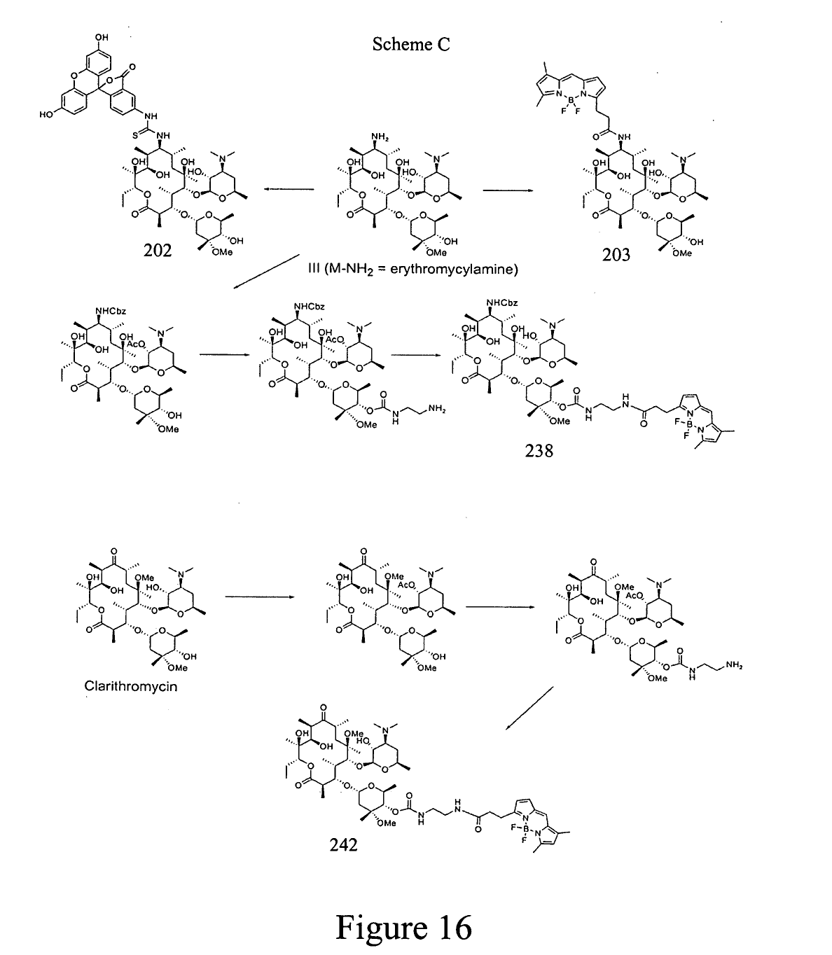

Another series of probes is based on the macrolide class of ribosome ligands. All known 14-membered ring, 15-membered ring and 16-membered ring macrolides can be utilized to prepare fluorescent probes. Examples of macrolides are erythromycin, erythromycylamine, clarithromycin, azithromycin, roxithromycin, dirithromycin, flurithromycin, oleandomycin, telithromycin, cethromycin, leucomycin, spiramycin, tylosin, rokitamycin, miokamycin, josamycin, and midecamycin. The fluorophores can be linked to a number of positions on macrolides. The preferred linking points are the 6-position, the 9-position, the 11-position and the 4″-position. In most cases, these positions need to be modified to introduce a nucleophilic group such as amine and thiol. Such modifications can be performed by one skilled in the art by following published procedures (see: Current Medicinal Chemistry, Anti-Infective Agents, 2002, 1, 15-34 for references). The nucleophilic macrolide (“M”) III can react with 0.1 to 2 equivalents of an amine-reactive fluorophore agent, in the presence or absence of a base, in an aprotic or protic solvent, to give fluorescence probe IV, as shown in Scheme B of FIG. 10, which is for illustration purposes only. Examples of eleven nucleophilic macrolides are shown in FIG. 11. Fluorescein isothiocyanate, Bodipy FL SE, Bodipy TMR STP ester, Dipyrrinone SE, and Rhodamine Red SE are examples of amine-reactive fluorophores. Examples of bases that can be utilized are sodium carbonate, potassium carbonate, sodium hydroxide, triethylamine, pyridine, DMAP and lutidine. Additionally, solvents such as DMF, DMSO, tetrahydrofuran, acetone, acetonitrile, ethanol and water can be utilized. Each of the macrolide based probes shown in the following examples are for illustration purposes only, and not intended to limit the scope of the invention. More specifically, compound III (when M-NH₂ is erythromycylamine) reacts with 5-fluorescein isothiocyanate at room temperature in acetone-water mixture, catalyzed by potassium carbonate to give 9-erythromycin-fluorescein probe, as shown in FIG. 12—Probe 202. Erythromycylamine also reacts with BODIPY FL OSu in DMF at room temperature to give 9-erythromycin-BODIPY FL probe, as shown in FIG. 13—Probe 203. Optionally, the 9-amino group of erythromycylamine can be protected by CBZ protecting group. The protected compound can then be reacted with CDI to form the 4″-acylimidazole intermediate. Reaction of the acylimidazole compound with ethylenediamine provides an intermediate with an amino group available for coupling with an amine-reactive fluorophore. Coupling of this intermediate with BODIPY FL OSu provides 4″-erythromycin-BODIPY FL probe, as shown in FIG. 14—Probe 238. Similarly, clarithromycin can be used as another macrolide core. Following the same process for making Probe 238, the 4″-clarithromycin-BODIPY FL probe is prepared, as shown in FIG. 15—Probe 242. Additionally, Scheme C in FIG. 16 shows the synthesis of Probes 202, 203, 238, and 242.

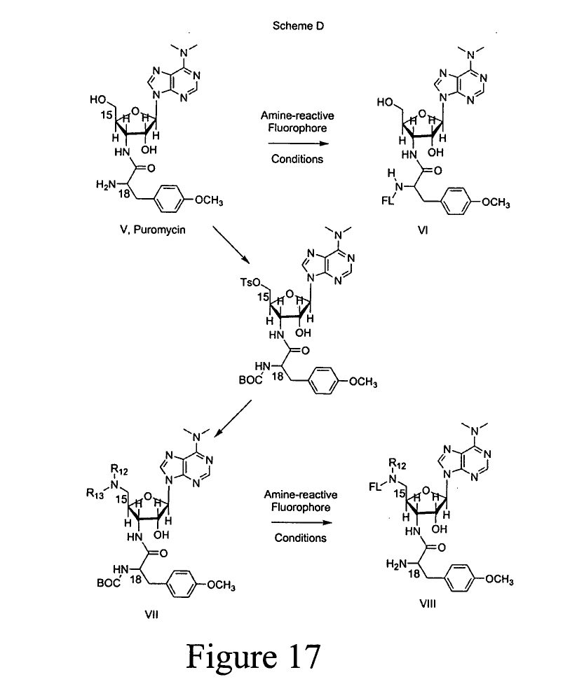

Fluorescent probes based on puromycin can be synthesized directly by coupling puromycin and an amine-reactive fluorophore as illustrated by Scheme D in FIG. 17. Reaction of puromycin (V) and 0.1 to 2.0 equivalents of an amine-reactive fluorophore in a solvent, in the presence or absence of a base, affords the desired puromycin fluorescent probe VI with a fluorophore linked to the 18-position. The typical solvent suitable for this reaction is DMF, NMP, DMSO, acetone, acetonitrile, THF, methylene chloride, ethanol or water. The typical base is sodium carbonate, potassium carbonate, sodium hydroxide, triethylamine, pyridine, DMAP or lutidine.

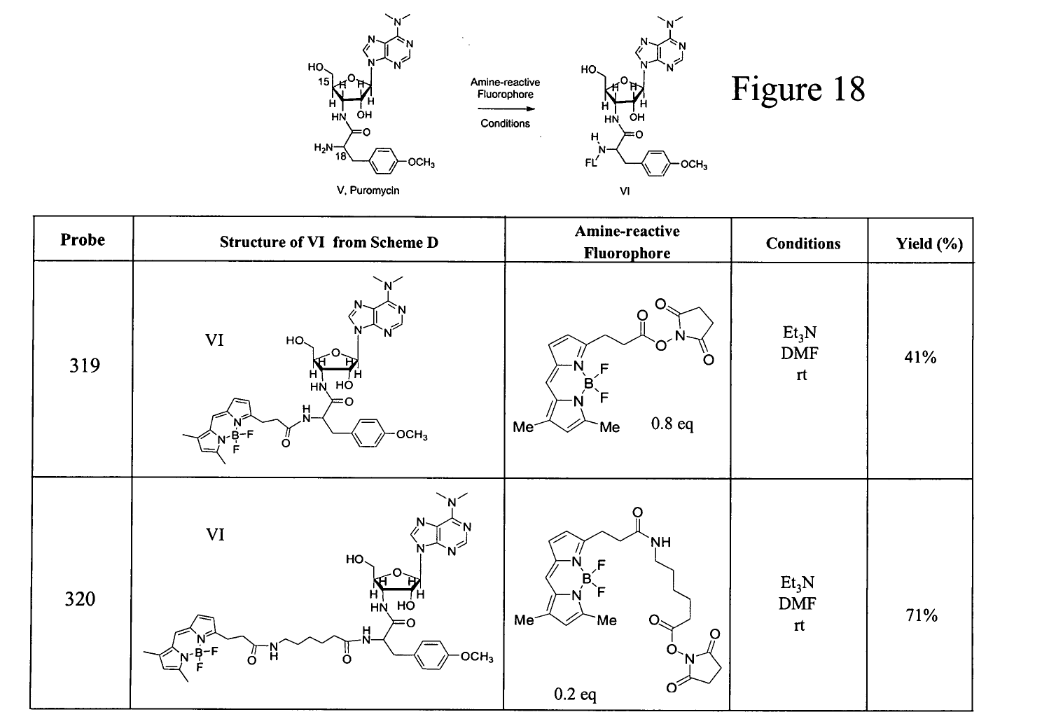

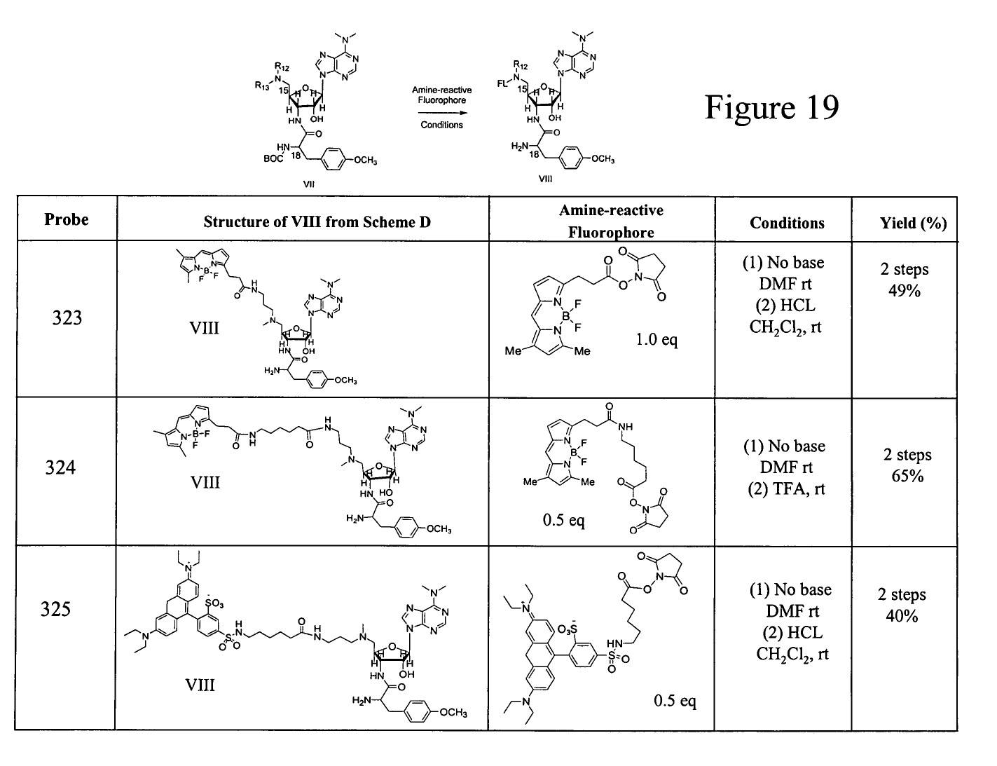

Fluorophore can be linked to the 15-position of puromycin through the BOC protected amine VII. VII is prepared from puromycin by first protecting the 18-amino group followed by converting the 15-hydroxy group to its tosylate. Nucleophilic substitution of the tosylate with an amine or diamine provides VII. Coupling of VII and 0.1 to 2.0 equivalents of an amine-reactive fluorophore under the typical coupling conditions provided the BOC protected puromycin fluorescent probes. Deprotection of the BOC protecting group under typical conditions for removing a BOC protecting group provides the desired fluorescent probes VIII (T. W. Greene and P. G. M. Wuts, Protective Groups in Organic Synthesis, 3^{rd} Ed.). The preparation of 15-substituted puromycin fluorescent probes is illustrated in Scheme D. More specifically, when R₁₂ is a methyl and R₁₃ is an aminoethyl group, the amino group reacts with 0.1 to 2.0 equivalents of an amine-reactive fluorophore under the typical coupling conditions provided the BOC protected puromycin fluorescent probes. Removal of the BOC protecting group under typical conditions provides the desired fluorescent probes VIII (R₁₂=Me). Examples of puromycin-based probes are illustrated in Probes 319-320 in FIG. 18 and Probes 323-325 in FIG. 19.

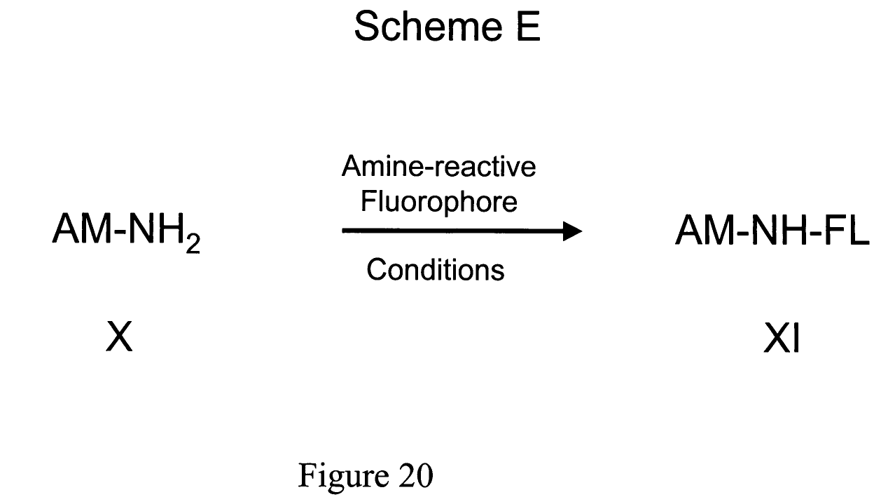

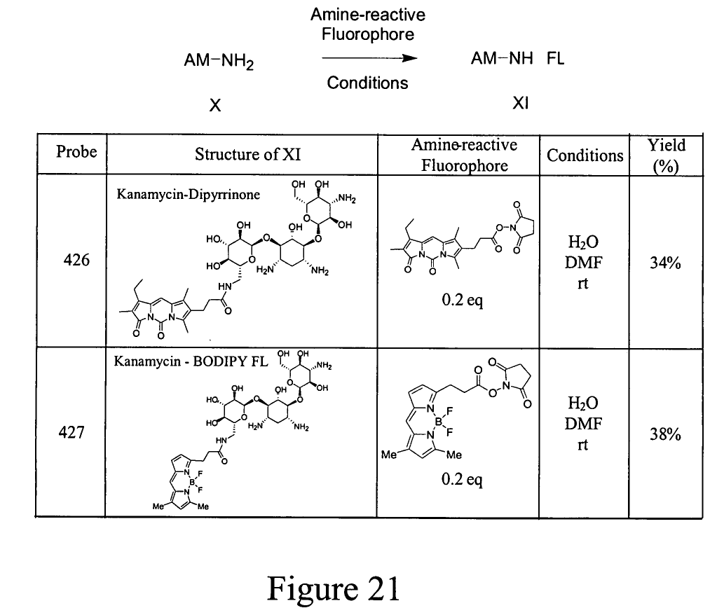

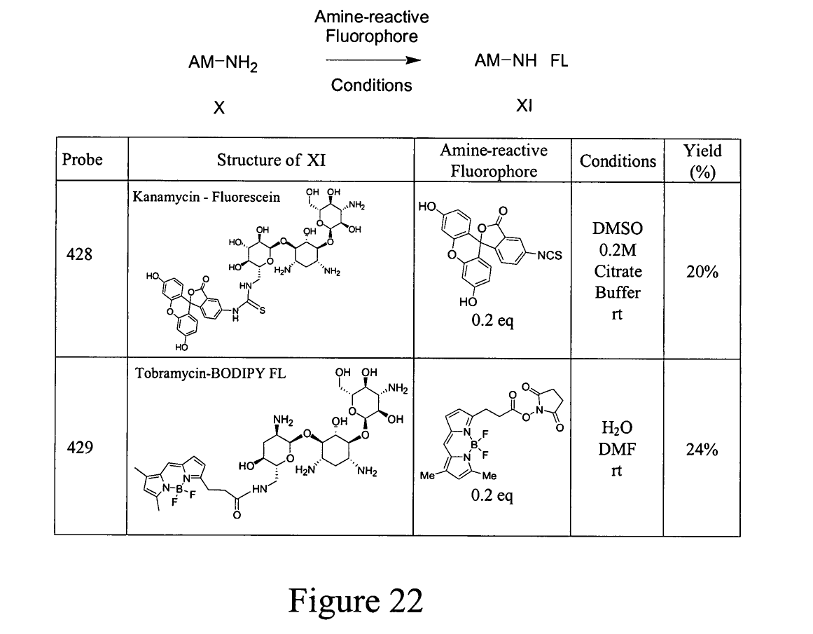

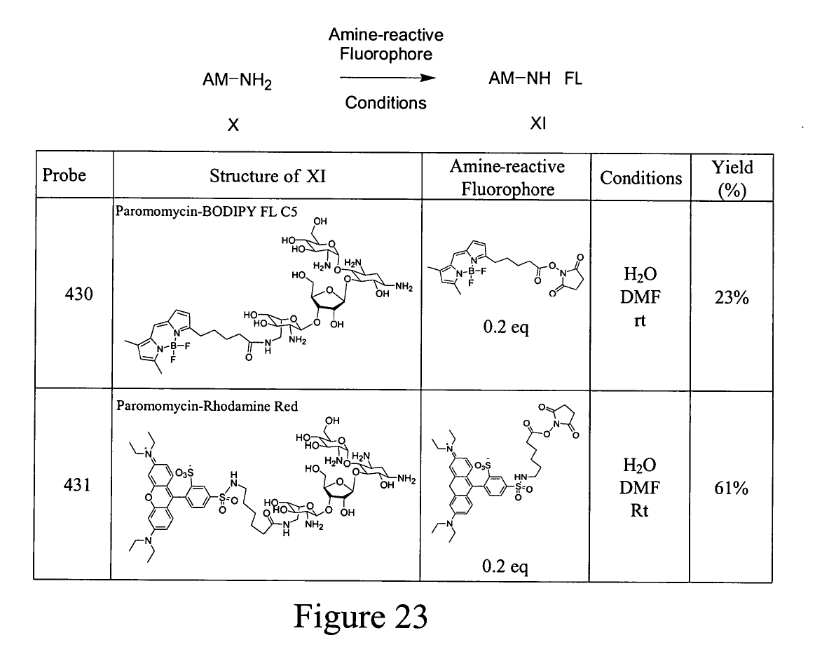

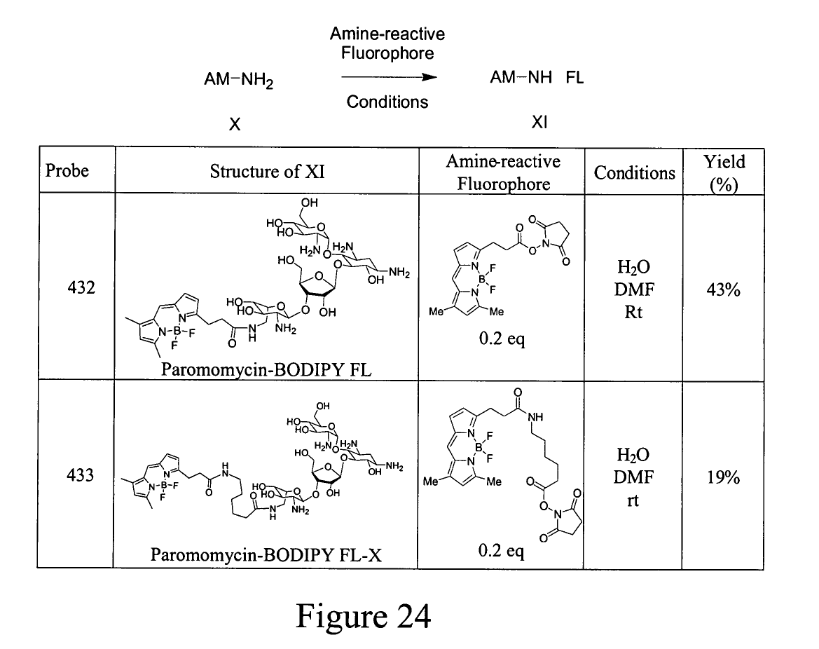

Fluorescent probes based on aminoglycosides are prepared by reacting an aminoglycoside or its salt X with 0.1 to 2.0 equivalents of an amine-reactive fluorophore, in a suitable solvent, in the presence or absence of a base to afford the desired aminoglycoside fluorescent probe XI as illustrated in Scheme E of FIG. 20. The typical solvent suitable for this reaction is DMF, NMP, DMSO, acetone, acetonitrile, THF, ethanol or water. The typical base is sodium carbonate, potassium carbonate, sodium hydroxide, triethylamine, pyridine, DMAP or lutidine. Possible aminoglycosides include but are not limited to kanamycin, gentamycin, tobramycin, amikacin, netilmicin, streptomycin, neomycin, paromomycin, spectinomycin, sisomicin, dibekacin, and isepamicin. The coupling products are purified by HPLC using a C18 reverse phase column. Probes 426-432 of aminoglycoside-based fluorescent probes are shown in FIG. 21, FIG. 22, FIG. 23, and FIG. 24.

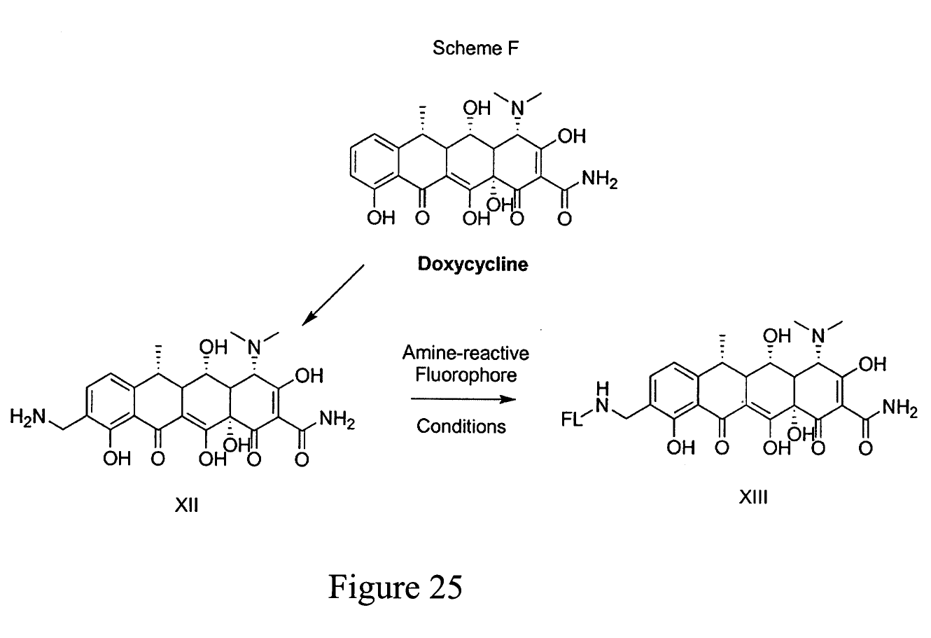

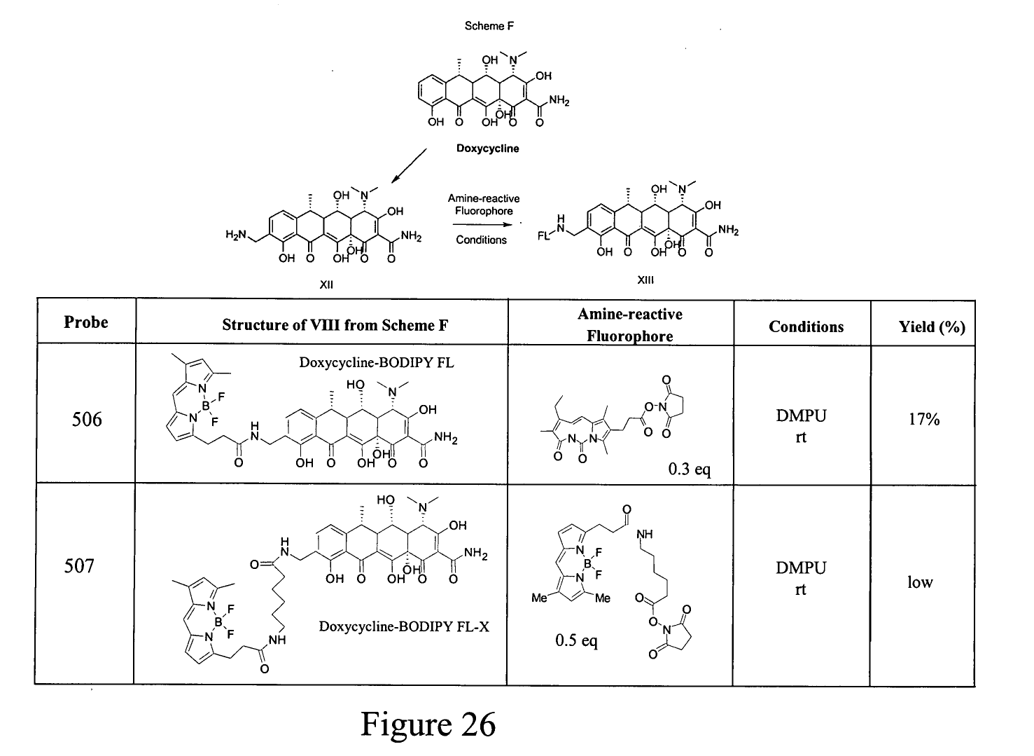

Fluorescent probes based on tetracyclines are prepared according to the synthesis illustrated by Scheme F of FIG. 25. Doxycycline is first converted to 9-aminomethyl doxycycline (XII) according to the literature procedures (Harding, K. E.; Marman, T. H.; Nam, D. Tetrahedron 1988, 44, 5605-5614; Tramontini, M. Synthesis 1973, 703-775). XII reacts with 0.1 to 2.0 equivalents of an amine-reactive fluorophore, in a suitable solvent, in the presence or absence of a base, to afford the desired tetracycline fluorescent probe XIII as illustrated in Scheme F. The typical solvent suitable for this reaction is DMPU, DMF, NMP, DMSO, acetone, acetonitrile, THF, ethanol or water. The typical base is sodium carbonate, potassium carbonate, sodium hydroxide, triethylamine, pyridine, DMAP or lutidine. Other potential tetracyclines include but are not limited to chlortetracycline, demeclocycline, minocycline, oxytetracycline, methacycline and doxycycline. Probes 506 and 507 of tetracycline-based probes are shown in FIG. 26.

The binding of these fluorescent probes to the ribosome and likewise their displacement from the ribosome can be detected using fluorescence polarization or fluorescence intensity technology, resulting in many novel and useful applications. Displacement of the probes enables measurement of the affinity of the ribosome for molecules that show competitive binding. Thus, kits/methods for measuring affinity of ribosome binding molecules are part of this invention. Furthermore, biological samples can be used with related kits/methods to quantify the level of antibiotic or inhibitor in the sample.

Displacement of the probe is useful to screen for molecules that bind to the antibiotic binding site on the ribosome. We have utilized screening conditions and parameters that enabled more sensitive screening than conditions previously reported. The improved detection combined with ribosome sites unexplored under previous art is an important advance for the discovery of novel inhibitors of the ribosome that can serve as antimicrobial agents. The said fluorescent probes also have utility for the discovery of compounds with differential binding to ribosomes of different organisms. The specificity of the fluorescent probes can be studied by comparing the probe's affinity for ribosomes from multiple bacteria, fungi, human cytosol, and human mitochondria. This provides a rapid method for screening selectivity and specificity for the desired target organism with reduced toxicity or side effects to humans. Additionally, probes with sufficient affinity for ribosomes from different organisms can also be used to determine the affinity of a lead compound for ribosomes from different organisms. This again enables the rapid discovery of compounds with improved specificity for the target organism over other organisms and human cells.

Although not wanting to be bound by theory, the fluorescent probes of this invention also have applications for detection of antibiotics within cells. Probes can be used to quantify the level of ribosomes within cells. Fluorescence of the probes can be used to study the penetration and localization of antibiotics into different tissues of animals, into bacterial and fungal biofilms, or into different compartments of bacterial or eukaryotic cells. This enables a better understanding of the pharmacokinetics, toxicity, efficacy, or mechanism of action of that particular class of antibiotics.

Ribosomes from bacterium such as: Acinetobacter calcoaceticus, A. haemolyticus, Aeromonas hydrophilia, Bacteroides fragilis, B. distasonis, Bacteroides 3452A homology group, B. vulgatus, B. ovalus, B. thetaiotaomicron, B. uniformis, B. eggerthii, B. splanchnicus, Branhamella catarrhalis, Campylobacterfetus, C. jejuni, C. coli, Citrobacterfreundii, Clostridium difficile, C. diphtheriae, C. ulcerans, C. accolens, C. afermentans, C. amycolatum, C. argentorense, C. auris, C. bovis, C. confusum, C. coyleae, C. durum, C. falsenii, C. glucuronolyticum, C. imitans, C. jeikeium, C. kutscheri, C. kroppenstedtii, C. lipophilum, C. macginleyi, C. matruchoti, C. mucifaciens, C. pilosum, C. propinquum, C. renale, C. riegelii, C. sanguinis, C. singulare, C. striatum, C. sundsvallense, C. thomssenii, C. urealyticum, C. xerosis, Enterobacter cloacae, E. aerogenes, Enterococcus avium, E. casseliflavus, E. cecorum, E. dispar, E. durans, E. faecalis, E. faecium, E. flavescens, E. gallinarum, E. hirae, E. malodoratus, E. mundtii, E. pseudoavium, E. raffinosus, E. solitarius, Francisella tularensis, Gardnerella vaginalis, Helicobacter pylori, Kingella dentrificans, K. kingae, K. oralis, Klebsiella pneumoniae, K. oxytoca, Moraxella catarrhalis, M. atlantae, M. lacunata, M. nonliquefaciens, M. osloensis, M. phenylpyruvica, Morganella morganii, Parachlamydia acanthamoebae, Pasteurella multocida, P. haemolytica, Proteus mirabilis, Proteus vulgaris, Providencia alcalifaciens, P. rettgeri, P. stuartii, Serratia marcescens, Simkania negevensis, Streptococcus pneumoniae, S. agalactiae, S. pyogenes, Treponema pallidum, Vibrio cholerae, and V. parahaemolyticus are also included as an embodiment of this invention.

Ribosomes from facultative intracellular bacteria such as: Bordetella pertussis, B. parapertussis, B. bronchiseptica, Burkholderia cepacia, Escherichia coli, Haemophilus actinomycetemcomitans, H. aegyptius, H. aphrophilus, H. ducreyi, H. felis, H. haemoglobinophilus, H. haemolyticus, H. influenzae, H. paragallinarum, H. parahaemolyticus, H. parainfluenzae, H. paraphrohaemolyticus, H. paraphrophilus, H. parasuis, H. piscium, H. segnis, H. somnus, H. vaginalis, Legionella adelaidensis, L. anisa, L. beliardensis, L. birminghamensis, L. bozemanii, L. brunensis, L. cherrii, L. cincinnatiensis, Legionella drozanskii L. dumoffli, L. erythra, L. fairfieldensis, L. fallonii, L. feeleii, L. geestiana, L. gormanii, L. gratiana, L. gresilensis, L. hackeliae, L. israelensis, L. jordanis, L. lansingensis, Legionella londiniensis L. longbeachae, Legionella lytica L. maceachernii, L. micdadei, L. moravica, L. nautarum, L. oakridgensis, L. parisiensis, L. pittsburghensis, L. pneumophila, L. quateirensis, L. quinlivanii, L. rowbothamii, L. rubrilucens, L. sainthelensi, L. santicrucis, L. shakespearei, L. spiritensis, L. steigerwaltii, L. taurinensis, L. tucsonensis, L. wadsworthii, L. waltersii, L. worsleiensis, Listeria denitrificans, L. grayi, L. innocua, L. ivanovii, L. monocytogenes, L. seeligeri, L. welshimeri, Mycobacterium abscessus, M. africanum, M. agri, M. aichiense, M. alvei, M. asiaticum, M, aurum, M. austroafricanum, M. avium, M. bohemicum, M. bovis, M. branderi, M. brumae, M. celatum, M. chelonae, M. chitae, M. chlorophenolicum, M. chubuense, M. confluentis, M. conspicuum, M. cookii, M. diernhoferi, M. doricum, M. duvalii, M. elephantis, M. fallax, M. farcinogenes, M. flavescens, M. fortuitum, M. frederiksbergense, M. gadium, M. gastri, M. genavense, M. gilvum, M. goodii, M. gordonae, M. haemophilum, M. hassiacum, M. heckeshornense, M. heidelbergense, M. hiberniae, M. immunogenum, M. intracellulare, M. interjectum, M. intermedium, M. kansasii, M. komossense, M. kubicae, M. lentiflavum, M. leprae, M. lepraemurium, M. luteum, M. madagascariense, M. mageritense, M. malmoense, M. marinum, M. microti, M. moriokaense, M. mucogenicum, M. murale, M. neoaurum, M. nonchromogenicum, M. novocastrense, M. obuense, M. parqfortuitum, M. paratuberculosis, M. peregrinum, M. phage, M. phlei, M. porcinum, M. poriferae, M. pulveris, M. rhodesiae, M. scrofulaceum, M. senegalense, M. septicum, M. shimoidei, M. simiae, M. smegmatis, M. sphagni, M. szulgai, M. terrae, M. thermoresistibile, M. tokaiense, M. triplex, M. triviale, M. tuberculosis, M. tusciae, M. ulcerans, M. vaccae, M. wolinskyi, M. xenopi, Neisseria animalis, N. canis, N. cinerea, N. denitrificans, N. dentiae, N. elongata, N. flava, N. flavescens, N. gonorrhoeae, N. iguanae, N. lactamica, N. macacae, N. meningitidis, N. mucosa, N. ovis, N. perflava, N. pharyngis var. flava, N. polysaccharea, N. sicca, N. subflava, N. weaveri, Pseudomonas aeruginosa, P. alcaligenes, P. chlororaphis, P. fluorescens, P. luteola, P. mendocina, P. monteilii, P. oryzihabitans, P. pertocinogena, P. pseudalcaligenes, P. putida, P. stutzeri, Salmonella bacteriophage, S. bongori, S. choleraesuis, S. enterica, S. enteritidis, S. paratyphi, S. typhi, S. typhimurium, S. typhimurium, S. typhimurium, S. typhimurium bacteriophage, Shigella boydii, S. dysenteriae, S. flexneri, S. sonnei, Staphylococcus arlettae, S. aureus, S. auricularis, S. bacteriophage, S. capitis, S. caprae, S. carnosus, S. caseolyticus, S. chromogenes, S. cohnii, S. delphini, S. epidermidis, S. equorum, S. felis, S. fleurettii, S. gallinarum, S. haemolyticus, S. hominis, S. hyicus, S. intermedius, S. kloosii, S. lentus, S. lugdunensis, S. lutrae, S. muscae, S. mutans, S. pasteuri, S. phage, S. piscifermentans, S. pulvereri, S. saccharolyticus, S. saprophyticus, S. schleiferi, S. sciuri, S. simulans, S. succinus, S. vitulinus, S. warneri, S. xylosus, Ureaplasma urealyticum, Yersinia aldovae, Y. bercovieri, Y. enterocolitica, Y. frederiksenii, Y. intermedia, Y. kristensenii, Y. mollaretii, Y. pestis, Y. philomiragia, Y. pseudotuberculosis, Y. rohdei, and Y. ruckeri are also included as an embodiment of this invention.

Ribosomes from obligate intracellular bacteria, such as: Anaplasma bovis, A. caudatum, A. centrale, A. marginale A. ovis, A. phagocytophila, A. platys, Bartonella bacilliform is, B. clarridgeiae, B. elizabethae, B. henselae, B. henselae phage, B. quintana, B. taylorii, B. vinsonii, Borrelia afzelii, B. andersonii, B. anserina, B. bissettii, B. burgdorferi, B. crocidurae, B. garinii, B. hermsii, B. japonica, B. miyamotoi, B. parkeri, B. recurrentis, B. turdi, B. turicatae, B. valaisiana, Brucella abortus, B. melitensis, Chlamydia pneumoniae, C. psittaci, C. trachomatis, Cowdria ruminantium, Coxiella burnetii, Ehrlichia canis, E. chaffeensis, E. equi, E. ewingii, E. muris, E. phagocytophila, E. platys, E. risticii, E. ruminantium, E. sennetsu, Haemobartonella canis, H. felis, H. muris, Mycoplasma arthriditis, M. buccale, M. faucium, M. fermentans, M. genitalium, M. hominis, M. laidlawii, M. lipophilum, M. orale, M. penetrans, M. pirum, M. pneumoniae, M. salivarium, M. spermatophilum, Rickettsia australis, R. conorii, R. felis, R. helvetica, R. japonica, R. massiliae, R. montanensis, R. peacockii, R. prowazekii, R. rhipicephali, R. rickettsii, R. sibirica, and R. typhi are also included as an embodiment of this invention.

Ribosomes from facultative intracellular fungi, such as: Candida Candida aaseri, C. acidothermophilum, C. acutus, C. albicans, C. anatomiae, C. apis, C. apis var. galacta, C. atlantica, C. atmospherica, C. auringiensis, C. bertae, C. berthtae var. chiloensis, C. berthetii, C. blankii, C. boidinii, C. boleticola, C. bombi, C. bombicola, C. buinensis, C. butyri, C. cacaoi, C. cantarellii, C. cariosilignicola, C. castellii, C. castrensis, C. catenulata, C. chilensis, C. chiropterorum, C. coipomensis, C. dendronema, C. deserticola, C. diddensiae, C. diversa, C. entomaea, C. entomophila, C. ergatensis, C. ernobii, C. ethanolica, C. ethanothermophilum, C. famata, C. fluviotilis, C. fragariorum, C. fragicola, C. friedrichii, C. fructus, C. geochares, C. glabrata, C. glaebosa, C. gropengiesseri, C. guilliermondii, C. guilliermondii var. galactosa, C. guilliermondii var. soya, C. haemulonii, C. halophila/C. versatilis, C. holmii, C. humilis, C. hydrocarbofumarica, C. inconspicua, C. insectalens, C. insectamans, C. intermedia, C. javanica, C. kefyr, C. krissii, C. krusei, C. krusoides, C. lambica, C. lusitaniae, C. magnoliae, C. maltosa, C. mamillae, C. maris, C. maritima, C. melibiosica, C. melinii, C. methylica, C. milleri, C. mogii, C. molischiana, C. montana, C. multis-gemmis, C. musae, C. naeodendra, C. nemodendra, C. nitratophila, C. norvegensis, C. norvegica, C. oleophila, C. oregonensis, C. osornensis, C. paludigena, C. parapsilosis, C. pararugosa, C. periphelosum, C. petrohuensis, C. petrophilum, C. philyla, C. pignaliae, C. pintolopesii var. pintolopesii, C. pintolopesii var. slooffiae, C. pinus, C. polymorpha, C. populi, C. pseudointermedia, C quercitrasa, C. railenensis, C. rhagii, C. rugopelliculosa, C. rugosa, C. sake, C. salmanticensis, C. savonica, C. sequanensis, C. shehatae, C. silvae, C. silvicultrix, C. solani, C. sonorensis, C. sorbophila, C. spandovensis, C. sphaerica, C. stellata, C. succiphila, C. tenuis, C. terebra, C. tropicalis, C. utilis, C. valida, C. vanderwaltii, C. vartiovaarai, C. veronae, C. vini, C. wickerhamii, C. xestobii, C. zeylanoides, and Histoplasma capsulatum are also included as an embodiment of this inention.

Ribosomes from obligate intracellular protozoans, such as: Brachiola vesicularum, B. connori, Encephalitozoon cuniculi, E. hellem, E. intestinalis, Enterocytozoon bieneusi, Leishmania aethiopica, L. amazonensis, L. braziliensis, L. chagasi, L. donovani, L. donovani chagasi, L. donovani donovani, L. donovani infantum, L. enriettii, L. guyanensis, L. infantum, L. major, L. mexicana, L. panamensis, L. peruviana, L. pifanoi, L. tarentolae, L. tropica, Microsporidium ceylonensis, M. africanum, Nosema connori, N. ocularum, N. algerae, Plasmodium berghei, P. brasilianum, P. chabaudi, P. chabaudi adami, P. chabaudi chabaudi, P. cynomolgi, P. falciparum, P. fragile, P. gallinaceum, P. knowlesi, P. lophurae, P. malariae, P. ovale, P. reichenowi, P. simiovale, P. simium, P. vinckeipetteri, P. vinckei vinckei, P. vivax, P. yoelii, P. yoelii nigeriensis, P. yoelii yoelii, Pleistophora anguillarum, P. hippoglossoideos, P. mirandellae, P. ovariae, P. typicalis, Septata intestinalis, Toxoplasma gondii, Trachipleistophora hominis, T. anthropophthera, Vittaforma corneae, Trypanosoma avium, T. brucei, T. brucei brucei, T. brucei gambiense, T. brucei rhodesiense, T. cobitis, T. congolense, T. cruzi, T. cyclops, T. equiperdum, T. evansi, T. dionisii, T. godfreyi, T. grayi, T. lewisi, T. mega, T. microti, T. pestanai, T. rangeli, T. rotatorium, T. simiae, T. theileri, T. varani, T. vespertilionis, and T. vivax are also included as an embodiment of this invention.

A fluorescence binding assay utilizing the probes can be used in parallel with a biochemical assay (e.g. transcription and translation assay) to demonstrate that inhibition is directly linked to the ribosome binding. The probes can be used to screen for compounds that cause an increased fluorescence polarization or a quenching of fluorescence intensity because they bind synergistically with probe. The probes can be used as tools for detecting specific ribosome states to allow targeting of specific ribosome states and/or locking of ribosomes in specific conformations.

### EXAMPLES

The invention may be better understood with reference to the following examples, which are representative of some of the embodiments of the invention, and are not intended to limit the invention.

### Example I

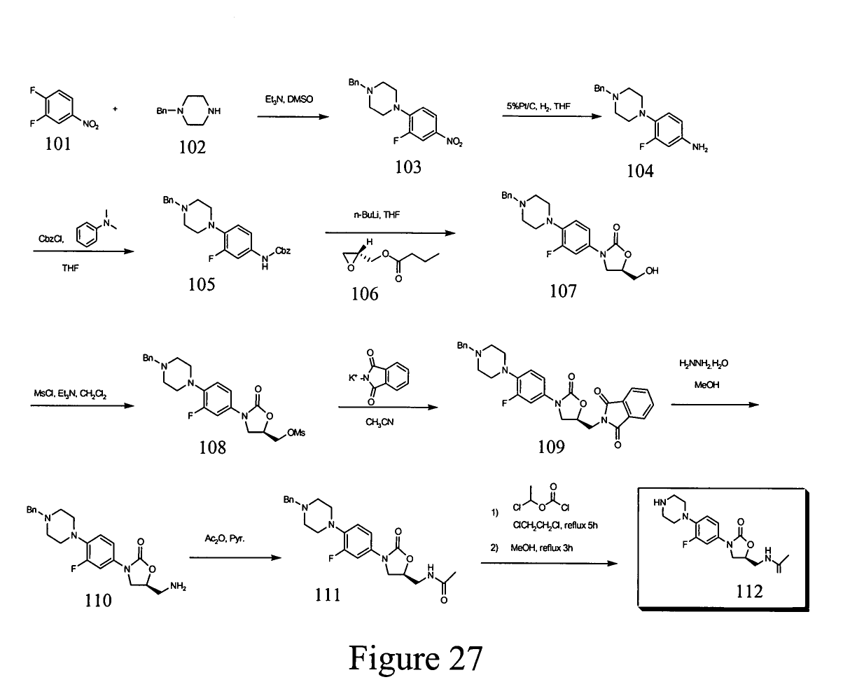

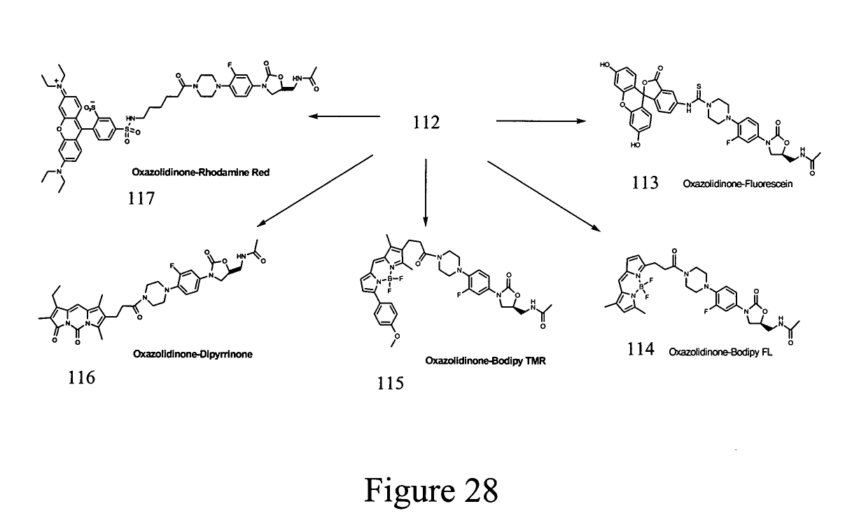

Oxazolidinone Probes. One series of probes of this invention are based on oxazolidinones. FIG. 27 illustrates the synthesis to prepare the oxazolidinone core compound 112. FIG. 28 illustrates the synthesis comprising compound 112 being reacted with different activated fluorophors to give a variety of oxazolidinone probes under typical coupling conditions. For example, FIG. 27 shows that (1-benzyl-4-(2-fluoro-4-nitro-phenyl)-piperazine) (“103”) was obtained as follows: Step 1, to a solution of difluoronitrobenzene (“101”) (1.08 mL, 9.8 mmol) and benzylpiperazine (“102”) (1.8 mL, 10.4 mmol) in CH₃CN (10 mL) was added triethylamine (1.4 mL, 10.0 mmol). The resulting solution was heated at 90° C. for 3.5 h and then diluted with EtOAc and H₂O. The organic phase was separated and washed with H₂O, brine and dried over Na₂SO₄. The solvent was evaporated in vacuum to afford a yellow solid 103 (3.68g). Compound 103: TLC (20% EtOAc/ Hexane) R_{f}=0.40. ¹H NMR (400 MHz, CDCl₃): δ (ppm) 2.64 (app t, J=4.8 Hz, 4H), 3.32 (app t, J=4.8 Hz, 4H), 3.59 (s, 2H), 6.90 (t, J=8.8 Hz, 1H), 7.27-7.36 (m, 5H), 7.90 (dd, J=2.4, 13.2 Hz, 1H), 7.98 (dd, J=2.4, 8.8 Hz, 1H).

Step 2: The (4-(4-benzyl-piperazin-1-yl)-3-fluoro-pheny)-carbamic acid benzyl ester (“105”) in FIG. 27 was obtained as follows: To a solution of 103 (17.4 g, 55.2 mmol) in THF (350 mL) was added 5% Pt—C (2.1 g), and stirred under H₂ atmosphere (1 atm) for 16 h. The catalyst was filtered and condensation of the solvent afforded the yellow solid 104 (16 g). To the solution of 104 (-16 g, 55.2 mmol) and dimethylamine (7.2 mL, 56.8mmol) in THF (300 mL) was added dropwise at 0° C. benzyl chloroformate (8.0 mL, 56.0 mmol). The resulting solution was stirred at 0° C. for 15 min and then at r.t. for 30 min. About two/thirds of the solvent was removed and the residue was diluted with CH₂Cl₂ (500 mL). The solution was washed subsequently with 1N HCl, H₂O, brine, and dried over Na₂SO₄. The solution was condensed and purified by chromatography with 20-30% EtOAc/Hexane to afford yellow wax 105 (22.0g, 95% over two steps). Compound 105: TLC (50% EtOAc/Hexane) R_{f}=0.32. ¹H NMR (400MHz, CDCl₃): δ (ppm) 3.02 (app q, J=12.0 Hz, 2H), 3.32 (app d, J=12.0 Hz, 2H),3.45 (app d, J=12.0 Hz, 2H), 3.63 (app t, J=12.0 Hz, 2H), 4.20 (d, J=4.8 Hz, 2H), 5.19(s, 2H), 6.84-6.93 (m, 3H), 7.34-7.47 (m, 8H), 7.66-7.68 (m, 2H).

Step 3: The 3-(4-(4-benzyl-piperazin-1-yl)-3-fluoro-phenyl)-5-hydroxymethyl-oxazolidin-2-one (“107”) in FIG. 27 was obtained as follows: A solution of 105 (6.0 g, 14.3 mmol) in anhydrous THF (240 mL) was cooled to −78° C. and added n-BuLi (1.6 M. solution in hexane, 10.0 mL) dropwise. The resulting solution was stirred at −78° C. for 30 min and added (R)-(−)-glycidyl butyrate (“106”) (2.0 mL, 14.3 mmol). The reactant was warmed up to r.t. for 2 h and then stirred at 30° C. for 2 h, and subsequently diluted with EtOAc and H₂O. The organic phase was separated and washed with H₂O, brine and dried over Na₂SO₄. Condensation and chromatography with 75% to 100% EtOAc in Hexane afforded a white solid 107 (3.6 g, 65% yield). Compound 107: TLC (EtOAc) R_{f}=0.10. ¹H NMR (400 MHz, CDCl₃): δ (ppm) 2.14 (br s, 1H), 2.64 (app s, 4H), 3.08 (app s, 4H), 3.59 (s, 2H), 3.76 (dd, J=4.0, 12.8 Hz, 1H), 3.92-4.02 (m, 3H), 4.82-4.76 (m, 1H), 6.94 (t, J=8.0 Hz, 1H), 7.11 (dd, J=2.4, 8.8 Hz, 1H),7.27-7.39 (m, 5H), 7.43 (dd, J=2.4, 14.4 Hz, 1H).

Step 4. The 2-(3-(4-benzyl-piperazin-1-yl)-3-fluoro-phenyl)-2-oxo-oxazolidin-5-ylmethyl)-isoindole-1,3-dione (“109”) in FIG. 27 was obtained as follows: A solution of 107 (3.9 g, 10.2 mmol) in dichloromethane (100 mL) was cooled to 0° C. and triethylamine (2.8 mL, 20.1 mmol) and methanesulfonyl chloride (1.0 mL, 13.2 mmol) were added. The resulting solution was stirred at 0° C. for 1 h, and then washed with water, sat. Na₂CO₃, water and brine. The organic layer was dried over sodium sulfate and condensed to afford an off-white solid 108 (4.6 g, 10.0 mmol), which was dissolved in acetonitrile (250 mL) with potassium phthalimide (5.6 g, 30.3 mmol) and heated at 95° C. for 40 h. The precipitation was filtered off and the filtration was condensed to 30 mL. The crystalline from the condensed solution was collected by filtration and washed with CH₃CN /Et₂O (5 mL×3) to yield a white solid 109 (3.2 g, 62% yield overall two steps). Compound 108: TLC (EtOAc) R_{f}=0.36.¹H NMR (400 MHz, CDCl₃): δ (ppm) 2.64 (app s, 4H), 3.09 (app s, 4H), 3.11 (s, 3H), 3.59 (s, 2H), 3.91 (dd, J=6.4, 9.2 Hz, 1H), 4.11 (t, J=9.2 Hz, 1H), 4.42 (dd, J_{AB}=4.0, 11.6 Hz, 1H), 4.50 (dd, J_{AB}=4.0, 11.6 Hz, 1H), 4.89-4.93 (m, 1H), 6.95 (t, J=8.8 Hz, 1H), 7.09 (d, J=8.8 Hz, 1H),7.27-7.36 (m, 5H), 7.43 (dd, J=2.0, 14.0 Hz, 1H). ES-MS (m/z): 464.1 (M+H)⁺. Compound 109: TLC (50% EtOAc/Hexane) R_{f}=0.35.¹H NMR (400 MHz, CDCl₃): δ (ppm) 2.61 (app s, 4H), 3.04 (app s, 4H), 3.55 (s, 2H), 3.83 (dd, J=5.6, 8.8 Hz, 1H), 3.94 (dd, J_{AB}=6.4, 13.6 Hz, 1H),4.03 (t, J=8.4 Hz, 1H), 4.11 (dd, J_{AB}=6.4, 13.6 Hz, 1H), 4.91-4.96 (m, 1H), 6.89 (t, J=9.2 Hz, 1H), 7.06 (dd, J=2.4, 8.4 Hz, 1H),7.23-7.38 (m, 6H), 7.73-7.75 (m, 2H), 7.85-7.87 (m, 2H).

Step 5. The N-3-(4-(4-benzyl-piperazin-1-yl)-3-fluoro-phenyl)-2-oxo-oxazolidin-5-ylmethyl) acetamide (“111”) in FIG. 27 was obtained as follows: To a suspension of 109 (1.0 g, 2.0 mmol) in methanol (20 mL) was added hydrazine (0.1 mL, 4.1 mmol) and the mixture was heated to reflux for 6 h. The reactant was poured into 60 mL 3% K₂CO₃ and extracted with EtOAc (40 mL×2). The combined organic phase was washed with brine, dried over MgSO₄ and condensed to afford a white solid 110 (0.76 g). To the solution of the above product in 10 mL pyridine was added acetic anhydride (3.4 mL) and stirred at r.t. overnight. The reactant was diluted with EtOAc and H₂O. The organic phase was separated and washed with H₂O, brine and dried over Na₂SO₄. Condensation and chromatography with 10% MeOH/CH₂Cl₂ afforded a white solid 111 (370 mg, 44% yield). Compound 110: TLC (75%EtOAc/Hexane) R_{f}=0.56.¹H NMR (400 MHz, CDCl₃): δ (ppm) 2.61 (app s, 4H), 2.95 (dd, J_{AB}=6.0, 13.6 Hz, 1H), 3.05 (app s, 4H), 3.09 (app dd, J_{AB}=6.0, 13.6 Hz, 1H), 3.55 (s, 2H), 3.78 (t, J=7.4 Hz, 1H), 3.98 (t, J=8.6 Hz, 1H), 4.61-4.65 (m, 1H), 6.91 (t, J=9.2 Hz, 1H), 7.10 (dd, J=2.4, 8.8 Hz, 1H), 7.27-7.38 (m, 5H), 7.42 (dd, J=2.4, 14.4 Hz, 1H). Compound 111: TLC (10% MeOH/CH₂Cl₂) R_{f}=0.70. ¹H NMR (400 MHz, CDCl₃): δ (ppm) 1.99 (s, 3H), 2.62 (app s, 4H), 3.06 (app s, 4H), 3.56 (s, 2H), 3.55-3.62 (m, 1H), 3.66 (dd, J=2.8, 6.0 Hz, 1H), 3.70 (dd, J=7.2, 9.2 Hz, 1H), 3.99 (t, J=9.2 Hz, 1H), 4.72-75 (m, 1H), 6.03 (br s, 1H), 6.89 (t, J=9.2 Hz, 1H), 7.03 (dd, J=2.0, 8.8 Hz, 1H), 7.24-7.37 (m, 5H), 7.39 (dd, J=2.4, 14.8 Hz, 1H).

Step 6. The N-(3-(3-fluoro-4-piperazin-1-yl-phenyl)-2-oxo-oxazolidin-5-ylmethyl)acetamide (“112”) in FIG. 27 was obtained as follows: To a solution of 111 (20 mg, 0.05 mmol) in dichloroethane (0.3 mL) was added 1-chloroethyl chloroformate (5.8 μL, 0.05 mmol) and heated at 85° C. in sealed tube for 4 h. After removing solvent, the residue was dissolved in MeOH (1.5 mL) and heated to reflux for 3 h. PTLC with 10% MeOH/CH₂Cl₂ afforded a white solid 112 (9.6 mg, 57% Yield). Compound 112: TLC (10% MeOH/CH₂Cl₂) R_{f}=0.08. ¹H NMR (400 MHz, CD₃OD): δ (ppm) 1.96 (s, 3H), 3.14 (app s, 8H), 3.56 (d, J=4.8 Hz, 2H), 3.79 (dd, J=6.4, 9.2 Hz, 1H), 4.12 (t, J=9.2 Hz, 1H), 4.76-80 (6 lines m, 1H), 7.08 (t, J=9.2 Hz, 1H), 7.18 (dd, J=1.6, 9.2 Hz, 1H), 7.51 (dd, J=2.8, 14.4 Hz, 1H).

Step 7. As shown in FIG. 28 and described below, the probe N-3-(4-(4-fluorescein-piperazin-1 -yl)-3 -fluoro-phenyl)-2-oxo-oxazolidin-5 -ylmethyl)acetamide (“113”) was obtained as follows: To a solution of 112 (7.0 mg, 0.020 mmol) in acetone/H₂O (0.2 mL/0.2 mL) was added K₂CO₃ (8.4 mg, 0.060 mmol) and fluorescein isothiocyanate (9.8 mg, 0.025 mmol). The resulting solution was stirred at r.t. overnight, and the solvent was removed under vacuum. The residue was purified by chromatography with 5-15% MeOH/CH₂Cl₂ which afforded yellow solid 113 (11.6 mg, 80% yield). Compound 113: TLC (15% MeOH/CH₂Cl₂) R_{f}=0.60. ¹H NMR (400 MHz, CD₃OD): δ (ppm) 1.97 (s, 3H), 3.17 (app s, 4H), 3.56 (d, J=4.8 Hz, 2H), 3.81 (dd, J=6.0, 9.2 Hz, 1H), 4.13 (t, J=9.2 Hz, 1H), 4.19 (app s, 4H), 4.76-4.80 (m, 1H), 6.57 (dd, J=2.4, 8.8 Hz, 2H), 6.68 (d, J=2.4 Hz, 2H), 6.79 (d, J=8.8 Hz, 2H), 7.09-7.21 (m, 3H), 7.54 (dd, J=2.4, 10.6 Hz, 1H), 7.73 (dd, J=2.0, 8.4 Hz, 1H), 7.98 (s, 1H). ES-MS (m/z): 726.1 (M+H)⁺

As shown in FIG. 28 and described below, the probe N-3-(4-(4-Bodipy FL-piperazin-1-yl)-3-fluoro-phenyl)-2-oxo-oxazolidin-5-ylmethyl)acetamide (“114”) was obtained as follows: To a solution of 112 (2.8 mg, 0.008 mmol) in DMF (0.12 mL) was added Bodipy FL SE (Molecular Probes, 3.8 mg, 0.010 mmol) and stirred at r.t. overnight. After removal of solvent under vacuum, the residue was purified by chromatography with 5% MeOH/CH₂Cl₂ to afford an orange solid 114 (4.8 mg, 95% yield). Compound 114: TLC (5% MeOH/CH₂Cl₂) R_{f}=0.30. ¹H NMR (400 MHz, CD₃OD): δ (ppm) 1.96 (s, 3H), 2.29 (s, 3H), 2.52 (s, 3H), 2.86 (t, J=7.6 Hz, 2H), 2.96 (app t, J=5.2 Hz, 2H), 3.00 (app t, J=5.2 Hz, 2H), 3.24 (t, J=7.6Hz, 2H), 3.55 (d, J=4.4 Hz, 2H), 3.69 (app t, J=4.8 Hz, 2H), 3.76 (app t, J=4.8 Hz, 2H), 3.78 (dd, J=6.4, 9.2 Hz, 1H), 4.11 (t, J=9.2 Hz, 1H), 4.75-4.80 (6 lines m, 1H), 6.23 (s, 1H), 6.36 (d, J=4.4 Hz, 1H), 7.00 (d, J_{AB}=7.8 Hz, 1H), 7.03 (d, J_{AB}=7.8 Hz, 1H), 7.45 (s, 1H), 7.49 (dd, J=2.8, 14.8 Hz, 1H). ES-MS (m/z): 611.0 (M+H)⁺.

As shown in FIG. 28 and described below, the probe N-3-(4-(4-Bodipy TMR-piperazin-1 -yl)-3-fluoro-phenyl)-2-oxo-oxazolidin-5-ylmethyl)acetamide (“115”) was obtained as follows: To a solution of 112 (3.2 mg, 0.010 mmol) in DMF (0.10 mL) was added Bodipy TMR STP ester (Molecular Probes, 1.2 mg, 0.002 mmol) and stirred at r.t. overnight. After removal of solvent under vacuum, the residue was purified by PTLC with 10% MeOH/CH₂Cl₂ to afford an orange solid 115 (1.1 mg). Compound 115: TLC (5% MeOH/CH₂Cl₂) R_{f}=0.30. ¹H NMR (400 MHz, CD₃OD): δ (ppm) 1.96 (s, 3H), 2.27 (s, 3H), 2.46-2.50 (m, 2H), 2.54 (s, 3H), 2.64 (t, J=6.8 Hz, 2H), 2.79-2.83 (m, 2H), 2.87 ( t, J=6.8 Hz, 2H), 3.51 (d, J=5.2 Hz, 2H), 3.55 (app t, J=7.6 Hz, 2H), 3.66 (dd, J=6.4, 9.2 Hz, 1H), 3.68-3.72 (m, 2H), 3.99 (t, J=9.0 Hz, 1H), 4.70-4.76 (6 lines m, 1H), 6.65-6.71 (4 lines m, 2H), 6.78 (dd, J=2.4, 8.8 Hz, 1H), 6.98 (app d, J=8.8 Hz, 2H), 7.09 (d, J=4.0 Hz, 1H), 7.39 (dd, J=2.8, 14.4 Hz, 1H), 7.45 (s, 1H), 7.89 (app d, J=8.8 Hz, 2H). ES-MS (m/z): 717.4 (M+H)⁺.

As shown in FIG. 28 and described below, the probe N-3-(4-(4-dipyrrinone-piperazin-1 -yl)-3-fluoro-phenyl)-2-oxo-oxazolidin-5-ylmethyl)acetamide (“116”) was obtained as follows: To a solution of 112 (6.0 mg, 0.018 mmol) in DMF (0.20 mL) was added dipyrrinone (Justin O. Brower; David A. Lightner J. Org. Chem. 2002, 67, 2713-1716) (3.0 mg, 0.007 mmol) and stirred at r.t. overnight. After removal of solvent under vacuum, the residue was purified by PTLC with 10% MeOH/CH₂Cl₂ to afford a yellow solid 116 (1.0 mg). Compound 116: TLC (10% MeOH/CH₂Cl₂) R_{f}=0.08. ¹H NMR (400 MHz, CD₃OD): δ (ppm) 1.19 (t, J=7.6 Hz, 3H), 1.92 (s, 3H), 1.97 (s, 3H), 2.20 (s, 3H), 2.44-2.52 (m, 2H), 2.58-2.63 (m, 4H), 2.64 (s, 3H), 2.83 -2.86 (m, 4H), 3.53-3.57 (m, 4H), 3.70-3.78 (m, 3H), 4.09 (t, J=9.2 Hz, 1H), 4.76-4.81 (6 lines m, 1H), 6.78 (t, J=9.2 Hz, 1H), 6.84 (s, 1H), 7.05 (dd, J=1.6, 9.2 Hz, 1H), 7.41 (dd, J=2.4, 14.4 Hz, 1H). ES-MS (m/z): 647.33 (M+H)⁺.

As shown in FIG. 28 and described below, the probe N-3-(4-(4-Rhodamine Red-piperazin-1-yl)-3-fluoro-phenyl)-2-oxo-oxazolidin-5-ylmethyl)acetamide (“117”) was obtained as follows: To a solution of 112 (4.0 mg, 0.012 mmol) in DMF (0.12 mL) was added Rhodamine Red SE (0.7 mg, 0.001 mmol) and stirred at r.t. overnight. After removal of solvent under vacuum, the residue was purified by PTLC with 10% MeOH/CH₂Cl₂ to afford a red solid 117 (0.9 mg). Compound 117: TLC (10% MeOH/CH₂Cl₂) R_{f}=0.60. ¹H NMR (400 MHz, CD₃OD): δ (ppm) 1.29 (t, J=7.0 Hz, 12H), 1.37-1.42 (m, 2H), 1.50-1.55 (m, 2H), 1.59-1.64 (m, 2H), 1.95 (s, 3H), 2.44 (t, J=7.2 Hz, 2H), 2.95-3.00 (m, 4H), 3.06 (t, J=7.2 Hz, 2H), 3.54 (d, J=4.8 Hz, 2H), 3.65-3.73 (m, 12H), 3.77 (dd, J=6.4, 9.6 Hz, 2H), 3.68-3.72 (m, 2H), 4.09 (t, J=9.0 Hz, 1H), 4.74-4.79 (6 lines m, 1H), 6.92 (d, J=2.0 Hz, 2H), 7.00 (d, J_{AB}=9.2 Hz, 2H), 7.02 (t, J=8.4 Hz, 1H), 7.10 (d, J_{AB}=9.2 Hz, 2H), 7.14 (dd, J=2.4, 8.8 Hz, 1H), 7.47(d, J_{AB}=2.8 Hz, 1H), 7.50(d, J_{AB}=2.8 Hz, 1H), 8.10 (dd, J=2.4, 8.4 Hz, 1H), 8.65 (d, J=2.0 Hz, 1H). ES-MS (m/z): 988.4 (M+H)⁺.

### Example II

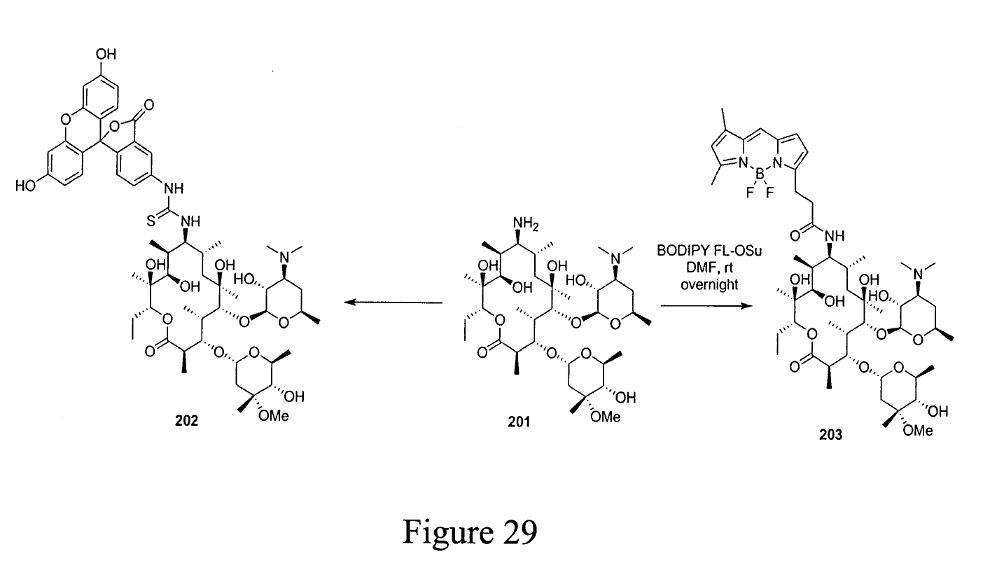

Macrolide Probes. Another series of probes of this invention are based on Macrolides. FIG. 29 illustrates the preparation of 9N-fluorescein erythromycylamine (“202”). To a stirred solution of erythromycylamine (Timms, G. H. et al. Tetrahedron Lett., 1971, 195-198. 0.10 mmol) and K₂CO₃ (28 mg, 0.20 mmol) in acetone-water (2 ml) was added 5-fluorescein isothiocyanate (39 mg, 0.10 mmol). The reaction mixture was stirred at r.t. for 20 hrs and the solvent was evaporated. The residue was purified by column chromatography (silica gel, 1% HOAc in ethyl acetate then methanol) to give an orange solid (28 mg, 25%): MS(M+H)⁺1124.

FIG. 29 illustrates the synthesis necessary to prepare the 9-BODIPY-amino-erythromycin{9-(4,4-difluoro-5,7-dimethyl-4-bora-3a,4a-diaza-s-indacene-3-propionyl)-amino-erythromycin} (“203”) as follows: To a solution of 9-amino-erythromycin (“201”) in DMF (0.5 mL) was added BODIPY FL SE (4,4-difluoro-5,7-dimethyl-4-bora-3a,4a-diaza-s-indacene-3-propionic acid succinimidyl ester) (1 mg) and the resulting mixture was stirred r.t. overnight. After DMF was removed under vacuum, the residue was purified by PTLC (CH₂Cl₂:MeOH:NH₄OH=80:20:1) to give 2 mg of the desired probe 203 in 77% yield based on the used amount of BODIPY FL SE. Compound 203: MS (M+H)⁺1009; ¹H NMR (400 MHz, CD₃OD) δ 7.36 (s, 1H), 6.92 (d, J=4.0 Hz, 1H), 6.28 (d, J=4.4 Hz, 1H), 6.16 (s, 1H), 5.00 (br s, 1H), 4.89 (dd, J=10.0 Hz and 2.4 Hz, 1 H), 4.05-4.01 (m, 1H), 3.88 (br s, 1H), 3.72 (br s, 1H), 3.53 (dd, J=10.0 Hz and 3.6 Hz, 1H), 3.47 (br s, 1H), 3.31 (s, 3H), 3.17 (t, J=7.6 Hz, 2H), 2.99 (d, J=9.6 Hz, 1H), 2.84-2.76 (m, 2H), 2.58 (t, J=8.0 Hz, 2H), 2.46 (s, 3H), 2.42 (s, 1H), 2.35 (s, 3H), 2.23 (s, 3H), 2.12 (m, 1H), 1.86-1.74 (m, 3H), 1.59-1.54 (m, 2H), 1.46-1.38 (m, 1H), 1.24-1.06 (m, 24 H), 0.94 (d, J=6.8 Hz, 3H), 0.82 (t, J=7.6 Hz, 3H).

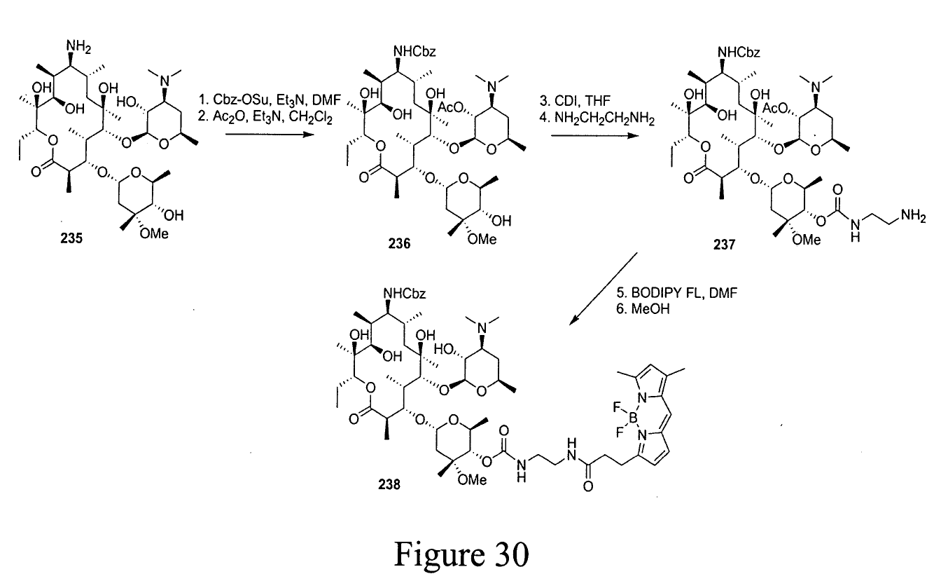

FIG. 30 illustrates the synthesis necessary to prepare probe 238. Step 1, as shown in FIG. 30 and described below, 9-benzyloxycarbonylamino-2′-acetoxy erythromycin (“236”) was synthesized as follows: To a solution of 9-aminoerythromycin (“235”) (44 mg, 0.06 mmol) in DMF (0.7 mL) was added N-(benzyloxycarbonyloxy) succinimide (18 mg, 0.07 mmol) and the resulting mixture was stirred at r.t. overnight. The reaction solution was diluted with EtOAc/H₂O, the separated organic layer was washed with brine, dried over Na₂SO₄ and condensed. The crude material was purified by chromatography with 10% MeOH/CH₂Cl₂ (containing 0.5% ammonium) and afforded 40 mg of product. To the solution of this product (40 mg, 0.05 mmol) in CH₂Cl₂ (0.7 mL) was added triethylamine (30 μL, 0.22 mmol) and acetic anhydride (5.5 μL, 0.05 mmol) and stirred at r.t. for two days. The reaction solution was diluted with EtOAc/H₂O, the separated organic layer was washed with brine and dried over Na₂SO₄. Condensation afforded 40 mg of white solid 236 (73% yield overall two steps). Compound 236: TLC (10% MeOH/CH₂Cl₂) R_{f}=0.45. ¹H NMR (400 MHz, CD₃OD): δ (ppm) 0.90 (t, J=7.2 Hz, 3H), 0.98 (d, J=7.2 Hz, 3H), 1.07 (d, J=7.2 Hz, 3H), 1.08 (s, 3H), 1.17-1.32 (m, 22H), 1.35 (d, J=11.2 Hz, 1H), 1.40-1.46 (m,1H), 1.50-1.56 (m, 1H), 1.63 (dd, J=4.0, 11.2 Hz, 1H), 1.64-1.70 (m, 1H), 1.73 (dd, J=4.0, 11.2 Hz, 1H), 1.89-1.94 (m, 1H), 2.04 (s, 3H), 2.14-2.18 (m, 1H), 2.28 (s, 6H), 2.36-2.40 (m, 2H), 2.68 (dt, J=3.2, 11.2 Hz,1H), 2.80-2.84 (m, 1H), 3.08 (t, J=9.2 Hz, 1H), 3.26 (br s, 1H), 3.30-3.33 (m, 2H)m 3.38 (s, 3H), 2.56-3.62 (m, 1H), 3.73-3.76 (m, 2H), 3.93-3.97 (m, 1H), 4.12-4.16 (m, 1H), 4.61 (d, J=9.2 Hz, 1H), 4.73 (app d, J=6.8 Hz, 1H), 4.84 (dd, J=7.2, 10.8 Hz, 1H), 5.05-5.14 (m, 3H), 6.00 (d, J=9.4 Hz, 1H), 7.30-7.35 (m, 5H). ES-MS (m/z): 911.5 (M+H)⁺.

Step 2, as shown in FIG. 30 and described below 9-benzyloxycarbonylamino-2′-acetoxy-4″-aminoethylcarbamate erythromycin (“237”) was sythesized as follows: To a solution of 236 (15 mg, 0.016 mmol) in toluene (0.8 mL) and dichloroethane (0.2 mL) was added potassium carbonate (11 mg, 0.080 mmol) and 1,1′-carbonyldiimidazole (4.8 mg, 0.030 mmol). The resulting mixture was stirred at 45° C. for 2h, and ethylenediamine (40 μL, 0.60 mmol) was added. The mixture was continually stirred at the same temperature for 1 h and diluted with EtOAc/H₂O. The separated organic layer was washed with water, brine, and dried over Na₂SO₄. Condensation afforded 19 mg of a white solid 237, which is about 80% pure by LC/MS and can be subjected to the next step directly without further purification. Compound 237: TLC (10% MeOH/CH₂Cl₂) R_{f=}0.08. ES-MS (m/z): 997.5 (M+H)⁺.

Step 3, as shown in FIG. 30 and described below, the probe 9-benzyloxycarbonylamino-4″-Bodipy FL aminoethylcarbamate erythromycin (“238”) was synthesized as follows: To a solution of 237 (7.0 mg, 0.007 mmol) in DMF (0.3 mL) was added a solution of Bodipy FL SE (2.5 mg, 0.006 mmol). The reactant was stirred at r.t. for 2 h. After removal of solvent under vacuum, the residue was purified by PTLC with 10% MeOH/CH₂Cl₂ and afforded 5.4 mg of an orange solid. The orange solid was dissolved in methanol (0.6 mL), stirred at r.t. overnight. The reactant was subject directly to PTLC. purification to give 0.9 mg (11%) of the desired product (“238”) as an orange solid. The intermediate with the acetoxy group shows: TLC (10% MeOH/CH₂Cl₂) R_{f=}0.48. ¹H NMR (400 MHz, CDCl₃): δ (ppm) 0.89 (t, J=7.2 Hz, 3H), 0.93-1.00 (m, 4H), 1.06-1.38 (m, 24H), 1.45-1.58 (m,2H), 1.65 (dd, J=4.4, 14.4 Hz, 1H), 1.66-1.75 (m, 3H), 1.82-1.94 (m, 2H), 2.03 (s, 3H), 2.14-2.18 (m, 1H), 2.27 (s, 3H), 2.28 (s, 3H), 2.36-2.43 (m, 2H), 2.57 (s, 3H), 2.63 (t, J=7.2 Hz, 2H), 2.70 (app t, J=6.8 Hz, 1H), 2.82 (app t, J=6.8 Hz, 2H), 3.23-3.40 (m, 6H), 3.34 (s, 3H), 3.56 (d, J=6.4 Hz, 1H), 3.68-3.74 (m, 2H), 4.16-4.24 (m, 2H), 4.53 (dd, J=3.6, 9.6 Hz, 1H), 4.61 (d, J=9.6 Hz, 1H), 4.69 (dd, J=3.6, 21.2 Hz, 1H), 4.79-4.84 (m, 2H), 4.92 (dd, J=3.6, 9.2 Hz, 1H), 5.05-5.16 (m, 3H), 5.48 (br s, 1H), 6.01 (d, J=9.2 Hz, 1H), 6.06 (br s, 1H), 6.14 (s, 1H), 6.25 (d, J=4.0 Hz, 1H), 6.88 (d, J=4.0 Hz, 1H), 7.10 (s, 1H), 7.30-7.34 (m, 5H). ES-MS (m/z): 1271.6 (M+H)⁺. Compound 238: TLC (10% MeOH/CH₂Cl₂) R_{f}=0.10. ¹H NMR (400 MHz, CDCl₃): δ (ppm) 0.89 (t, J=7.2 Hz, 3H), 1.07-1.34 (m, 24H), 1.45-1.56 (m, 3H), 1.65 (dd, J=4.4, 14.4 Hz, 1H), 1.66-1.75 (m, 5H), 1.86-1.94 (m, 3H), 2.16-2.20 (m, 1H), 2.27 (s, 6H), 2.39 (s, 3H), 2.35-2.43 (m, 3H), 2.56 (s, 3H), 2.65 (t, J=7.2 Hz, 2H), 2.84-2.89 (m, 2H), 3.25-3.36 (m, 8H), 3.31 (s, 3H), 3.72-3.78 (m, 3H), 4.16-4.24 (m, 2H), 4.55 (d, J=9.6 Hz, 1H), 4.62 (d, J=9.6 Hz, 1H), 5.07 (br s, 1H), 5.09 (s, 2H), 6.05 (d, J=9.2 Hz, 1H), 6.13 (s, 1H), 6.28 (br s, 1H), 6.89 (d, J=4.0 Hz, 1H), 7.10 (s, 1H), 7.30-7.34 (m, 5H).

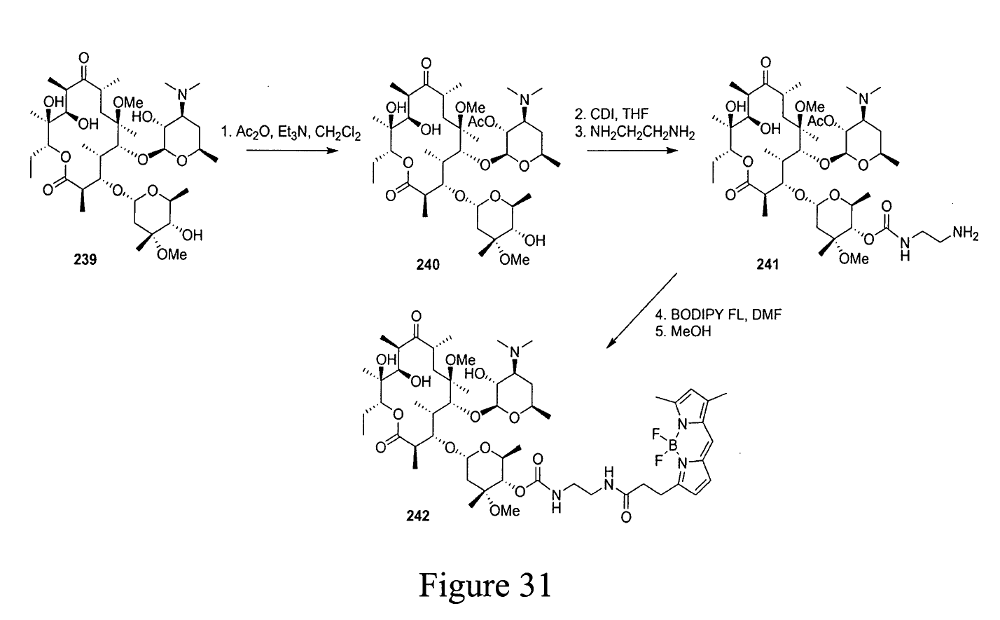

Another macrolide probe of this invention is illustrated in FIG. 31 and described below. Step 1 in the preparation of 2′-acetoxy-clarithromycin (“240”) is synthesized as follows: To a solution of clarithromycin (“239”) (49 mg, 0.065 mmol) in CH₂Cl₂ (0.8 mL) was added triethylamine (25 μL, 0.18 mmol) and acetic anhydride (9.0 μL, 0.089 mmol) and the reaction mixture was stirred at r.t. overnight. The reaction solution was diluted with EtOAc/H₂O, and the separated organic layer was washed with brine and dried over Na₂SO₄. Condensation afforded 51 mg of a white solid 240. Compound 240: TLC (10% MeOH/CH₂Cl₂) R_{f}=0.42. ¹H NMR (400 MHz, CDCl₃): δ (ppm) 0.84 (t, J=7.2 Hz, 3H), 0.93 (d, J=7.6 Hz, 3H), 1.12 (d, J=6.0 Hz, 3H), 1.13 (s, 3H), 1.14 (d, J=6.4 Hz, 3H), 1.21 (d, J=8.0 Hz, 3H), 1.23 (d, J=6.4 Hz, 3H), 1.28 (s, 3H), 1.30 (d, J=6.0 Hz, 3H), 1.38 (s, 3H), 1.44-1.50 (m, 1H), 1.58-1.74 (m, 5H), 1.84-1.96 (m, 2H), 2.06 (s, 3H), 2.17 (d, J=10.0 Hz, 1H), 2.26 (s, 6H), 2.36 (d, J=15.2 Hz, 1H), 2.55-2.63 (m, 2H), 2.83-2.88 (m, 1H), 2.97 (app q, J=6.8 Hz, 1H), 3.02 (s, 3H), 3.06 (d, J=9.6 Hz, 1H), 3.21 (s, 1H), 3.37 (s, 3H), 3.45-3.50 (m, 1H), 3.61 (d, J=8.0 Hz, 1H), 3.75 (s, 1H), 3.76 (d, J=8.8 Hz, 1H), 3.95-4.01 (m, 1H), 3.99 (s, 1H), 4.67 (d, J=7.6 Hz, 1H), 4.75 (dd, J=7.2, 10.8 Hz, 1H), 4.94 (d, J=4.8 Hz, 1H), 5.06 (dd, J=2.0, 10.8 Hz, 1H). ES-MS (m/z): 790.4 (M+H)⁺.

Step 2, as illustrated in FIG. 31 and described below, 2′-acetoxy-4″-aminoethylcarbamate clarithromycin (“241”) is synthesized as follows: To a solution of 240 (51 mg, 0.065 mmol) in toluene (1.8 mL) and dichloroethane (0.2 mL) was added potassium carbonate (23 mg, 0.17 mmol) and 1,1′-carbonyldiimidazole (13 mg, 0.080 mmol). The resulting mixture was stirred at 35° C. overnight, and ethylenediamine (220 μL, 3.3 mmol) was added. The mixture was stirred at 45° C. for 1 h and diluted with EtOAc/H₂O. The separated organic layer was washed with water, brine, and, dried over Na₂SO₄. Condensation afforded 54 mg of a white solid 241, which is about 80% pure by LC/MS and was subjected to the next step directly without further purification. Compound 241: TLC (10% MeOH/CH₂Cl₂) R_{f}=0.08. ¹H NMR (400 MHz, CDCl₃): δ (ppm) 0.84 (t, J=7.6 Hz, 3H), 0.94 (d, J=7.2 Hz, 3H), 1.11-1.14 (3 lines m, 9H), 1.18-1.22 (5 lines m, 12 H), 1.27-1.32 (m, 1H), 1.36 (s, 3H), 1.46-1.52 (m, 1H), 1.58-1.73 (m, 4H), 1.86-1.96 (m, 2H), 2.05 (s, 3H), 2.27 (d, J=8.0 Hz, 1H), 2.29 (s, 6H), 2.41 (d, J=15.2 Hz, 1H), 2.54-2.58 (m, 1H), 2.73 (dt, J=4.0, 11.2 Hz, 1H), 2.81-2.89 (m, 3H), 2.99 (app q, J=6.8 Hz, 1H), 3.02 (s, 3H), 3.21 (br s, 1H), 3.26 (app q, J=6.0 Hz, 2H), 3.36 (s, 3H), 3.61 (d, J=7.6 Hz, 1H), 3.66-3.71 (m, 1H), 3.74 (s, 1H), 3.76 (d, J=8.8 Hz, 1H), 3.99 (s, 1H), 4.25-4.29 (m, 1H), 4.54 (d, J=10.0 Hz, 1H), 4.66 (d, J=7.2 Hz, 1H), 4.76 (dd, J=7.2, 11.2 Hz, 1H), 4.98 (d, J=4.8 Hz, 1H), 5.07 (dd, J=2.0, 11.2 Hz, 1H), 5.17 (app t, J=5.2 Hz, 1H), 7.18 (dd, J=2.8, 7.6 Hz, 1H). ES-MS (m/z): 876.4 (M+H)⁺.

Step 3, as illustrated in FIG. 31 and described below, the Probe 4″-Bodipy FL-aminoethylcarbamate clarithromycin (“242”) is synthesized as follows: to a solution of 241 (12.0 mg, 0.014 mmol) in DMF (0.3 mL) was added a solution of Bodipy FL SE (2.5 mg, 0.006 mmol) in 0.2 mL DMF. The mixture was stirred at r.t. for 1 h. After removal of solvent under vacuum, the residue was purified by PTLC with 10% MeOH/CH₂Cl₂ to give an orange solid (4.8 mg). The orange solid was dissolved in methanol (0.6 mL), stirred at r.t. overnight and then at 60° C. for 1 h. The mixture was subjected to PTLC. purification to give 1.6 mg (24%) of the desired product as an orange solid. The intermediate with acetoxy group: TLC (10% MeOH/CH₂Cl₂) R_{f}=0.52. ES-MS (m/z): 1150.5 (M+H)⁺. Compound 242: TLC (10% MeOH/CH₂Cl₂) R_{f}=0.10. ¹H NMR (400 MHz, CDCl₃): δ (ppm) 0.85 (t, J=7.6 Hz, 3H), 1.09 (d, J=8.0 Hz, 3H), 1.12-1.23 (8 lines m, 21H), 1.26-1.31 (m, 2H), 1.38 (s, 3H), 1.46-1.52 (m, 1H), 1.63 (dd, J=4.8, 11.2 Hz, 1H), 1.68 (br s, 1H), 1.79-1.88 (m, 2H), 1.90-1.97 (m 2H), 2.27 (s, 3H), 2.40 (d, J=14.8 Hz, 1H), 2.48 (br s, 6H), 2.57 (s, 3H), 2.55-2.61 (m, 1H), 2.64 (t, J=7.6 Hz, 1H), 2.86-2.93 (m, 2H), 2.98 (app q, J=7.2 Hz, 1H), 3.04 (s, 3H), 3.20 (s, 1H), 3.26 (t, J=8.0 Hz, 2H), 3.31 (s, 3H), 3.32-3.30 (m, 4H), 3.66 (d, J=6.8 Hz, 1H), 3.76 (s, 1H), 3.77 (d, J=8.8 Hz, 1H), 3.98 (s, 1H), 4.25-4.29 (m, 1H), 4.52 (d, J=9.2 Hz, 1H), 4.61 (br s, 1H), 4.97 (d, J=5.2 Hz, 1H), 5.07 (dd, J=2.0, 11.6 Hz, 1H), 5.53 (br s,1H), 6.14 (s, 1H), 6.18 (br s, 1H), 6.27 (d, J=4.0 Hz, 1H), 6.89 (d, J=3.6 Hz, 1H), 7.10 (s, 1H). ES-MS (m/z): 1108.5 (M+H)⁺.

### Example III

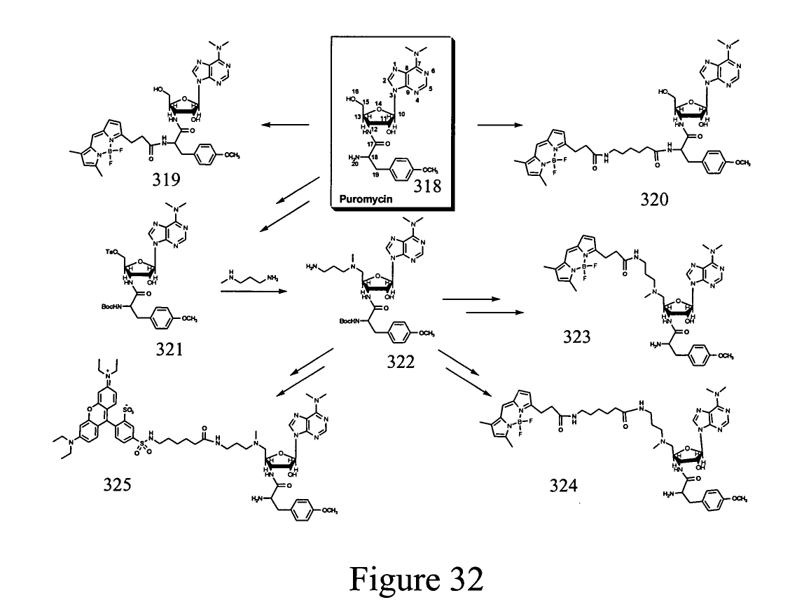

Puromycin probes: Another series of probes of this invention are based on Puromycin. FIG. 32 illustrates the synthesis necessary to prepare the probe 20-Bodipy FL puromycin (“319”): To a solution of puromycin 318 (4.8 mg, 0.009 mmol) in DMF (0.07 mL) was added triethylamine (4 μL, 0.029 mmol) and Bodipy FL SE (2.7 mg, 0.007 mmol). The resulting mixture was stirred at r.t. overnight. After removal of the solvent under vacuum, the residue was purified by PTLC with 10% MeOH/CH₂Cl₂ to afford an orange solid 319 (2.0 mg, 41%). ES-MS (m/z): 746 (M+H)⁺.

As illustrated in FIG. 32 and described below, the probe 20-Bodipy FL-X puromycin (“320”) was synthesized as follows: To a stirred solution of BODIPY FL-X, SE (0.7 mg, 0.0014 mmol) in 0.15 mL anhydrous DMF at room temperature, was added puromycin (5 mg, 0.0092 mmol). The mixture was allowed to stir for two days, most of the starting material remained intact. Triethylamine (1 drop) was then added, and the resulting mixture was allowed to stir at room temperature for 18 hrs. The solvent was removed and the crude product was purified by PTLC (dichloromathane:methanol 1:4 Rf: 0.3) to afford 20-N-BODIPY FL-X puromycin (0.8 mg, 66%) as a reddish film. MS (M+H)⁺, 858.3. ¹H NMR (400 MHz, CD₃OD): δ=8.39 (s, 1H), 8.20 (s, 1H), 8.19 (d, J=10.4Hz, 1H), 7.98 (d, J=10.4 Hz, 1H), 7.40 (s, 1H), 7.16 (d, J=8.8Hz, 2H), 6.97 (d, J=3.6 Hz, 1H), 6.84 (d, J=8.8 Hz, 2H), 6.28 (d, J=4.0Hz, 1H), 6.20 (s, 1H), 5.95 (s, 1H), 4.61-4.54 (m, 3H), 3.98 (m, 1H), 3.80 (dd, 1H), 3.75 (s, 3H), 3.56-3.45 (m, 5H), 3.18 (q, J=6.8 Hz, 3H), 3.11 (t, J=6.8 Hz, 2H), 3.04-2.99 (m, 2H), 2.91-2.83 (m, 2H), 2.56 (t, J=7.2 Hz, 2H), 2.39 (s, 3H), 2.27 (s, 3H), 2.16 (t, J=7.6 Hz, 2H), 1.52 (m, 2H), 1.43 (m, 2H), 1.31 (ts, J=7.6 Hz, 6H), 1.21 (q, J=6.8 Hz, 2H), 0.89 (m, 2H).

As illustrated in FIG. 32 and described below, probe 323 was synthesized as follows: Step 1, to a solution of puromycin 318 (20.0 mg, 0.037 mmol) in DMF/H₂O (0.32 mL/0.08 mL) was added triethylamine (20 μL, 0.143 mmol) and di-t-butyl bicarbonate (8.5 mg, 0.039 mmol). The resulting mixture was stirred at 60° C. for 2.5 h and diluted with EtOAc/H₂O. The organic layer was washed with H₂O, brine, dried over Na₂SO₄ and condensed to afford a white solid, which was dissolved again in pyridine (0.5 mL) and tosyl chloride (10.0 mg, 0.052 mmol) was added. After the mixture was stirred at r.t. overnight, the mixture was diluted with EtOAc/H₂O. The organic layer was washed with brine, dried over Na₂SO₄ and condensed. PTLC. purification with 5% MeOH/CH₂Cl₂ afforded a white solid (23 mg, 85% yield overall two steps). Compound 321: TLC (5% MeOH/CH₂Cl₂) R_{f}=0.50. ¹H NMR (400 MHz, CDCl₃): δ (ppm) 1.41 (s, 9H), 2.41 (s, 3H), 2.92 (dd, J=8.0, 13.6 Hz, 1H), 3.03 (dd, J=6.8, 13.6 Hz, 1H), 3.55 (br s, 6H), 3.75 (s, 3H), 4.20 -4.36 (m, 4H), 4.44 (dd, J=4.4, 6.8 Hz, 1H), 5.09 (d, J=6.0 Hz, 1H), 5.56 (d, J=4.4 Hz, 1H), 6.33 (br s, 1H), 6.85 (d, J=8.8 Hz, 2H), 7.10 (d, J=8.8 Hz, 2H), 7.28 (d, J=8.0 Hz, 2H), 7.73 (d, J=8.0 Hz, 2H), 7.74 (s, 1H), 8.20 (s, 1H), 8.63 (br s, 1H).

Step 2, as illustrated in FIG. 32 and described below, 20-Boc-16-N-methylpropanediamino-puromycin (“322”) was synthesized as follows: To a solution of 321 (46 mg) in N-methylpropanediamine (1.5 mL) was stirred at r.t. overnight. After removal of solvent under vacuum, the residue was purified by PTLC with 6% MeOH/CH₂Cl₂ to afford a white solid 322 (18 mg). Compound 322: TLC (6% MeOH/CH₂Cl₂) R_{f}=0.15. ¹H NMR (400 MHz, CD₃OD): δ (ppm) 1.40 (s, 9H), 1.74 (t, J=6.4 Hz, 2H), 2.23 (s, 3H), 2.45 (d, J=15.2 Hz, 1H), 2.52-2.62 (m, 2H), 2.85 (dd, J=8.0, 15.2 Hz, 1H), 2.96-3.02 (m, 2H), 3.51 (br s, 6H), 3.77 (s, 3H), 4.04-4.07 (m, 2H), 4.29 (t, J=7.2 Hz, 1H), 4.55-4.62 (m, 3H), 5.98 (d, J=1.6 Hz, 1H), 6.86 (d, J=8.8 Hz, 2H), 7.19 (d, J=8.8 Hz, 2H), 8.19 (s, 1H), 8.25 (s, 1H). ES-MS (m/z): 642.3(M+H)⁺.

Step 3, as illustrated in FIG. 32 and described below, 16-N-Bodipy FL-N-methylpropanediamino-puromycin (“323”) was synthesized as follows: To a solution of 322 (1.0 mg, 0.001 mmol) in DMF (0.10 mL) was added Bodipy FL SE (0.5 mg, 0.001 mmol). The reactant was stirred at r.t. overnight. After removal of the solvent under vacuum, the residue was purified by PTLC with 5% MeOH/CH₂Cl₂ to afford 0.7 mg of an orange solid. The orange solid was then dissolved in 0.1 mL CH₂Cl₂ and HCl ether solution (2.0 M, 5 μL) was added. After stirring at r.t. for 20 min, the mixture was purified by PTLC. to afford 0.4 mg (32%) of an orange solid. Compound 323: TLC (20% MeOH/CH₂Cl₂) R_{f}=0.25. ¹H NMR (400 MHz, CD₃OD): δ (ppm) 1.64 (t, J=7.2 Hz, 2H), 2.25 (s, 3H), 2.27 (s, 3H), 2.46-2.54 (m, 4H), 2.49 (s, 3H), 2.58-2.70 (m, 2H), 2.79-2.83 (4 lines m, 2H), 2.90-2.94 (4 lines m, 2H), 3.13-3.17 (m, 4H), 3.48 (br s, 6H), 3.68 (t, J=8.0 Hz, 1H), 3.76 (s, 3H), 4.05-4.09 (m, 2H), 4.46-4.52 (m, 3H), 5.97 (d, J=1.2 Hz, 1H), 6.20 (s, 1H), 6.23 (d, J=4.0 Hz, 1H), 6.85 (d, J=8.8 Hz, 2H), 6.94 (d, J=4.0 Hz, 1H), 7.14 (d, J=8.8 Hz, 2H), 7.38 (s, 1H), 8.16 (s, 1H), 8.20 (s, 1H). ES-MS (m/z): 816.4 (M+H)⁺.

As illustrated in FIG. 32 and described below, 16-N-Rhodamine Red-N-methylpropanediamino-puromycin (“324”) was synthesized as follows: To a solution of 322 (1.5 mg, 0.002 mmol) in DMF (0.16 mL) was added Rhodamine Red SE (1.0 mg, 0.001 mmol). The reactant was stirred at r.t. overnight. After removal of the solvent under vacuum, the residue was purified by PTLC with 15% MeOH/CH₂Cl₂ to afford 1.2 mg of a red solid. The red solid was then dissolved in CH₂Cl₂/THF (0.1 mL/0/1 mL) and HCl ether solution (2.0 M, 30 μL) was added. After stirring at r.t. for 30 min, direct PTLC purification afforded 0.4 mg of a red solid. Intermediate with Boc group: TLC (15% MeOH/CH₂Cl₂) R_{f}=0.20. ¹H NMR (400 MHz, CD₃OD): δ (ppm) 1.29 (t, J=7.2 Hz, 12H), 1.38 (s, 9H), 1.44-1.55 (m, 4H), 1.62-1.67 (m, 2H), 2.30 (s, 3H), 2.52-2.60 (m, 3H), 2.74-2.86 (m 2H), 2.96-3.03 (m, 4H), 3.12-3.17 (m, 2H), 3.48 (br s, 6H), 3.65 (q, J=7.2 Hz, 8H), 3.75 (s, 3H), 4.09-4.13 (m, 2H), 4.28 (t, J=7.6 Hz, 1H), 4.50-4.57 (m, 2H), 5.99 (s, 1H), 6.84 (d, J=8.8 Hz, 2H), 6.92 (s, 2H), 6.94-6.99 (m, 2H), 7.09 (dd, J=3.2, 9.6 Hz, 2H), 7.17 (d, J=8.8 Hz, 2H), 7.51 (d, J=7.6 Hz, 1H), 8.09 (dd, J=2.0, 8.4 Hz, 1H), 8.19 (s, 1H), 8.22 (s, 1H), 8.65 (d, J=2.0 Hz, 1H). Compound 324: TLC (20% MeOH/CH₂Cl₂) R_{f}=0.15. ¹H NMR (400 MHz, CD₃OD): δ (ppm) 1.29 (t, J=7.2 Hz, 12H), 1.45-1.49 (m, 2H), 1.51-1.58 (m, 2H), 1.73-1.77 (m, 2H), 2.01-2.09 (m, 3H), 2.14 (app t, J=7.6 Hz, 2H), 2.59 (s, 3H), 2.82-2.88 (m, 2H), 3.00-3.21 (m, 6H), 3.48 (m, 6H), 3.66 (q, J=7.2 Hz, 8H), 3.78 (s, 3H), 4.01-4.08 (m, 2H), 4.21 (app t, J=7.6 Hz, 1H), 4.64-4.67 (m, 2H), 6.00 (s, 1H), 6.93 (d, J=2.0 Hz, 2H), 6.91-6.98 (m, 2H), 7.09 (d, J=9.6 Hz, 2H), 7.23 (d, J=8.8 Hz, 2H), 7.52 (d, J=8.0 Hz, 2H), 7.70 (d, J=8.0 Hz, 1H), 8.10 (dd, J=2.0, 8.0 Hz, 1H), 8.16 (s, 1H), 8.23 (s, 1H), 8.66 (d, J=1.2 Hz, 1H).

As illustrated in FIG. 32 and described below, 16-N-Bodipy FL-X-N-methylpropanediamino-puromycin (“325”) was synthesized as follows: To the solution of 322 (1.5mg, 0.002 mmol) in DMF (0.16mL) was added Bodipy FL-X SE (0.8 mg, 0.001 mmol). The reactant was stirred at r.t. overnight. After removal of solvent under vacuum, the residue was purified by PTLC with 6% MeOH/CH₂Cl₂ and afforded 1.0 mg of an orange solid. The orange solid was then dissolved in 0.15 mL TFA and stirred at r.t. for 4 min. After removal of the solvent under vacuum, direct PTLC. purification afforded 0.6 mg (46%) of an orange solid. Intermediate with Boc group: TLC (6% MeOH/CH₂Cl₂) R_{f=}0.38. ¹H NMR (400 MHz, CD₃OD): δ (ppm) 1.23-1.29 (m, 2H), 1.39 (s, 9H), 1.45 (t, J=7.6 Hz, 2H), 1.54 (t, 7.6 Hz, 2H), 1.65 (t, J=7.6 Hz, 2H), 2.09 (td, J=3.6, 7.2 Hz, 2H), 2.27 (s, 3H), 2.29 (s, 3H), 2.50 (s, 3H), 2.58 (app t, J=7.6 Hz, 4H), 2.74-2.85 (m, 2H), 2.99 (dd, J=2.8, 13.6 Hz, 1H), 3.12-3.22 (m, 8H), 3.48 (br s, 6H), 3.76 (s, 3H), 4.08-4.12 (m, 1H), 4.29 (t, J=7.6 Hz, 1H), 4.50-4.56 (m, 3H), 6.00(s, 1H), 6.20 (s, 1H), 6.30 (d, J=4.0 Hz, 1H), 6.85 (d, J=8.8 Hz, 2H), 6.99 (d, J=4.0 Hz, 1H), 7.17 (d, J=8.8 Hz, 2H), 7.41 (s, 1H), 8.20 (s, 1H), 8.22 (s, 1H). Compound 325: TLC (20% MeOH/CH₂Cl₂) R_{f}=0.24. ¹H NMR (400 MHz, CD₃OD): δ (ppm) 1.24-1.30 (m, 2H), 1.44-1.48 (m, 2H), 1.55 (t, J=7.6 Hz, 2H), 1.61-1.66 (m, 2H), 2.07-2.12(m, 2H), 2.26 (s, 3H), 2.28 (s, 3H), 2.44-2.49 (m, 2H), 2.50 (s, 3H), 2.57 (app t, J=7.6 Hz, 2H), 2.65-2.72 (m, 1H), 2.83-2.87 (m, 1H), 2.92-2.96 (m, 1H), 3.12-3.22 (m, 8H), 3.48 (br s, 6H), 3.67-3.70 (m, 1H), 3.76 (s, 3), 4.08 (t, J=7.6 H, 1H), 4.48-4.54 (m, 3H), 5.99 (d, J=1.6 Hz, 1H), 6.22 (s, 1H), 6.34 (d, J=4.0 Hz, 1H), 6.87 (d, J=8.8 Hz, 2H), 7.01 (d, J=4.0 Hz, 1H), 7.16 (d, J=8.8 Hz, 2H), 7.43 (s, 1H), 8.20 (s, 1H), 8.23 (s, 1H). ES-MS (m/z): 929.6 (M+H)⁺.

### Example IV

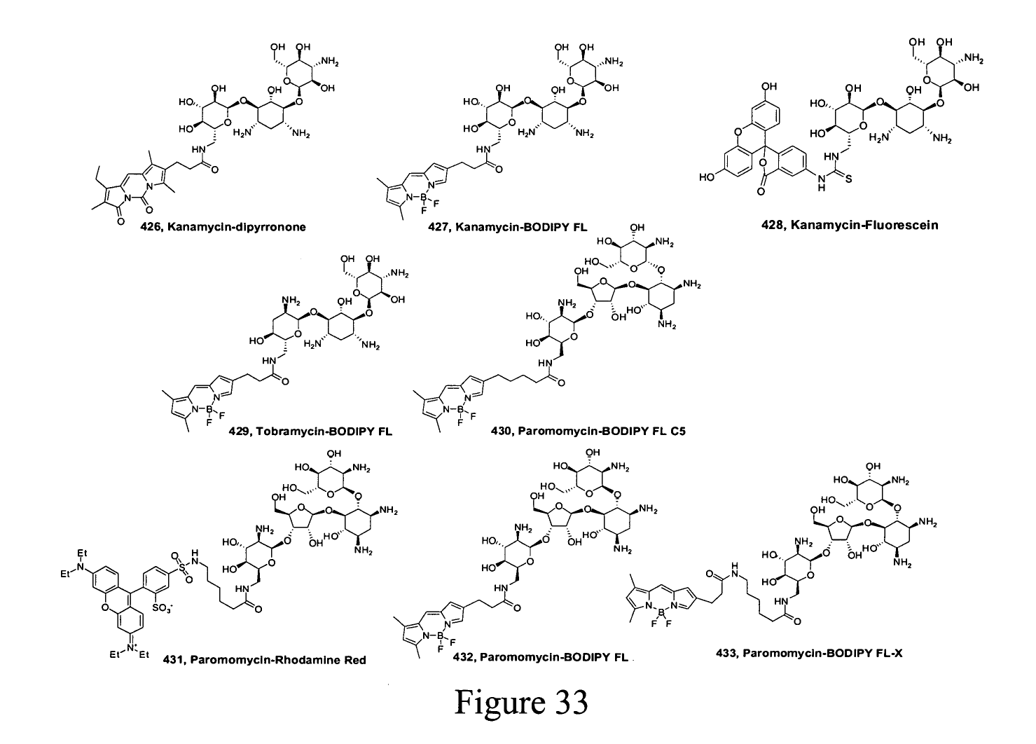

Aminoglycoside Probes: Another series of probes of this invention are based on aminoglycoside, and illustrated in FIG. 33. The general procedure for an aminoglycoside probe comprises: To a solution of kanamycin sulfate (8.2 mg, 0.014 mmol) in H₂O (0.24 mL) was added a solution of dipyrrinone SE (Justin O. Brower; David A. Lightner J. Org. Chem. 2002, 67, 2713-1716) (1.1 mg, 0.003 mmol) in DMF (0.12 mL). The resulting solution was stirred at r.t. overnight, and diluted with 0.2 mL H₂O to make it clear. The reaction solution was purified by HPLC on ODS column with a gradient of acetonitrile and water. The acetonitrile concentration was increased from 0% to 40% over 30 min. All solvents contain 1% trifluoroacetic acid. After concentration, 0.7 mg (34%) of a yellow solid-single isomer was isolated. Compound 426: ¹H NMR (400 MHz, CD₃OD): δ (ppm) 0.82 (t, J=7.6 Hz, 3H), 1.52 (s, 3H), 1.78 (s, 3H), 1.98 (app t, J=7.6 Hz, 2H), 2.12-2.15 (m, 1H), 2.21 (s, 3H), 2.22-2.26 (m, 2H), 2.30-2.35 (m, 1H), 2.40 (t, J=8.4 Hz, 1H), 2.56-2.61 (m, 2H), 2.80 (dd, J=3.6, 9.6 Hz, 1H), 2.85 (dd, J=3.2, 14.0 Hz, 1H), 2.94-2.97 (m, 1H), 3.07-3.25 (m, 8H), 3.32 (app d, J=9.6 Hz, 1H), 3.39 (dd, J=3.6, 10.0 Hz, 1H), 3.48 (app t, J=8.0 Hz, 2H), 3.52 (app d, J=11.6 Hz, 1H), 4.67 (d, J=4.0 Hz, 1H), 4.70 (d, J=3.6 Hz, 1H), 6.46 (s, 1H). ES-MS (m/z): 795.3 (M+H)⁺.

Kanamycin-Bodipy FL (“427”) (1.4 mg, 38%) in FIG. 33 has a similar preparation as described for compound 426. Compound 427: ES-MS (m/z): 742.5 (M+H)⁺.

Kanamycin-Fluorescein (“428”) (1.2 mg, 20%) in FIG. 33 has a similar preparation as described for compound 426. Compound 428: ES-MS (m/z): 874.1 (M+H)⁺.

Tobramycin-Bodipy FL (“429”) (0.5 mg, 24%) in FIG. 33 has a similar preparation as described for compound 426. Compound 429: ES-MS (m/z): 742.4 (M+H)⁺.

Paromomycin-Bodipy FL-X (“430”) (0.5 mg, 23 %) in FIG. 33 has a similar preparation as described for compound 426. Compound 430: ES-MS (m/z): 914.4 (M+H)⁺.

Paromomycin Rhodamine Red (“431”) (0.5 mg, 61%) in FIG. 33 has a similar preparation as described for compound 426. Compound 431: ¹H NMR (400 MHz, CD₃OD): δ (ppm) 1.30 (t, J=6.8 Hz, 12H), 1.37 (app q, J=7.0 Hz, 2H), 1.48 (app q, J=7.0 Hz, 2H), 1.61 (app q, J=7.0 Hz, 2H), 1.84 (q, J=12.8 Hz, 2H), 2.23 (t, J=6.8 Hz, 2H), 2.40-2.44 (m, 1H), 3.09 (t, J=6.8 Hz, 2H), 3.09-3.13 (m, 1H), 3.25-3.37 (m, 2H), 3.41 (d, J=8.4 Hz, 1H), 3.48-3.63 (m, 5H), 3.68 (q, J=6.8 Hz, 8H), 3.76-3.98 (m, 8H), 4.10-4.12 (m, 1H), 4.13-4.14 (m, 1H), 4.31-4.33 (m, 1H), 4.45 (app t, J=5.6 Hz, 1H), 5.15(s, 1H), 5.32 (s, 1H), 5.59 (d, J=4.0 Hz, 1H), 6.96 (s, 2H), 6.98-7.02 (m, 2H), 7.08 (d, J=12.8 Hz, 1H), 7.11 (d, J=12.8 Hz, 1H), 7.56 (d, J=8.0 Hz, 1H), 8.13 (d, J=8.0 Hz, 1H), 8.64 (s, 1H).

Paromomycin-Bodipy FL (“432”) (0.8 mg, 43%) in FIG. 33 has a similar preparation as described for compound 426. Compound 432: ES-MS (m/z): 890.4 (M+H)⁺.

Paromomycin-Bodipy FL-X (“433”) in FIG. 33 has a similar preparation as described for compound 426. Compound 433: ES-MS (m/z): 1003.5 (M+H)⁺.

### Example V

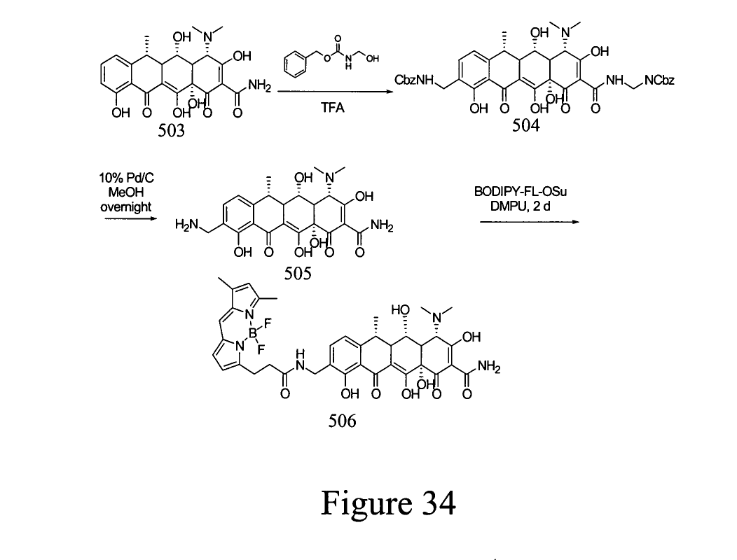

Tetracycline Probes: Another series of probes of this invention are based on tetracycline. The general procedure for a tetracycline probe is illustrated in FIG. 34 and described below. The {9-[(benzyloxycarbonylamino-methyl)-carbamoyl]-7-dimethylamino-1,6,8,10a,11-pentahydroxy-5-methyl-10,12-dioxo-5,5a,6,6a,7,10,10a,12-octahydro-naphthacene-2-ylmethyl}-carbamic acid benzyl ester (“504”) is synthesized as follows: Step 1 to a solution of doxycycline 503 (100 mg, 0.2 mmol) in trifluoroacetic acid (1 mL) was added benzyl N-(hydoxymethyl)carbamate (200 mg, 1.1 mmol) and stirred at r.t. overnight. The reaction mixture was triturated with ether, filtered and washed with ether to give 160 mg of the desired crude light yellow solid. This solid was used for the next reaction without further purification. Compound 504: MS(M +H)⁺771; ¹H NMR (400 MHz, CD₃OD) δ 7.62 (d, J=8.0 Hz,1H), 7.40-7.32 (m, 10 H), 7.06 (d, J=7.6 Hz, 1H), 5.17 (s, 2H), 5.16 (s, 2H), 4.57 (s, 1H), 4.42 (s, 2H), 4.16 (s, 2H), .58-3.56 (m, 1H), 2.94 (br s, 6H), 2.94-2.81 (m, 2H), 2.63-2.58 (m, 1H), 1.56 (d, J=6.8 Hz, 3H).

Step 2, as illustrated in FIG. 34 and described below, 9-aminomethyl doxycycline; 9-aminomethyl-4-dimethylamino-3,5,10,12,12a-pentahydroxy-6-methyl-1,11-dioxo-1,4,4a,5,5a,6,11,12a-octahydro-naphthacene-2-carboxylic acid amide (“505”) is synthized as follows: A heterogeneous solution of CBZ (benzyloxycarbonyl) protected aminomethyl doxycycline 504 (20 mg, 0.025 mmol) in MeOH (1 mL) and 10% Pd/C (20 mg) was stirred at r.t. overnight under hydrogen balloon. The reaction mixture was filtered and the solvent of the filtrate was removed under reduced pressure. The residue was purified by HPLC on ODS column with a gradient of acetonitrile and water to give 5.5 mg of the desired 9-aminomethyl doxycycline 505 in 42% yield. The acetonitrile concentration was increased from 0% to 100% over 30 min. All solvents contain 1% trifluoroacetic acid. Compound 505: MS (M+H)⁺608; ¹H NMR (400 MHz, CD₃OD) δ 7.61 (d, J=8.0 Hz,1H), 7.06 (d, J=8.0 Hz, 1H), 4.20 (s, 1H), 4.16 (s, 2 H), 3.58 (dd, J=11.2 Hz and 8.8 Hz, 1H), 2.94 (br s, 3H), 2.94-2.69 (m, 2H), 2.89 (s, 3H), 2.58 (dd, J=12.0 Hz and 8.4 Hz, 1H), 1.56 (d, J=6.8 Hz, 3H).

Step 3, as illustrated in FIG. 34 and described below, 9-N-BODIPY-FL aminomethyl-doxycycline; 9-(4,4-difluoro-5,7-dimethyl-4-bora-3a,4a-diaza-s-indacene-3-propionyl)-aminomethyl-doxycycline (“506”) was synthesized as follows: To a solution of 9-aminomethyl-doxycycline (5 mg, 0.01 mmol) in DMPU (N,N′-dimethylpropyleneurea) (0.4 mL) was added BODIPY FL SE (4,4-difluoro-5,7-dimethyl-4-bora-3a,4a-diaza-s-indacene-3-propionic acid succinimidyl ester) (1.5 mg) and stirred at r.t for 2 days. The reaction mixture was purified directly with HPLC on an ODS column with a gradient of acetonitrile and water to give a mixture of the desired probe 506 and hydrolyzed BODIPY FL (4,4-difluoro-5,7-dimethyl-4-bora-3a,4a-diaza-s-indacene-3-propionic acid). The acetonitrile concentration was increase from 0% to 100% over 30 min. All solvents contain 1% trifluoroacetic acid. The mixture of the desired probe 506 and hydrolyzed BODIPY FL was purified again with HPLC. to give the dark brown solid (0.5 mg, 17% based on the used amount of BODIPY FL, SE). MS (M+H)⁺748; ¹H NMR (400 MHz, CD₃OD) δ 7.42 (d, J=7.2 Hz,1H), 7.41 (s, 1H), 6.93 (d, J=4.0 Hz, 1H), 6.88 (d, J=7.2 Hz, 1H), 6.27 (d, J=3.6 Hz, 1H), 6.21 (s, 1H), 4.40 (s, 1H), 4.37 (d, J=4.8 Hz, 2H), 3.55 (dd, J=11.6 Hz and 8.4 Hz, 1H), 3.21 (t, J=8.0 Hz, 2H), 2.97 (br s, 3H), 2.91 (br s, 3H), 2.71 (s, 1H), 2.68 (t, J=7.6 Hz, 1H), 2.62 (t, J=8.0 Hz, 2H), 2.56-2.52 (m, 1H), 2.50 (s, 3H), 2.28 (s, 3H), 1.52 (d, J=6.8 Hz, 3H).

9-N-BODIPY FL-X-aminomethyl-doxycycline (507) has a similar preparation as described for compound 506.

### Example VI

Methods of Use: To illustrate the use of fluorescent probes and the substantial art in development and optimization of such probes, we are providing detailed experiments in binding, displacement, and high-throughput screening (HTS) based on the fluorescent probes.

Preparation of Ribosome: To obtain E. coli ribosomes in sufficient quantity for high-throughput screening, procedures similar to literature were followed (Blaha, G. et al. Methods in Enzymology, 2000, 317, 292-295). To obtain higher yield for HTS, log phase cells were harvested after growth in Terrific Broth (TB) to an OD₆₀₀ of 2 rather than growth to a log phase OD₆₀₀ of 0.5 in Luria-Bertani media (LB). Cells were resuspended in buffer A (20 mM Tris-HCl pH 7.5, 100 mM NH₄Cl, 10 mM MgCl₂, 0.5 mM EDTA, and 6 mM β-mercaptoethanol) at 2 ml g cells. The cells were pelleted by spinning 15 min at 5000 rpm in a GSA rotor, the wash removed, and the cells again resuspended in buffer A. The cells were lysed by 5-6 passages through a microfluidizer. The cell debri was removed by spinning twice at 16,000 rpm in an SS-34 rotor, carefully transferring the supernatant between spins. Twenty-five ml portions of the resultant S30 supernatant were pelleted overnight in an ultracentrifuge at 33,000 rpm through 35 ml cushions of buffer A lacking β-ME and containing a total of 500 mM NH₄Cl and 1.1 M sucrose. The supernatant was removed from the glassy ribosome pellet by pouring and inverting to drain. The pellet was rinsed briefly with resuspension buffer (50 mM Tris-HCl pH 7.5, 150 mM NH₄Cl, 5 mM MgCl₂, and 6 mM βME) to remove any debri. The ribosomes were resuspended by gently stirring 3-4 ml of resuspension buffer with the pellet for up to an hour, and quantified by measurement of OD₂₆₀. Activity of ribosomes purified from TB cultures was equivalent to that from LB cultures in multiple biochemical assays. Purification of ribosomes from S. aureus was similar except prior to microfluidizing the cells an additional one hour incubation was performed at 37° C. in the presence of 300 μg lysostaphin/g cells.

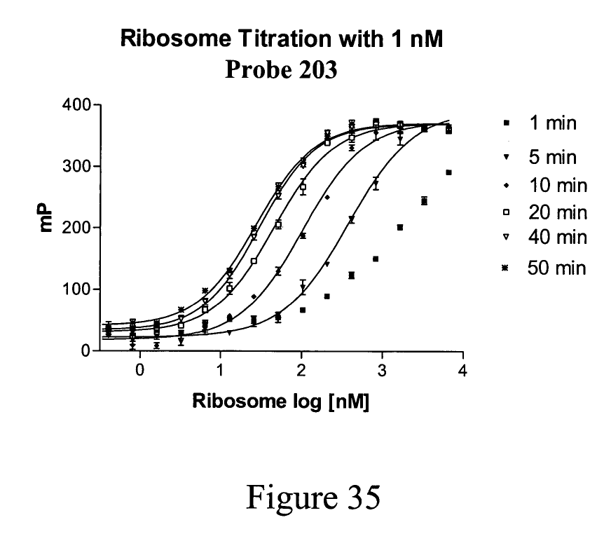

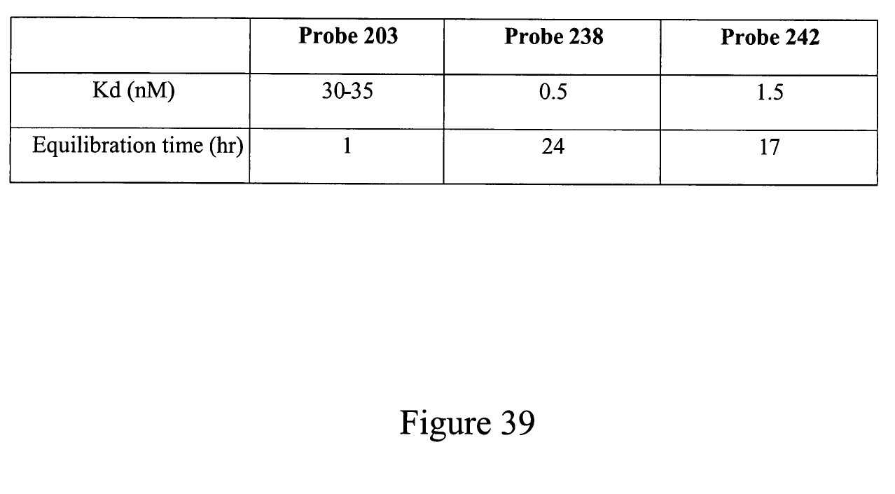

Determination of Probe Binding Affinity and Kinetics: To investigate uses of the fluorescently labeled probes we first had to accurately determine the binding constant of each of them to the 70S Ribosome. The binding affinity for said probes was initially checked in buffer reported in reference (Turconi, S. et al. J. Biomolecular Screening, 2001, 6, 275-290) containing: 20 mM Tris-HCl pH 7.5, 50 mM NH₄Cl, 10 mM MgCl₂, 0.05% Tween-20, and 20% Glycerol. The probe was titrated alone to see total fluorescent signal and probe concentrations were chosen that were at least 5-fold over background fluorescence from the buffer. The 70S ribosome was titrated over a range from the highest possible based on the prep concentration down to low nM values (1650 nM to 0.4 nM) across a small range of different probe concentrations. The fluorescence polarization was then read at various time points using a fluorescence polarization detector set for the appropriate fluorophore (for Bodipy FL it was set at 480 nM excitation and 535 nM emission) (see FIG. 35). In the ribosome titrations we were able to detect upwards of a 300 mP shift. This allowed us to determine a binding affinity for each probe and to set an appropriate concentration for subsequent competition experiments. As an example, the kinetics shown for Probe 203 indicate that it has reached equilibrium only after greater than a half hour incubation. Probe 238 and Probe 242 had greater affinity and even slower kinetics, as summarized in FIG. 39. Note that Probe 238 and Probe 242 have similar or slightly higher affinity for ribosomes than values reported in the literature for the parent erythromycin, while Probe 203 has a slightly reduced affinity. Based on these data, Probe 238 and Probe 242 offer high-affinity probes with the potential uses described above. For example, because the range of resolvable inhibitor potency is limited by the affinity of the fluorescent ligand (Huang, X. J. Biomolecular Screening, 2003, 8, 34-38), displacement of these high-affinity probes can differentiate molecules with higher affinity for the ribosome. On the other hand, Probe 203 with its faster kinetics and slightly lower affinity has the greatest potential for HTS by minimizing the time required for assays and allowing the use of higher levels of fluorophore (greater fluorescence signal) while maintaining a concentration below the K_{d} that is desirable for FP HTS.

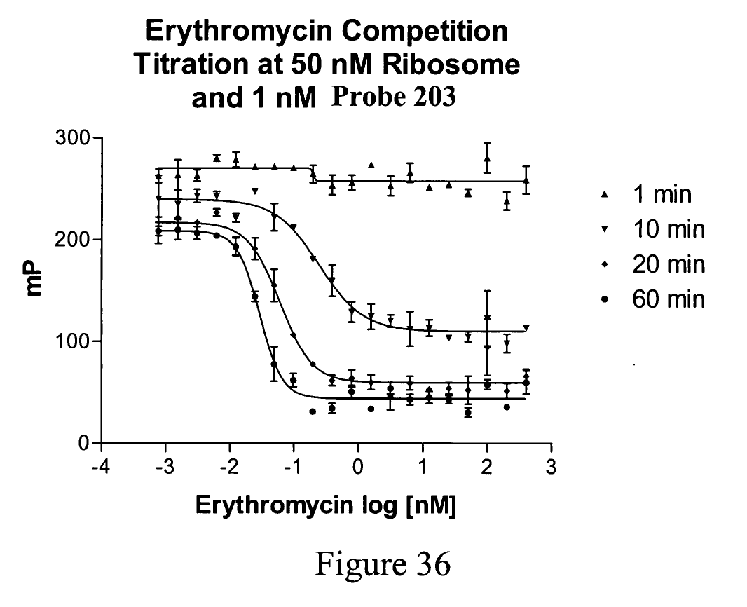

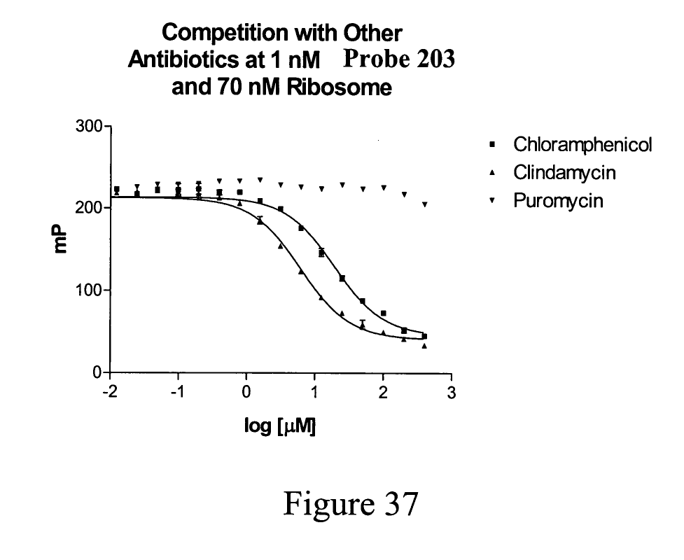

Competition with Fluorescently Labeled Probe: To show that the probes were binding to the 70S ribosome in a biologically relevant manner we demonstrated the ability to compete off the probe with the parent compound, as well as with other antibiotics that are known to bind in the same area. The competition experiments were carried out in the same buffer as the binding experiments, at a probe concentration that maximized FP signal and a ribosome concentration 150-200% above the determined K_{d}. The compounds of interest were titrated from a range of 400 μM to 1.5 nM and readings were taken at various time points after adding compound to probe-bound ribosomes (see FIG. 36 and FIG. 37). Both unlabeled erythromycin and other ribosomal binding antibiotics which should show competition were able to compete out Probe 203 at expected IC₅₀ levels (Erythromycin at 30 nM, Chloramphenicol at 19 μM, and Clindamycin at 6.2 μM). These calculate to reasonable affinities of 7.1 μM for chloramphenicol and 2.3 μM for clindamycin, but erythromycin affinity cannot be determined with this probe because of its higher affinity. This again points to the utility of high affinity probes like Exampe 8 and Example 9 for resolving the affinity of tight-binding competitors. Antibiotics that bind to more distant regions of the ribosome, such as puromycin, did not show competition. These data demonstrate that the fluorescent probes are binding to ribosomes in the same manner as the parent antibiotics. In addition, the results prove the utility of these macrolide probes for detecting displacement by competitive binders, for which many uses have already been detailed above.

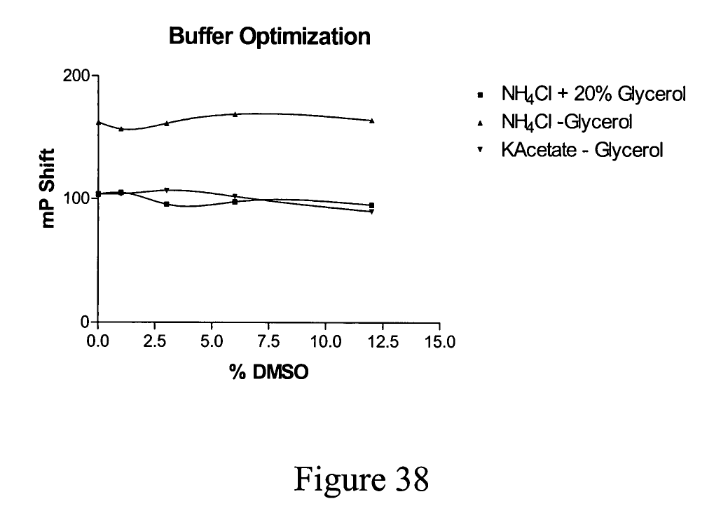

Transition to High Throughput Screening: We created a system for high density screening of novel antibiotic probes and ribosome sites with increased maximum signal resolution compared to previously reported procedures (Turconi, S. et al. J. Biomolecular Screening, 2001, 6, 275-290). Furthermore, we determined screening conditions that allowed screening at much higher compound concentration to detect weaker inhibitors of ribosome function as starting points for drug development. The buffer was optimized for maximum mP signal increase of bound vs. unbound ligand as well as consistency of reads. We found that 0.05% Tween is necessary for reduction of meniscus effects which affects repeatability of multiple reads. Glycerol was found to significantly decrease total mP shift without providing any clear benefit to the assay. We eliminated glycerol altogether from our assay, in sharp contrast to the substantial 20% glycerol content in reported procedures (Turconi, S. et al. J. Biomolecular Screening, 2001, 6, 275-290). Binding of probe was relatively insensitive to the concentration of Mg so long as this was between 2.5 and 40 mM. Additional salt types and concentrations were looked at and 100 mM NH₄Cl was found to be optimal. We looked at a wide range of both KOAc and NH₄Cl and found that KOAc had a clear decrease in signal (see FIG. 38).

According to reported procedures (Turconi, S. et al. J. Biomolecular Screening, 2001, 6, 275-290), screening was done at 10 μM concentration of compounds (allowing detection of binders only of affinity better than 4 μM) and 1% DMSO. We examined the affects of DMSO on our HTS competition in an effort to find a significantly higher level of DMSO that would be tolerated by the assay and yet maintain greater solubility of compounds when screened at concentrations as high as 50 μM (allowing detection of binders with affinity as high as 18 μM) We initially saw strong DMSO effects suggesting increased DMSO was contributing to decreased signal, but we found that the effects resulted from autofluorescence of the DMSO itself leading to a lower mP shift. By always running blank corrections at the appropriate DMSO concentration this shift can be eliminated. Using a background correction on the reader specific to each DMSO concentration, we found that the mP signal did not show a significant loss up to 10% DMSO (see FIG. 38). We ran the final assay at 6% DMSO to balance keeping the 70S ribosome in an as biologically relevant a state as possible with higher solubility of library compounds. The final conditions used in the assay were: 20 mM MgCl₂, 100 mM NH₄Cl, 30 mM Tris-HCl, pH 7.5, 0.05% Tween-20, and 6% DMSO.

Automation: The high-throughput screen was performed on a single pod, Beckman Biomek FX with a 384 head. A Beckman Positive Position ALP (“Automated Labware Positioner”) was added to the robot to assist in accurately positioning 1536-well plates so that pipetting could be performed in the 4 quadrants of the plate with the 384 head. A 1536-well format was selected to increase throughput while decreasing reagent cost. Specifically, over 10,000 compounds could be screened in less than 1.5 hours utilizing the 1536-well format with a volume of only 8.5 μL per well.

The ribosome and probe solution was premixed and placed in a V&P Scientific 384-well, dimpled bottom reagent reservoir with control wells. The control wells included no probe blanks, DMSO only with ribosome/probe (negative control), an eight concentration titration of clindamycin from 200 μM (positive control) down to 91 nM, and probe wells lacking ribosome (backup positive control). Displacement by clindamycin as a positive control was found to give more reproducible results and is in principle more appealing than no ribosome controls as used for HTS by others (Turconi, S. et al. J. Biomolecular Screening, 2001, 6, 275-290). Initially, 7.5 μL of ribosome and probe mix, along with the controls, were added to the 1536-well plates. A special pipetting procedure involving slow dispensing while following the liquid level was developed in order to minimize bubble formation in the wells and reduce false hits. Additionally, the FX was calibrated to accurately dispense low volumes following the Beckman technical bulletin T-1915A, “Improving Accuracy by Use of Technique Calibration”.

After washing the tips with water and 100% DMSO, a 45% or 36% DMSO solution was added to four intermediate 384-well compound plates. The percent of DMSO depended on the concentration of the compound plate (5 mM or 2 mM respectively). For 5 mM compound plates, 1 μL of compound was added to an intermediate plate, mixed, and then 1 μL added to one quadrant of the 1536-well plate. For 2 mM compound plates, 2.6 μL of compound was added to the intermediate plate, mixed, and 1 μL of this solution was added to the 1536-well plate. The final volume in each 1536-well plate was 8.5 μL with a final DMSO concentration of approximately 6% and a compound concentration of 50 μM.

After the assay was completed, plates were incubated for a minimum of 4 hours and then read on a Perkin-Elmer Envision plate reader. The Envision is capable of reading fluorescence, absorption, luminescence, and fluorescence polarization. The optical module selected for reading the FP signal was the Optimized FITC FP Dual Emission Label (part #2100-8060-Fl) which provided an excitation wavelength of 480 nm and emission wavelength of 535 nm for both s and p polarizations. The plates were read using 30 flashes per well which resulted in a read time of approximately 4 minutes per 1536-well plate.

One skilled in the art readily appreciates that the disclosed invention is well adapted to carry out the mentioned and inherent objectives. Linkers, fluorophores, ligands of bacterial ribosome and functional equivalents thereof, pharmaceutical compositions, treatments, methods, procedures and techniques described herein are presented as representative of the preferred embodiments and are not intended as limitations of the scope of the invention. Thus, other uses will occur to those skilled in the art that are encompassed within the spirit and scope of the described invention.

1. A fluorescent probe comprising:
a bacterial ribosome ligand; and
a fluorophore coupled to the bacterial ribosome ligand;
wherein, the bacterial ribosome ligand comprises an antibiotic; and the fluorophore comprises a molecule that emits fluorescent light following excitation.

2. The fluorescent probe of claim 1, further comprising a linker that couples the bacterial ribosome ligand and the fluorophore, the linker comprising a carbon chain having 0 to 16 carbons.

3. The fluorescent probe of claim 2, wherein the carbon chain is interrupted by 1 to 6 heteroatoms, functional groups, carbocycles and heterocycles, or by 1 to 6 substituents.

4. The fluorescent probe of claim 1, wherein the antibiotic comprises a 14-membered ring macrolide, a 15-membered ring macrolide, a 16-membered ring macrolide, a tetracycline, an aminoglycoside, an oxazolidinone, clindamycin, puromycin, chloramphenicol, spectinomycin, streptomycin, amikacin, or a pleuromutilin.

5. The fluorescent probe of claim 1, wherein the fluorophore comprises BODIPY FL, BODIPY FL-X, BODIPY FL C5, BODIPY TMR, Cy3B, fluorescein, rhodamine red, or dipyrrinone.

6. The fluorescent probe of claim 1, wherein the antibiotic is a 14-, 15- or 16-membered ring macrolide; the fluorophore selected from a BODIPY, BODIPY FL, BODIPY TMR, or Cy3B; and the antibiotic and fluorophore are coupled together by a linker comprising a carbon chain having 0 to 16 carbons.

7. The fluorescent probe of claim 6, wherein the carbon chain is interrupted by 1 to 6 heteroatoms, functional groups, carbocycles and heterocycles, or 1 to 6 substituents.

8. A fluorescent probe comprising: 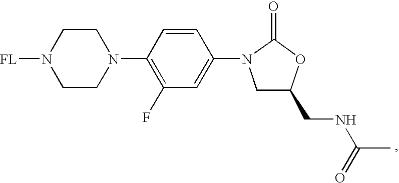
wherein,
FL is the fluorophore comprising:

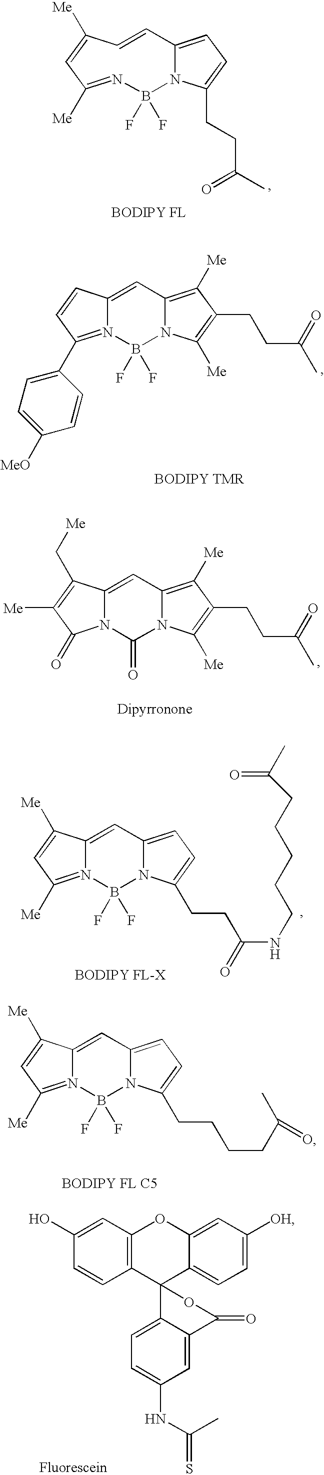

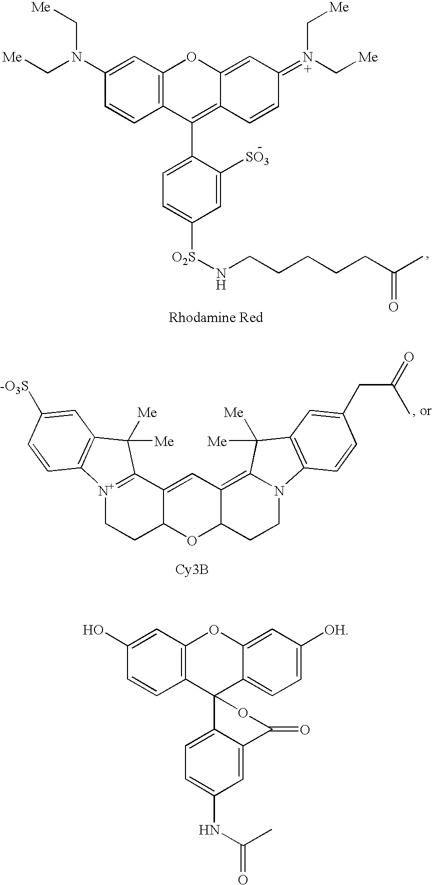

9. A fluorescent probe comprising: 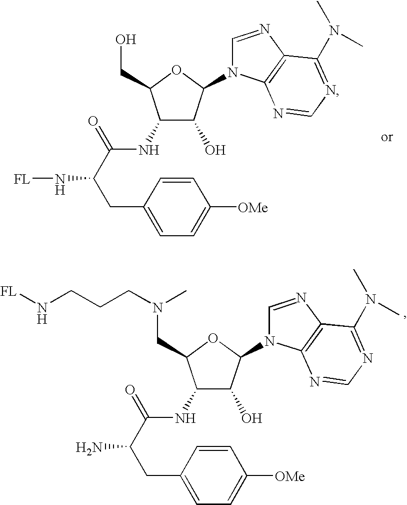
wherein,
FL is the fluorophore comprising:

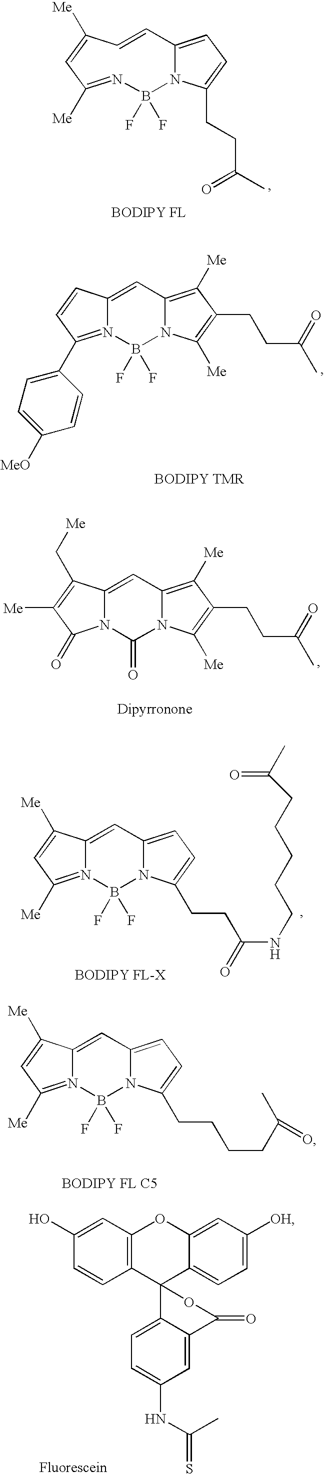

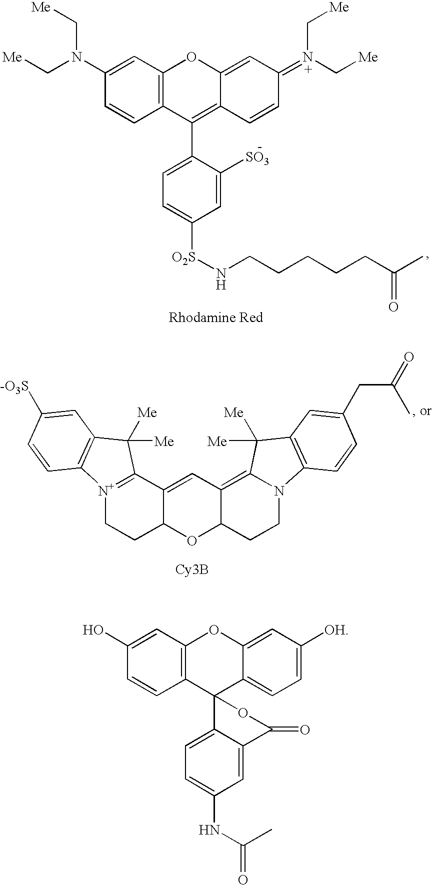

10. A fluorescent probe comprising: 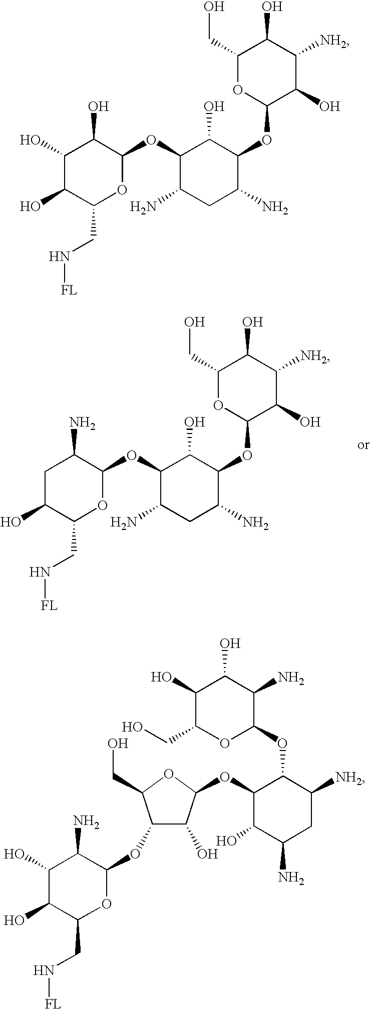
wherein,
FL is the fluorophore comprising:

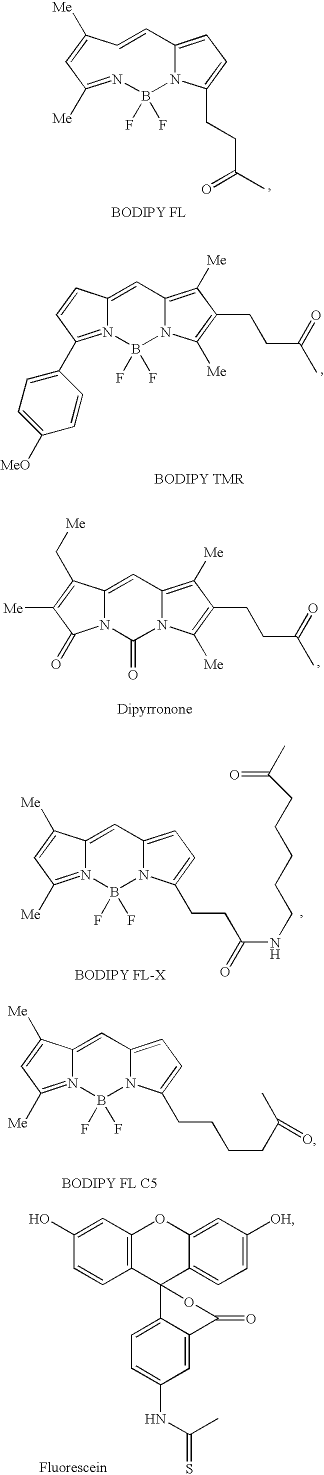

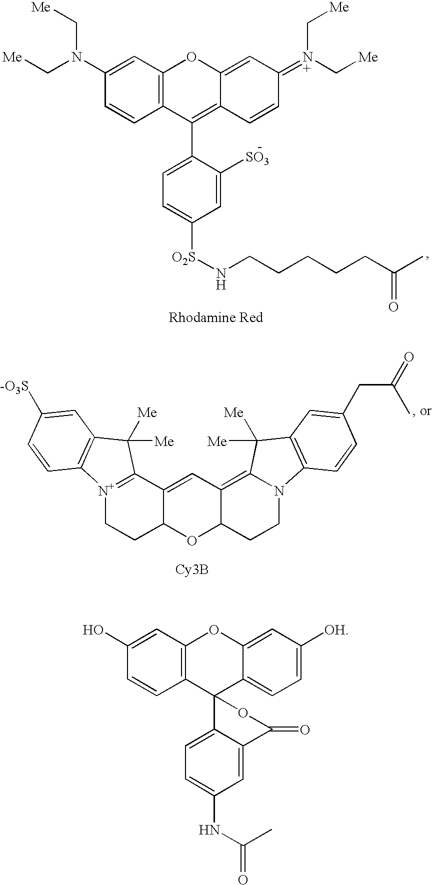

11. A fluorescent probe comprising: 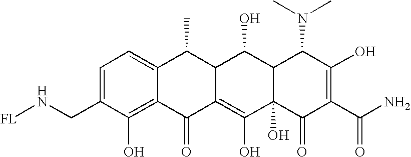
wherein,
FL is the fluorophore comprising:

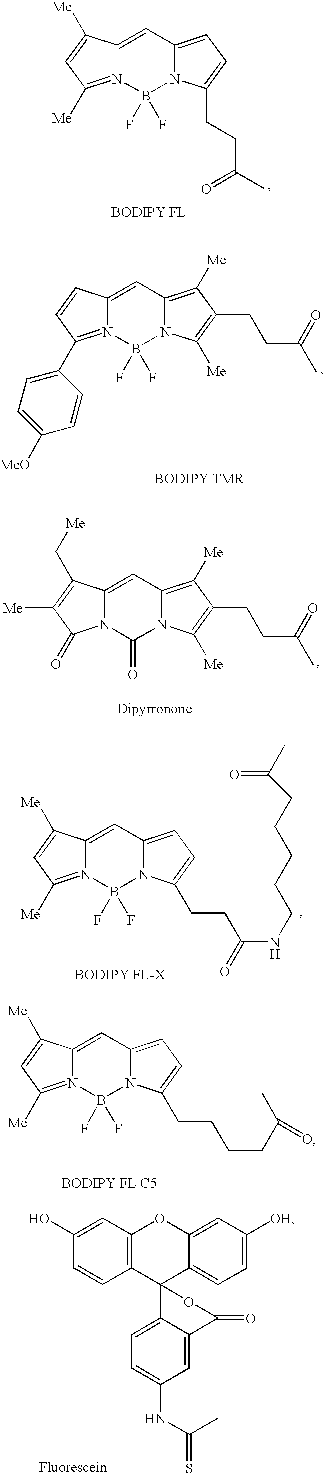

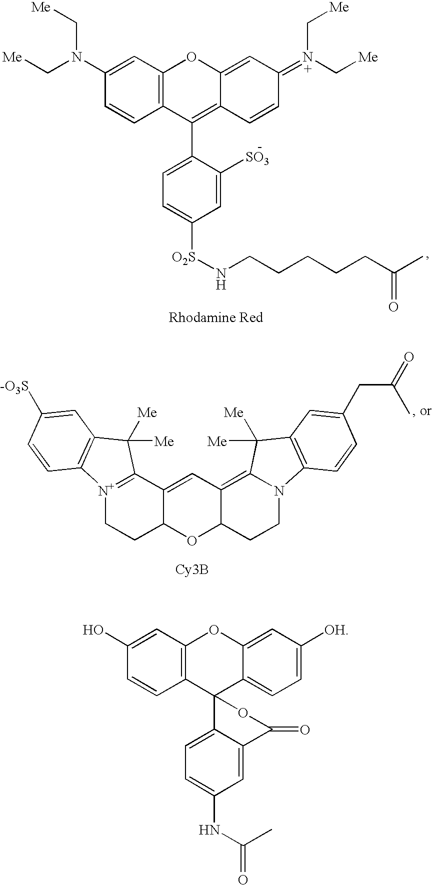

12. A fluorescent probe comprising: 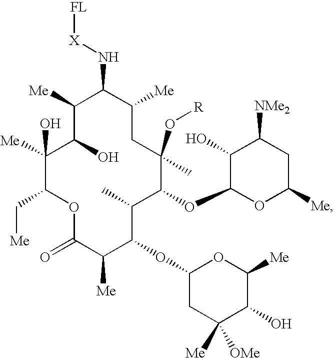
wherein,
FL is the fluorophore comprising: 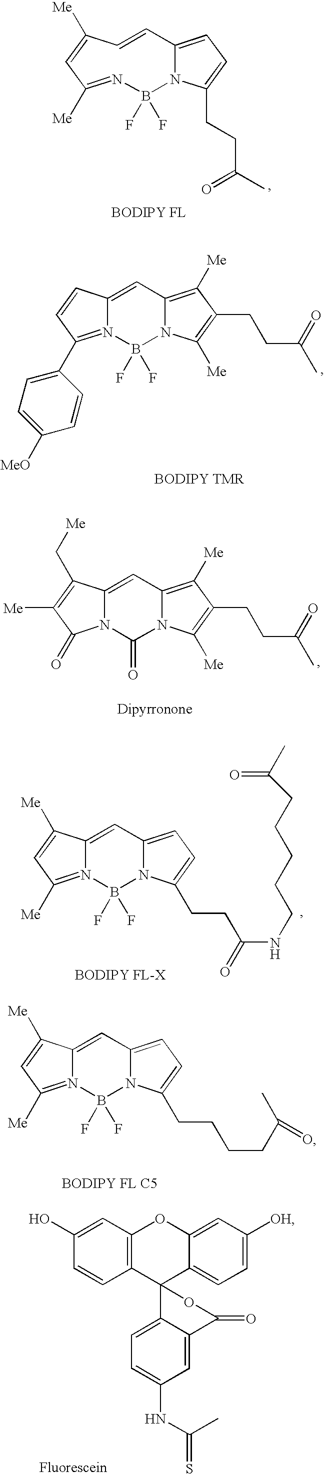 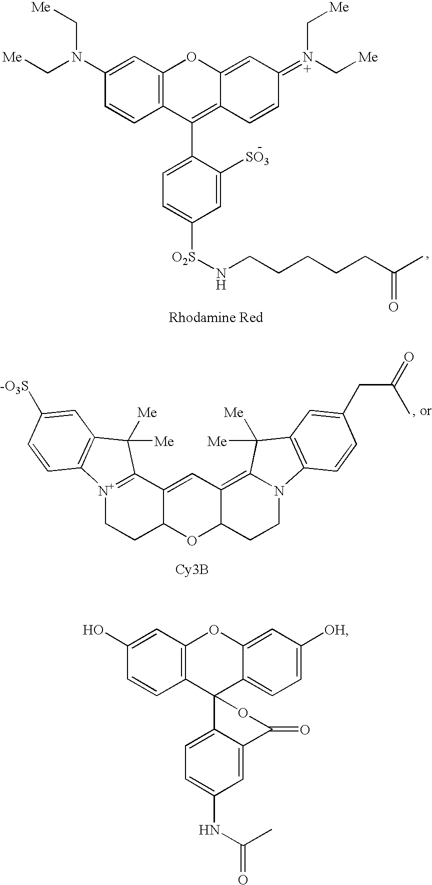
X is none, —(CH₂)ₙNH—, —C(O)—(CH₂)ₙ—NH,— or —C(O)—NH—(CH₂)ₙ—NH—, wherein n is a number between 2 and 6; and
R is H or a low alkyl group.

13. A fluorescent probe comprising: 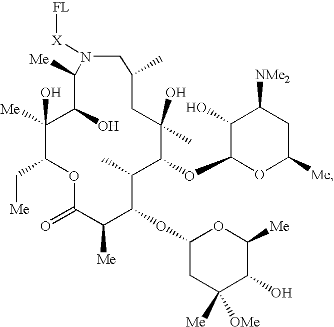
wherein,
FL is the fluorophore comprising: 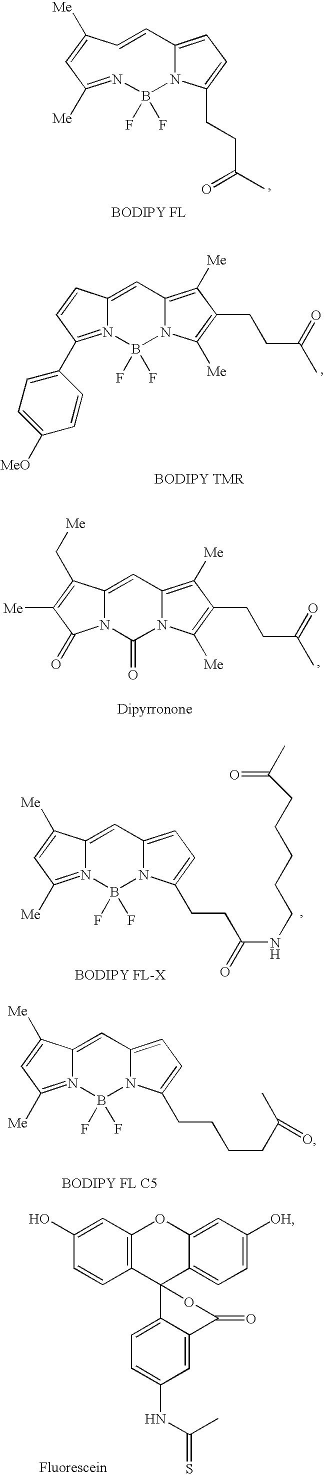 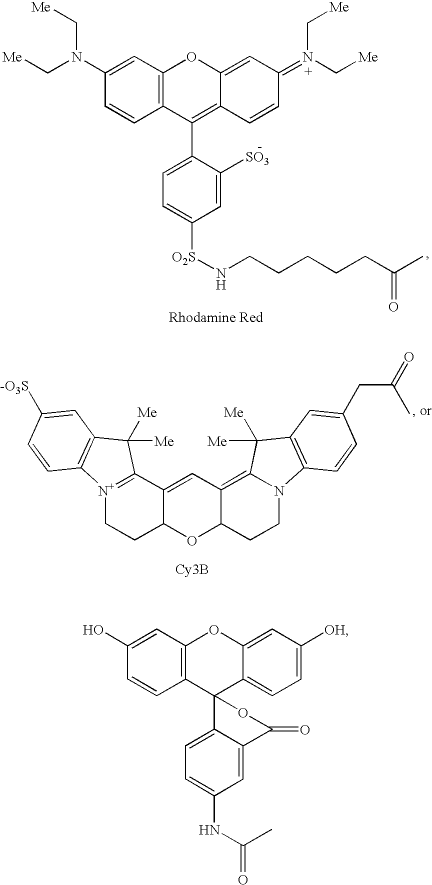
X is none; —(CH₂)ₙNH—; —C(O)—(CH₂)ₙ—NH—; or —C(O)—NH—(CH₂)ₙ—NH—, wherein n is a number between 2 and 6.

14. A fluorescent probe comprising: 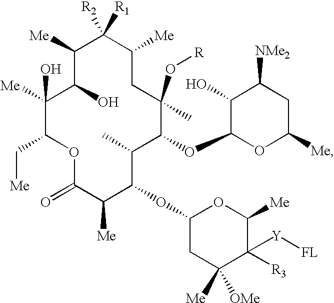
wherein,
FL is the fluorophore comprising: 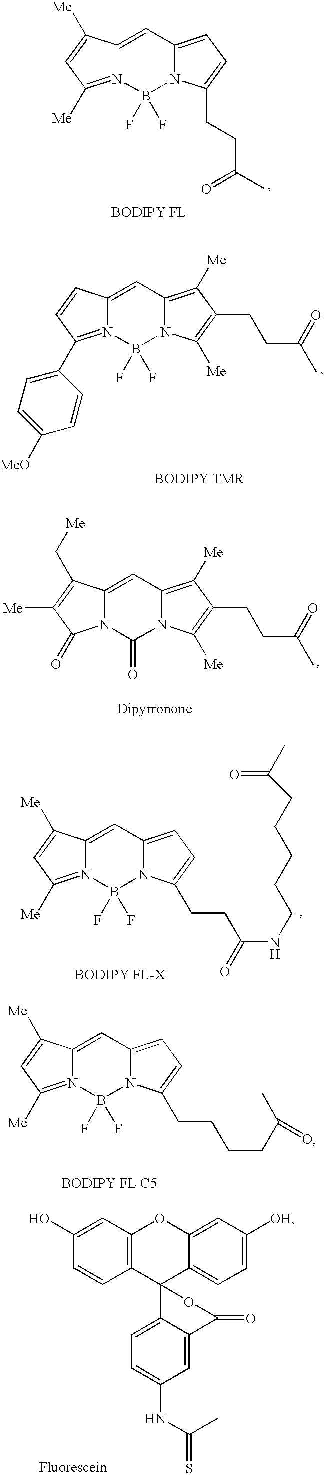 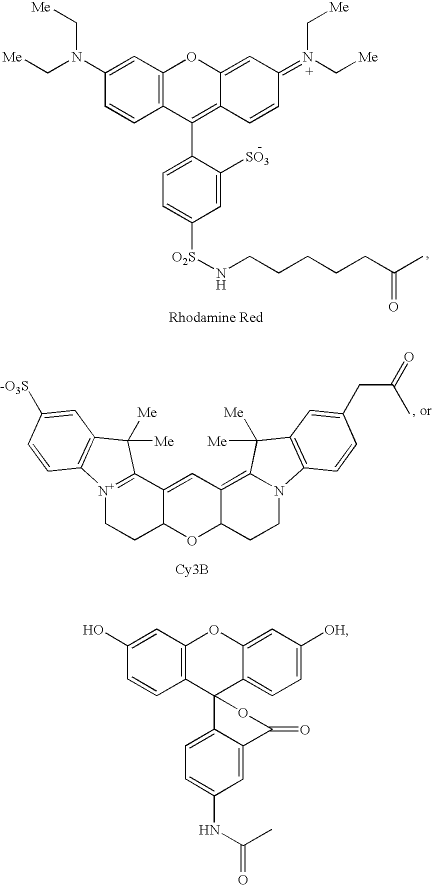
Y comprises: —NH—(CH₂)ₙ—NH—; —NH—C(O)—(CH₂)ₙ—NH—; —NH—C(O)—NH—(CH₂)ₙ—NH—; —O—C(O)—NH—(CH₂)ₙ—NH—; —CH₂—NH—(CH₂)ₙ—NH—; CH₂—NH—C(O)—(CH₂)ₙ—NH—; or —CH₂—NH—C(O)—NH—(CH₂)ₙ—NH—, wherein n is a number between 2 and 6;
R is H or C₁-C₆ alkyl;
one of R₁ or R₂ is H and the other is selected from: —NR^{a}R^{b}, —OH, or R₁ and R₂ together to form ═O; R^{a} and R^{b} are independently selected from groups consisting of: C₁-C₆ alkyl, —C(O)R^{c}, —C(O)OR^{C}, —C(O)NR^{d}R^{e}, or R^{a} and R^{b} together to form a 3-8 membered heterocycle ring with 1-3 heteroatoms in the ring, optionally substituted with 1-3 substituents; R^{c} is selected from C₁-C₆ alkyl, substituted C₁-C₆ alkyl, aryl, substituted aryl, heteroaryl, and substituted heteroaryl; R^{d} and R^{e} are C₁-C₆ alkyl, aryl, heteroaryl, substituted heteroaryl, or R^{d} and R^{e} together to form a 3-8 membered heterocycle ring; and
R₃ comprises —H or —OH.

15. A fluorescent probe comprising: 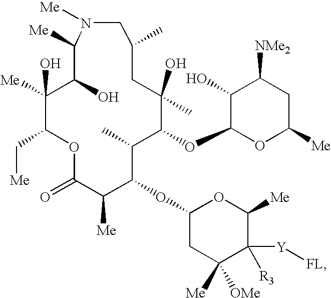
wherein,
FL is the fluorophore comprising: 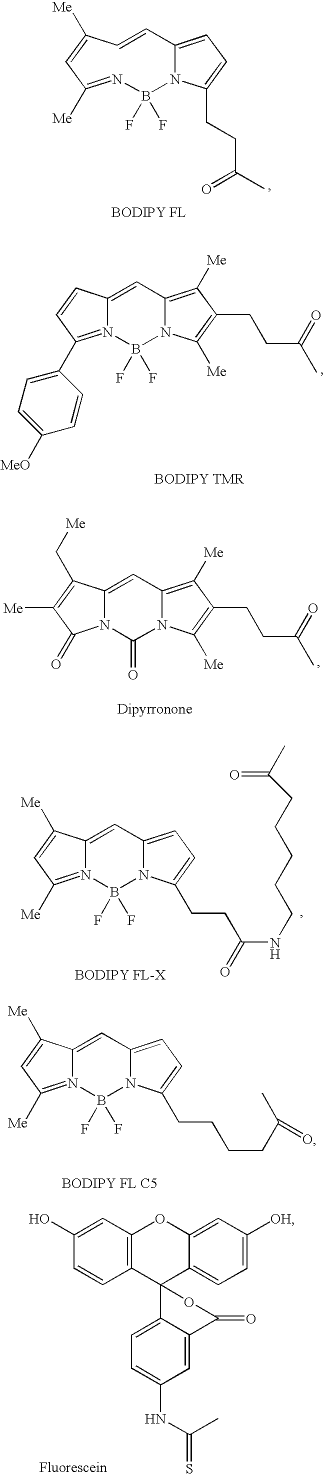 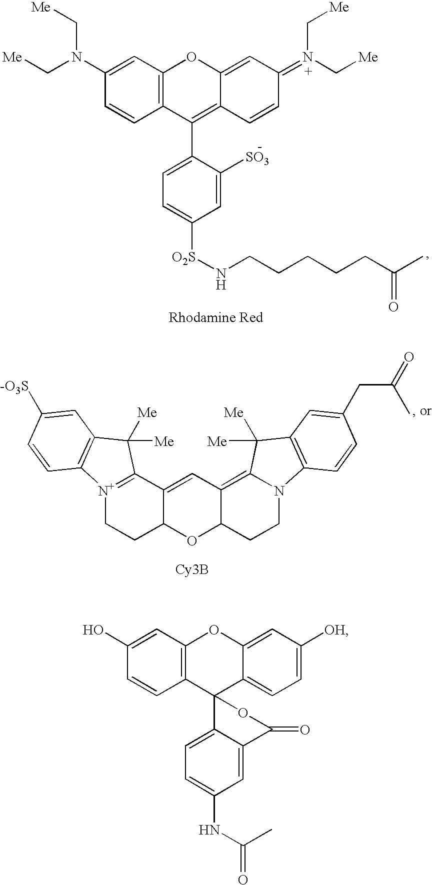
Y comprises: —NH—(CH₂)ₙ—NH—; —NH—C(O)—(CH₂)ₙ—NH—; —NH—C(O)—NH—(CH₂)ₙ—NH—; —O—C(O)—NH—(CH₂)ₙ—NH—; —CH₂—NH—(CH₂)ₙ—NH—; —CH₂—NH—C(O)—(CH₂)ₙ—NH—; or CH₂—NH—C(O)—NH—(CH₂)ₙ—NH—, wherein n is a number between 2 and 6; and
R₃ comprises —H or —OH.

16. A fluorescent probe comprising: 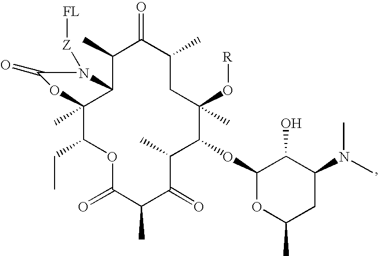
wherein,
FL is the fluorophore comprising: 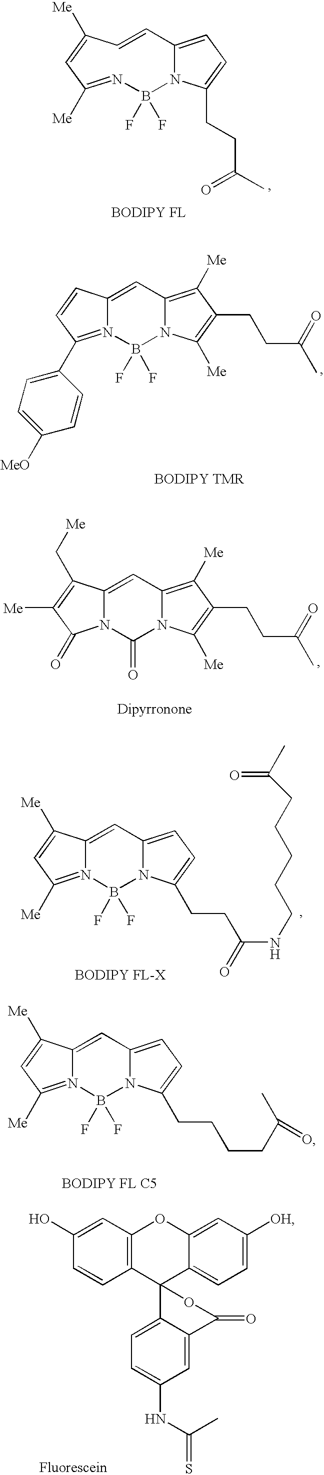 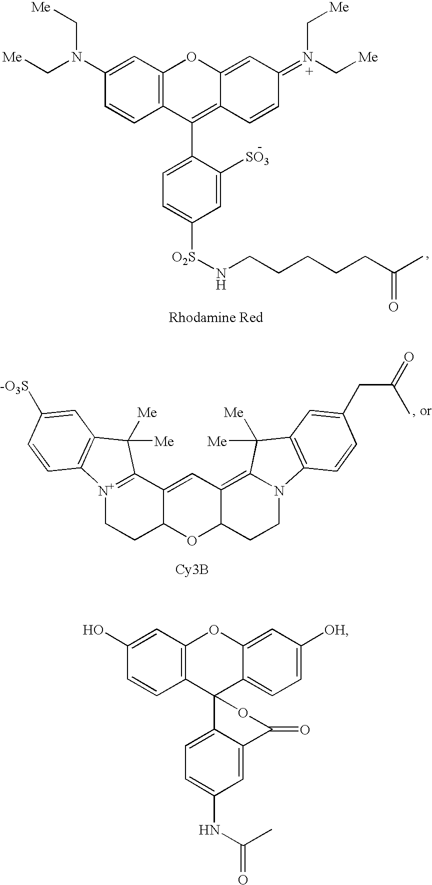
R is H or a low alkyl group,
Z is -A-(CH₂)ₙ—NH—, wherein n is a number between 2 and 6, A is absence, —NH—, or —O—.

17. A fluorescent probe comprising: 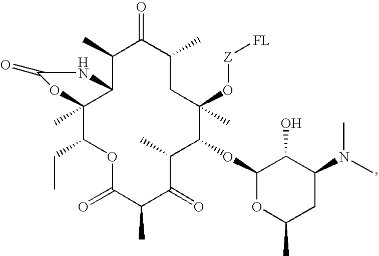
wherein,
FL is the fluorophore comprising: 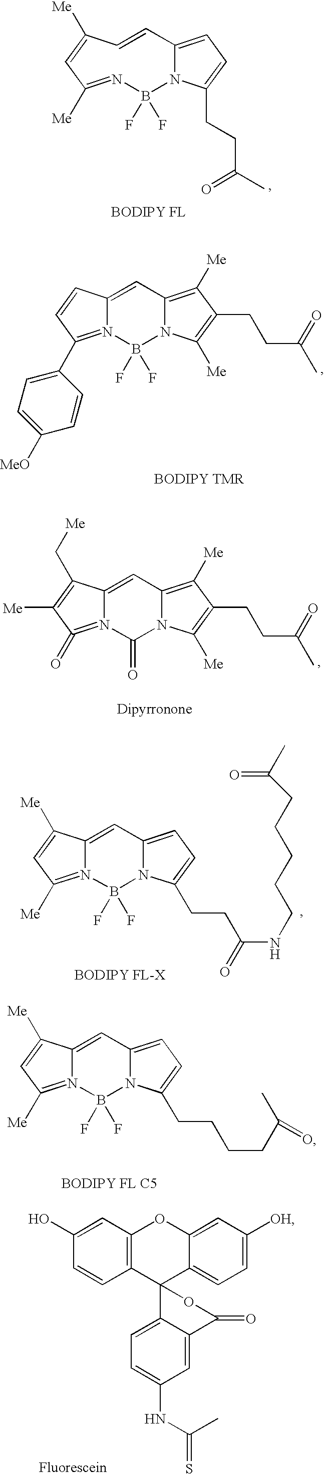 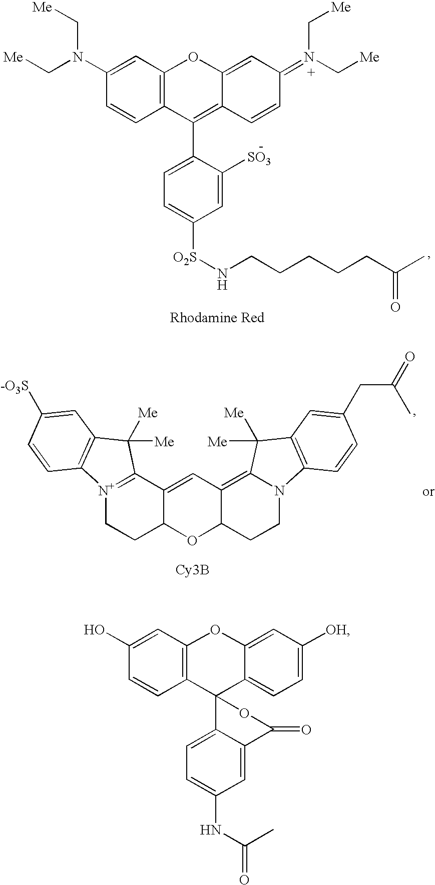
Z is —(CH₂)ₙNH—, wherein n is a number between 2 and 6.

18. A fluorescent probe comprising: 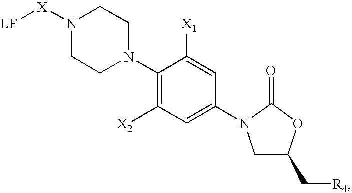
wherein,
FL is the fluorophore comprising: 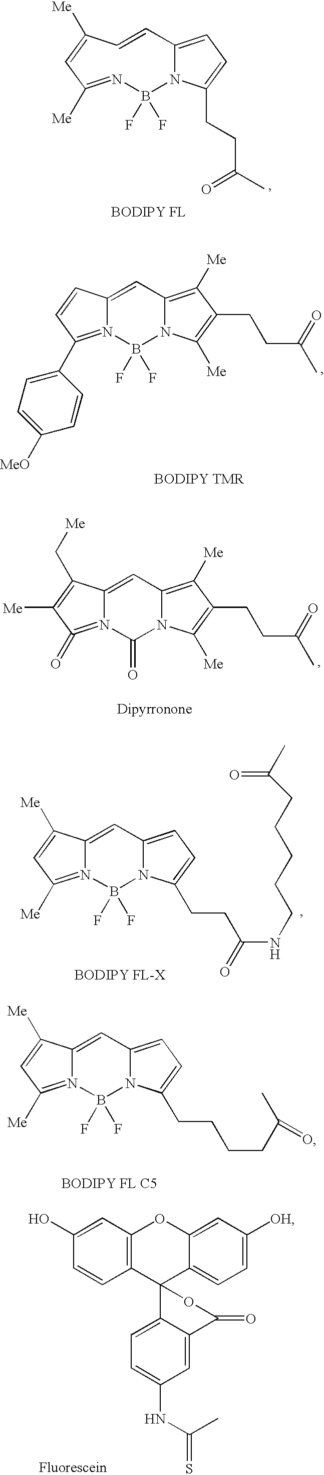 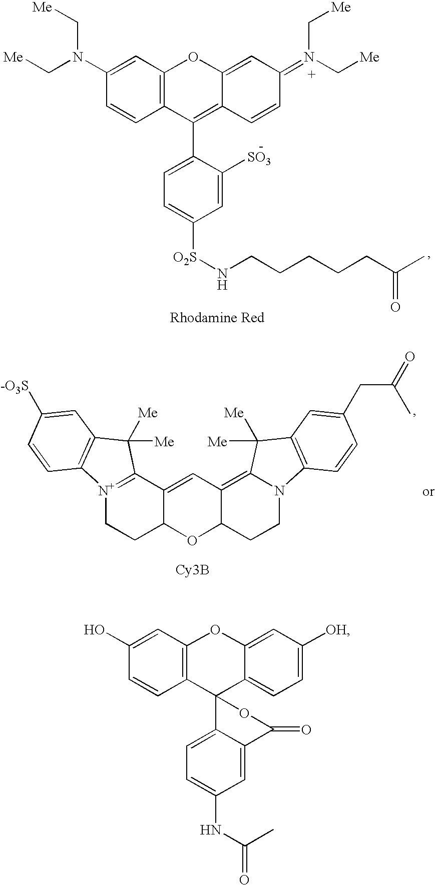
X is none; —(CH₂)ₙNH—; —C(O)—(CH₂)ₙ—NH—; or —C(O)—NH—(CH₂)ₙ—NH—, wherein n is a number between 2 and 6;
X₁ and X₂ are independently —H or —F; and
R₄ comprises:

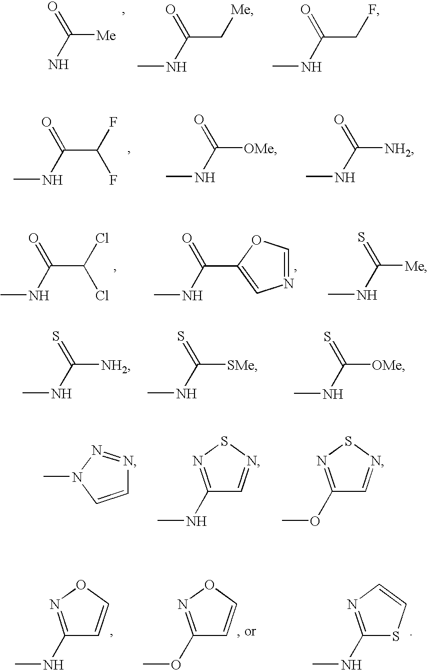

19. A fluorescent probe comprising: 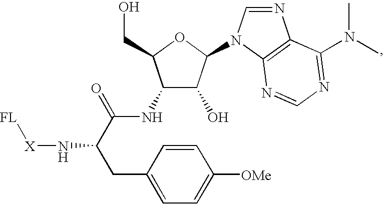
wherein,
FL is the fluorophore comprising: 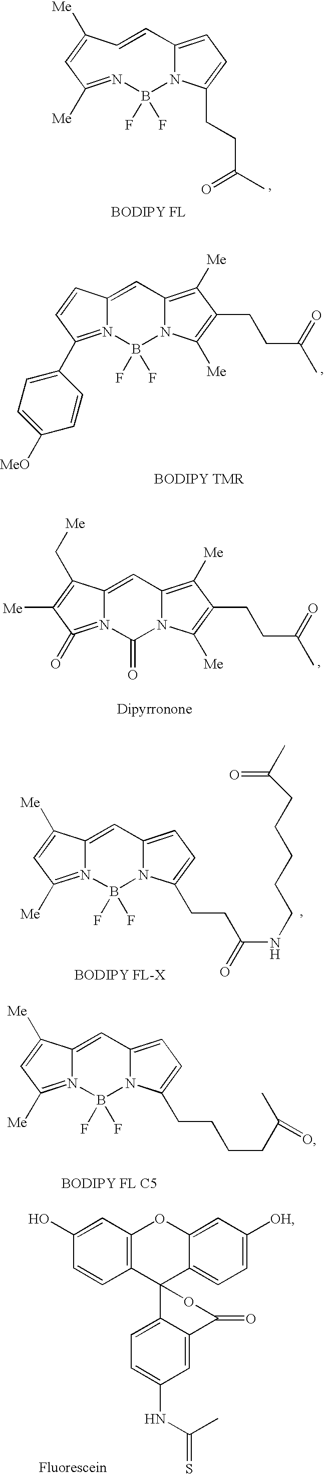 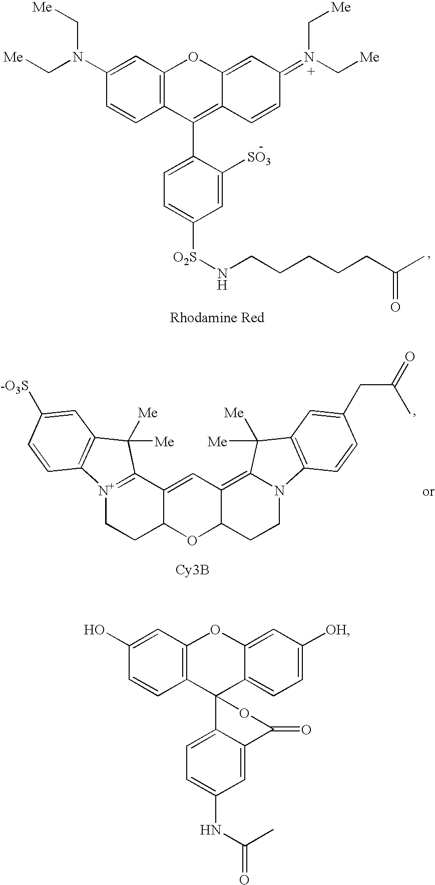
X is none; —(CH₂)ₙNH—; —C(O)—(CH₂)ₙ—NH—; or —C(O)—NH—(CH₂)ₙ—NH—, wherein n is a number between 2 and 6.

20. A fluorescent probe comprising: 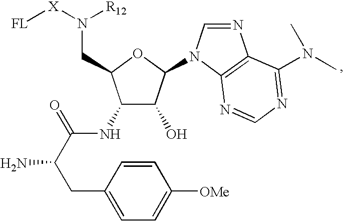
wherein,
FL is the fluorophore comprising: 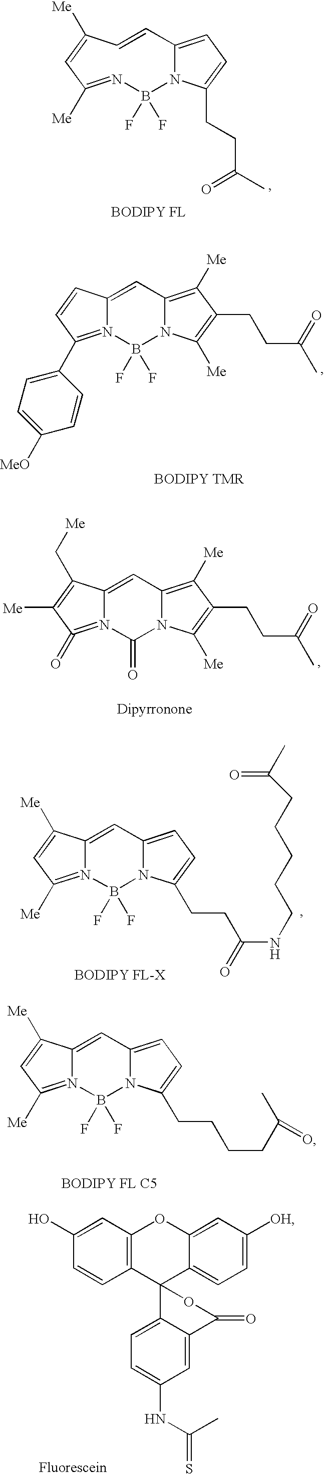 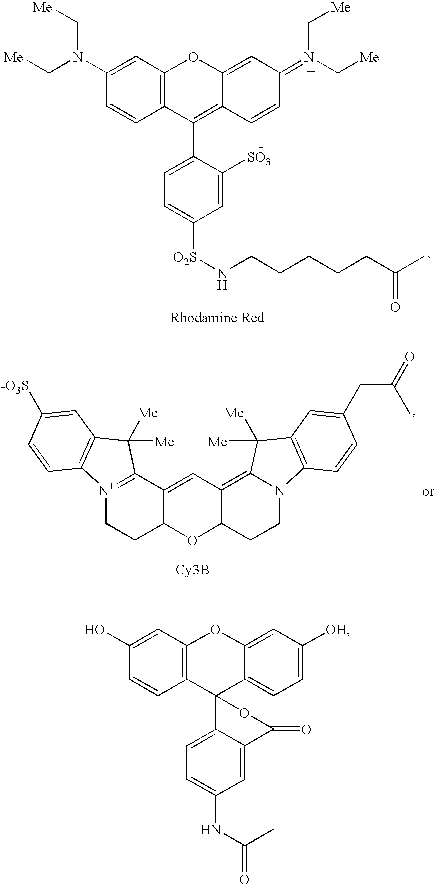
X is none; —(CH₂)ₙNH—; —C(O)—(CH₂)ₙ—NH—; or —C(O)—NH—(CH₂)ₙ—NH—, wherein n is a number between 2 and 6; and
R₁₂ is H or low alkyl.

21. A fluorescent probe comprising: 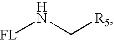
wherein
FL is the fluorophore comprising: 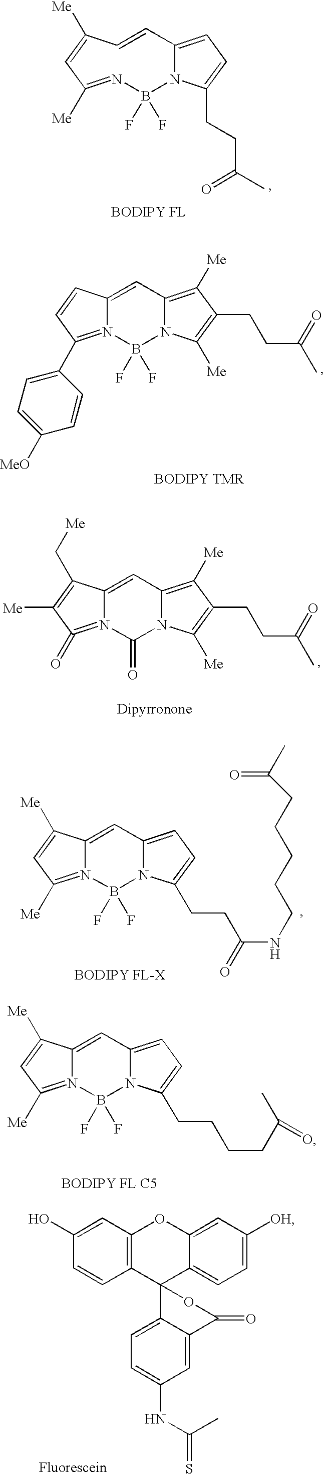 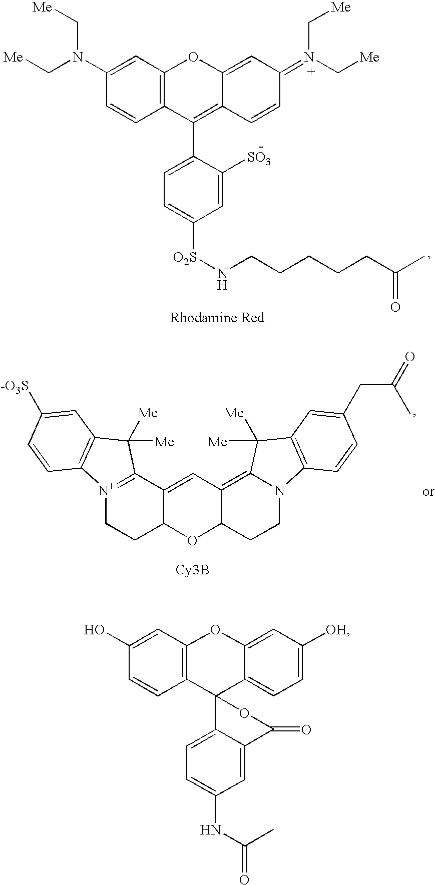
R₅ comprises:

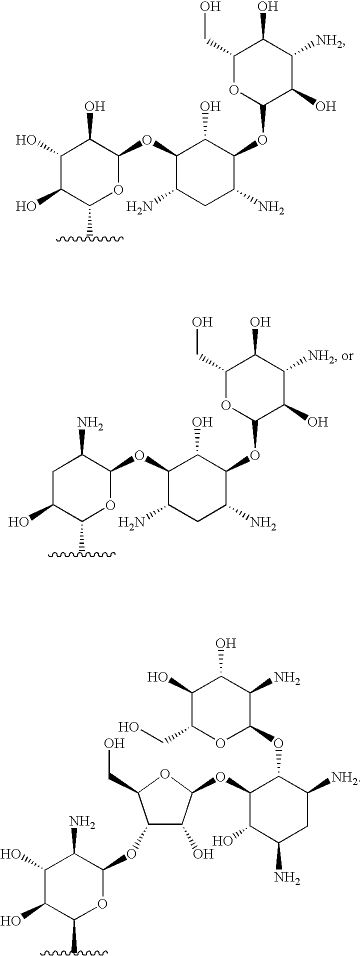

22. A fluorescent probe comprising:

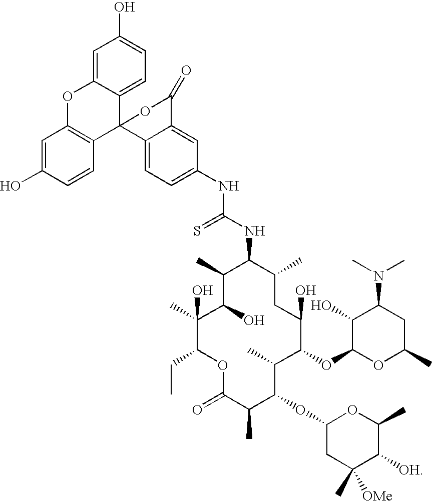

23. A fluorescent probe comprising:

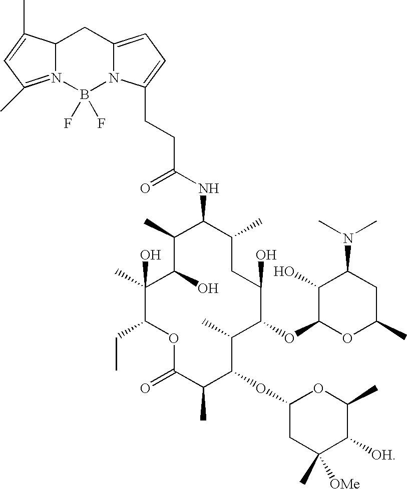

24. A fluorescent probe comprising:

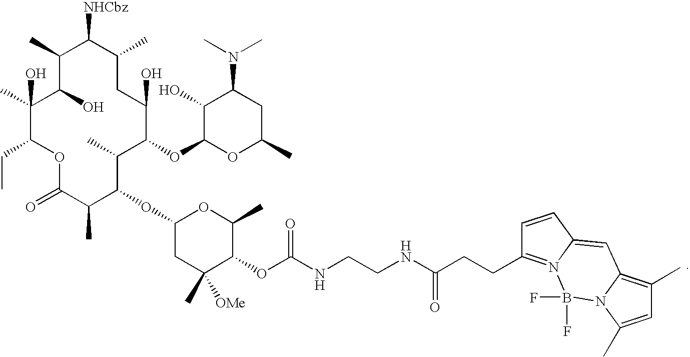

25. A fluorescent probe comprising:

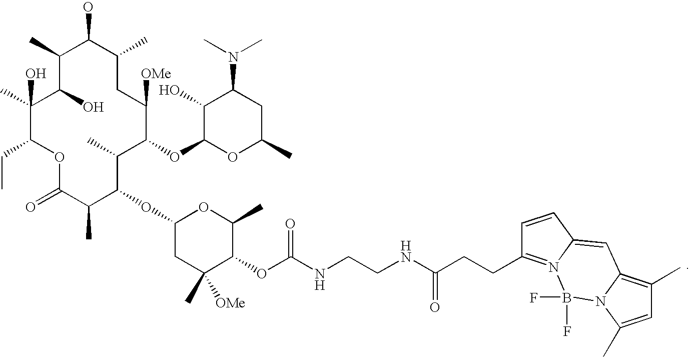

26. A method for identifying and characterizing a ribosome ligand comprising:
contacting a bacterial ribosome with a fluorescent probe for a first period of time forming a probe-ribosome complex;
exposing the probe-ribosome complex to a test compound for a second period of time forming a compound-probe-ribosome mixture;
passing the compound-probe-ribosome mixture through an examination zone;
collecting data on a fluorescence emission intensity and fluorescence polarization of the compound-probe-ribosome mixture and determining if the test compound is a ribosome ligand by disrupting the probe-ribosome complex,
wherein,
the fluorescent probe comprises an antibiotic coupled to a fluorophore and the fluorophore comprises a molecule that emits fluorescent light following excitation;
the first period of time is greater than 1 minute; and
the second period of time is greater than 1 minute.

27. The method of claim 26, wherein the bacterial ribosome is from E. coli or S. aureus.

28. The method of claim 26, wherein the ribosomes used for screening are derived or purified from Streptococcus pneumoniae, Streptococcus pyogenes, Enterococcus fecalis, Enterococcus faecium, Klebsiella pneumoniae, Enterobacter sps., Proteus sps., Pseudomonas aeruginosa, E. coli, Serratia marcesens, S. aureus, Coag. Neg. Staph., Acinetobacter sps., Salmonella sps, Shigella sps., Helicobacter pylori, Mycobacterium tuberculosis, Mycobacterium avium, Mycobacterium intracellulare, Mycobacterium fortuitum, Mycobacterium chelonae, Mycobacterium kansasit, Haemophilus influenzae, Stenotrophomonas maltophilia, or Streptococcus agalactiae.

29. The method of claim 26, wherein the ribosomes used for screening are derived or purified from Acinetobacter calcoaceticus, A. haemolyticus, Aeromonas hydrophilia, Bacteroides fragilis, B. distasonis, Bacteroides 3452A homology group, B. vulgatus, B. ovalus, B. thetaiotaomicron, B. uniformis, B. eggerthii, B. splanchnicus, Branhamella catarrhalis, Campylobacterfetus, C. jejuni, C. coli, Citrobacterfreundii, Clostridium difficile, C. diphtheriae, C. ulcerans, C. accolens, C. afermentans, C. amycolatum, C. argentorense, C. auris, C. bovis, C. confusum, C. coyleae, C. durum, C. falsenit, C. glucuronolyticum, C. imitans, C. jeikeium, C. kutscheri, C. kroppenstedtii, C. lipophilum, C. macginleyi, C. matruchoti, C. mucifaciens, C. pilosum, C. propinquum, C. renale, C. riegelii, C. sanguinis, C. singulare, C. striatum, C. sundsvallense, C. thomssenit, C. urealyticum, C. xerosis, Enterobacter cloacae, E. aerogenes, Enterococcus avium, E. casseliflavus, E. cecorum, E. dispar, E. durans, E. faecalis, E. faecium, E. flavescens, E. gallinarum, E. hirae, E. malodoratus, E. mundtii, E. pseudoavium, E. raffinosus, E. solitarius, Francisella tularensis, Gardnerella vaginalis, Helicobacter pylori, Kingella dentrificans, K. kingae, K. oralis, Klebsiella pneumoniae, K. oxytoca, Moraxella catarrhalis, M. atlantae, M. lacunata, M. nonliquefaciens, M. osloensis, M. phenylpyruvica, Morganella morganii, Parachlamydia acanthamoebae, Pasteurella multocida, P. haemolytica, Proteus mirabilis, Proteus vulgaris, Providencia alcalifaciens, P. rettgeri, P. stuartit, Serratia marcescens, Simkania negevensis, Streptococcus pneumoniae, S. agalactiae, S. pyogenes, Treponema pallidum, Vibrio cholerae, or V. parahaemolyticus.

30. The method of claim 26, wherein the ribosomes used for screening are derived or purified from facultative intracellular bacteria comprising: Bordetella pertussis, B. parapertussis, B. bronchiseptica, Burkholderia cepacia, Escherichia coli, Haemophilus actinomycetemcomitans, H. aegyptius, H. aphrophilus, H. ducreyi, H. felis, H. haemoglobinophilus, H. haemolyticus, H. influenzae, H. paragallinarum, H. parahaemolyticus, H. parainfluenzae, H. paraphrohaemolyticus, H. paraphrophilus, H. parasuis, H. piscium, H. segnis, H. somnus, H. vaginalis, Legionella adelaidensis, L. anisa, L. beliardensis, L. birminghamensis, L. bozemanii, L. brunensis, L. cherrii, L. cincinnatiensis, Legionella drozanskii L. dumoffli, L. erythra, L. fairfieldensis, L. fallonii, L. feeleii, L. geestiana, L. gormanii, L. gratiana, L. gresilensis, L. hackeliae, L. israelensis, L. jordanis, L. lansingensis, Legionella londiniensis L. longbeachae, Legionella lytica L. maceachernii, L. micdadei, L. moravica, L. nautarum, L. oakridgensis, L. parisiensis, L. pittsburghensis, L. pneumophila, L. quateirensis, L. quinlivanii, L. rowbothamii, L. rubrilucens, L. sainthelensi, L. santicrucis, L. shakespearei, L. spiritensis, L. steigerwaltii, L. taurinensis, L. tucsonensis, L. wadsworthii, L. waltersii, L. worsleiensis, Listeria denitrificans, L. grayi, L. innocua, L. ivanovii, L. monocytogenes, L. seeligeri, L. welshimeri, Mycobacterium abscessus, M. africanum, M. agri, M. aichiense, M. alvei, M. asiaticum, M. aurum, M. austroafricanum, M. avium, M. bohemicum, M. bovis, M. branderi, M. brumae, M. celatum, M. chelonae, M. chitae, M. chlorophenolicum, M. chubuense, M. confluentis, M. conspicuum, M. cookii, M. diernhoferi, M. doricum, M. duvalii, M. elephantis, M. fallax, M. farcinogenes, M. flavescens, M. fortuitum, M. frederiksbergense, M. gadium, M. gastri, M. genavense, M. gilvum, M. goodii, M. gordonae, M. haemophilum, M. hassiacum, M. heckeshornense, M. heidelbergense, M. hiberniae, M. immunogenum, M. intracellulare, M. interjectum, M. intermedium, M. kansasii, M. komossense, M. kubicae, M. lentiflavum, M. leprae, M. lepraemurium, M. luteum, M. madagascariense, M. mageritense, M. malmoense, M. marinum, M. microti, M. moriokaense, M. mucogenicum, M. murale, M. neoaurum, M. nonchromogenicum, M. novocastrense, M. obuense, M. parqfortuitum, M. paratuberculosis, M. peregrinum, M. phage, M. phlei, M. porcinum, M. poriferae, M. pulveris, M. rhodesiae, M. scrofulaceum, M. senegalense, M. septicum, M. shimoidei, M. simiae, M. smegmatis, M. sphagni, M. szulgai, M. terrae, M. thermoresistibile, M. tokaiense, M. triplex, M. triviale, M. tuberculosis, M. tusciae, M. ulcerans, M. vaccae, M. wolinskyi, M. xenopi, Neisseria animalis, N. canis, N. cinerea, N. denitrificans, N. dentiae, N. elongata, N. flava, N. flavescens, N. gonorrhoeae, N. iguanae, N. lactamica, N. macacae, N. meningitidis, N. mucosa, N. ovis, N. perflava, N. pharyngis var. flava, N. polysaccharea, N. sicca, N. subflava, N. weaveri, Pseudomonas aeruginosa, P. alcaligenes, P. chlororaphis, P. fluorescens, P. luteola, P. mendocina, P. monteilii, P. oryzihabitans, P. pertocinogena, P. pseudalcaligenes, P. putida, P. stutzeri, Salmonella bacteriophage, S. bongori, S. choleraesuis, S. enterica, S. enteritidis, S. paratyphi, S. typhi, S. typhimurium, S. typhimurium, S. typhimurium, S. typhimurium bacteriophage, Shigella boydii, S. dysenteriae, S. flexneri, S. sonnei, Staphylococcus arlettae, S. aureus, S. auricularis, S. bacteriophage, S. capitis, S. caprae, S. carnosus, S. caseolyticus, S. chromogenes, S. cohnii, S. delphini, S. epidermidis, S. equorum, S. felis, S. fleurettii, S. gallinarum, S. haemolyticus, S. hominis, S. hyicus, S. intermedius, S. kloosii, S. lentus, S. lugdunensis, S. lutrae, S. muscae, S. mutans, S. pasteuri, S. phage, S. piscifermentans, S. pulvereri, S. saccharolyticus, S. saprophyticus, S. schleiferi, S. sciuri, S. simulans, S. succinus, S. vitulinus, S. warneri, S. xylosus, Ureaplasma urealyticum, Yersinia aldovae, Y. bercovieri, Y. enterocolitica, Y. frederiksenii, Y. intermedia, Y. kristensenii, Y. mollaretii, Y. pestis, Y. philomiragia, Y. pseudotuberculosis, Y. rohdei, or Y. ruckeri.

31. The method of claim 26, wherein the ribosomes used for screening are derived or purified from obligate intracellular bacteria comprising: Anaplasma bovis, A. caudatum, A. centrale, A. marginale A. ovis, A. phagocytophila, A. platys, Bartonella bacilliformis, B. clarridgeiae, B. elizabethae, B. henselae, B. henselae phage, B. quintana, B. taylorii, B. vinsonii, Borrelia afzelii, B. andersonii, B. anserina, B. bissettii, B. burgdorferi, B. crocidurae, B. garinii, B. hermsii, B. japonica, B. miyamotoi, B. parkeri, B. recurrentis, B. turdi, B. turicatae, B. valaisiana, Brucella abortus, B. melitensis, Chlamydia pneumoniae, C. psittaci, C. trachomatis, Cowdria ruminantium, Coxiella burnetii, Ehrlichia canis, E. chaffeensis, E. equi, E. ewingii, E. muris, E. phagocytophila, E. platys, E. risticii, E. ruminantium, E. sennetsu, Haemobartonella canis, H. felis, H. muris, Mycoplasma arthriditis, M. buccale, M. faucium, M. fermentans, M. genitalium, M. hominis, M. laidlawii, M. lipophilum, M. orale, M. penetrans, M. pirum, M. pneumoniae, M. salivarium, M. spermatophilum, Rickettsia australis, R. conorii, R. felis, R. helvetica, R. japonica, R. massiliae, R. montanensis, R. peacockii, R. prowazekii, R. rhipicephali, R. rickettsii, R. sibirica, or R. typh.

32. The method of claim 26, wherein the ribosomes used for screening are derived or purified from a fungal species.

33. The method of claim 32, wherein the ribosomes used for screening are derived or purified from Candida Candida aaseri, C. acidothermophilum, C. acutus, C. albicans, C. anatomiae, C. apis, C. apis var. galacta, C. atlantica, C. atmospherica, C. auringiensis, C. bertae, C. berthtae var. chiloensis, C. berthetii, C. blankii, C. boidinii, C. boleticola, C. bombi, C. bombicola, C. buinensis, C. butyri, C. cacaoi, C. cantarellii, C. cariosilignicola, C. castellii, C. castrensis, C. catenulata, C. chilensis, C. chiropterorum, C. coipomensis, C. dendronema, C. deserticola, C. diddensiae, C. diversa, C. entomaea, C. entomophila, C. ergatensis, C. ernobii, C. ethanolica, C. ethanothermophilum, C. famata, C. fluviotilis, C. fragariorum, C. fragicola, C. friedrichii, C. fructus, C. geochares, C. glabrata, C. glaebosa, C. gropengiesseri, C. guilliermondii, C. guilliermondii var. galactosa, C. guilliermondii var. soya, C. haemulonii, C. halophila/C. versatilis, C. holmii, C. humilis, C. hydrocarbofumarica, C. inconspicua, C. insectalens, C. insectamans, C. intermedia, C. javanica, C. kefyr, C. krissii, C. krusei, C. krusoides, C. lambica, C. lusitaniae, C. magnoliae, C. maltosa, C. mamillae, C. maris, C. maritima, C. melibiosica, C. melinii, C. methylica, C. milleri, C. mogii, C. molischiana, C. montana, C. multis-gemmis, C. musae, C. naeodendra, C. nemodendra, C. nitratophila, C. norvegensis, C. norvegica, C. oleophila, C. oregonensis, C. osornensis, C. paludigena, C. parapsilosis, C. pararugosa, C. periphelosum, C. petrohuensis, C. petrophilum, C. philyla, C. pignaliae, C. pintolopesii var. pintolopesii, C. pintolopesii var. slooffiae, C. pinus, C. polymorpha, C. populi, C. pseudointermedia, C. quercitrasa, C. railenensis, C. rhagii, C. rugopelliculosa, C. rugosa, C. sake, C. salmanticensis, C. savonica, C. sequanensis, C. shehatae, C. silvae, C. silvicultrix, C. solani, C. sonorensis, C. sorbophila, C. spandovensis, C. sphaerica, C. stellata, C. succiphila, C. tenuis, C. terebra, C. tropicalis, C. utilis, C. valida, C. vanderwaltii, C. vartiovaarai, C. veronae, C. vini, C. wickerhamii, C. xestobii, C. zeylanoides, or Histoplasma capsulatum.

34. The method of claim 26 wherein the ribosomes used for screening are derived or purified from a protozoal species.

35. The method of claim 34, wherein the ribosomes used for screening are derived or purified from Brachiola vesicularum, B. connori, Encephalitozoon cuniculi, E. hellem, E. intestinalis, Enterocytozoon bieneusi, Leishmania aethiopica, L. amazonensis, L. braziliensis, L. chagasi, L. donovani, L. donovani chagasi, L. donovani donovani, L. donovani infantum, L. enriettii, L. guyanensis, L. infantum, L. major, L. mexicana, L. panamensis, L. peruviana, L. pifanoi, L. tarentolae, L. tropica, Microsporidium ceylonensis, M. africanum, Nosema connori, N. ocularum, N. algerae, Plasmodium berghei, P. brasilianum, P. chabaudi, P. chabaudi adami, P. chabaudi chabaudi, P. cynomolgi, P. falciparum, P. fragile, P. gallinaceum, P. knowlesi, P. lophurae, P. malariae, P. ovale, P. reichenowi, P. simiovale, P. simium, P. vinckeipetteri, P. vinckei vinckei, P. vivax, P. yoelii, P. yoelii nigeriensis, P. yoelii yoelii, Pleistophora anguillarum, P. hippoglossoideos, P. mirandellae, P. ovariae, P. typicalis, Septata intestinalis, Toxoplasma gondii, Trachipleistophora hominis, T. anthropophthera, Vittaforma corneae, Trypanosoma avium, T. brucei, T. brucei brucei, T. brucei gambiense, T. brucei rhodesiense, T. cobitis, T. congolense, T. cruzi, T. cyclops, T. equiperdum, T. evansi, T. dionisii, T. godfreyi, T. grayi, T. lewisi, T. mega, T. microti, T. pestanai, T. rangeli, T. rotatorium, T. simiae, T. theileri, T. varani, T. vespertilionis, or T. vivax.

36. The method of claim 26, further comprising a linker that couples the bacterial ribosome ligand and the fluorophore, the linker comprising a carbon chain having 0 to 16 carbons.

37. The method of claim 36, wherein the carbon chain is interrupted by 1 to 6 heteroatoms, functional groups, carbocycles and heterocycles, or by 1 to 6 substituents.

38. The method of claim 26, wherein the antibiotic comprises a 14-membered ring macrolide, a 15-membered ring macrolide, a 16-membered ring macrolide, a tetracycline, an aminoglycoside, an oxazolidinone, clindamycin, puromycin, chloramphenicol, spectinomycin, streptomycin, amikacin, or a pleuromutilin.

39. The method of claim 26, wherein the fluorophore comprises BODIPY, Cy3B, fluorescein, rhodamine, or dipyrrinone.

40. The method of claim 26, wherein the antibiotic comprises a 14-, 15- or 16-membered ring macrolide; the fluorophore selected from a BODIPY, BODIPY. FL, BODIPY. TMR, or Cy3B; and the antibiotic and fluorophore are coupled together by a ligand comprising a carbon chain having 0 to 16 carbons.

41. The method of claim 40, wherein the carbon chain is interrupted by 1 to 6 heteroatoms, functional groups, carbocycles and heterocycles, or 1 to 6 substituents.

42. The method of claim 26, wherein the fluorescent probe comprises: 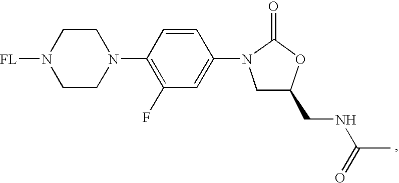
wherein,
FL is the fluorophore comprising:

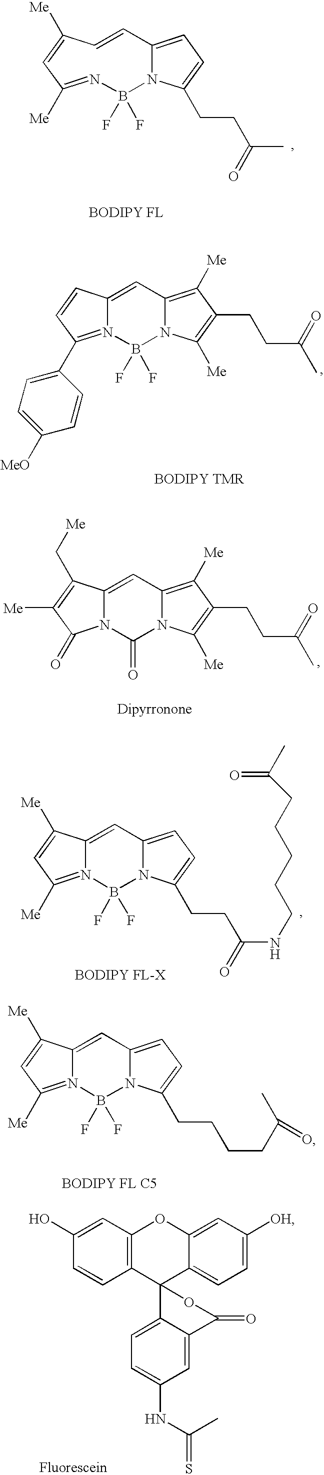

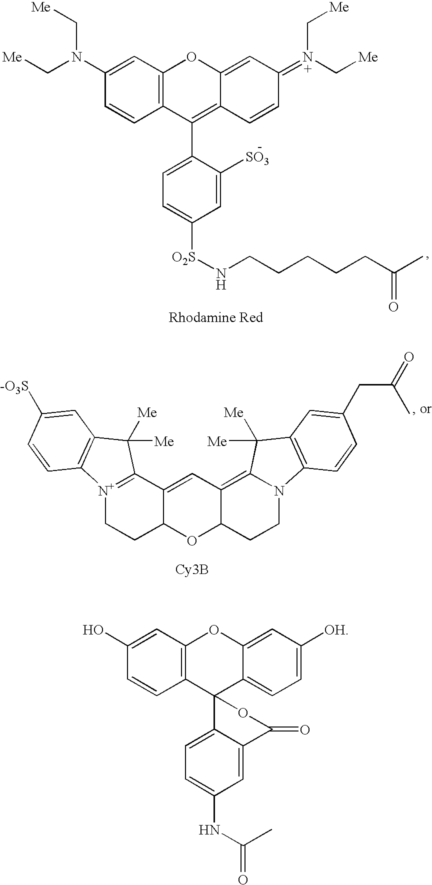

43. The method of claim 26, wherein the fluorescent probe comprises: 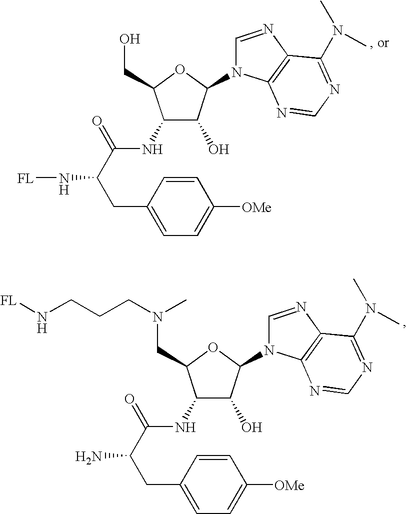
wherein,
FL is the fluorophore comprising:

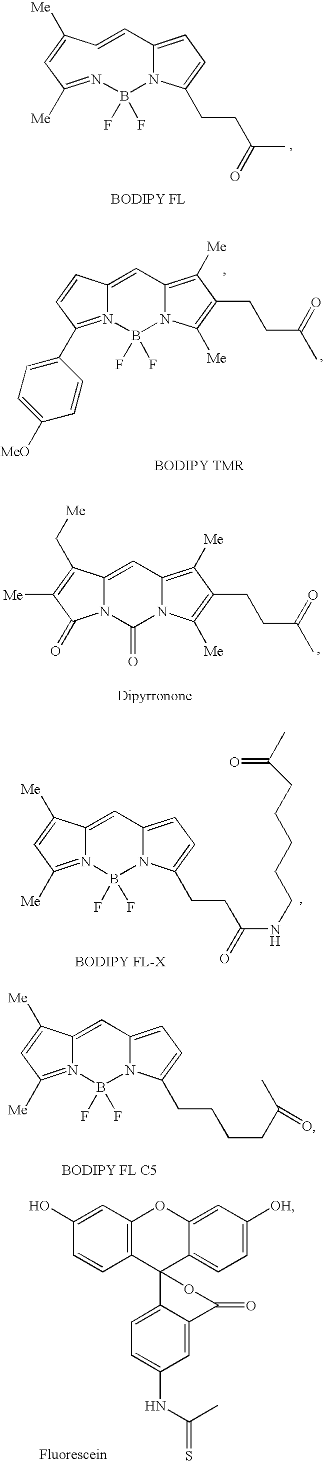

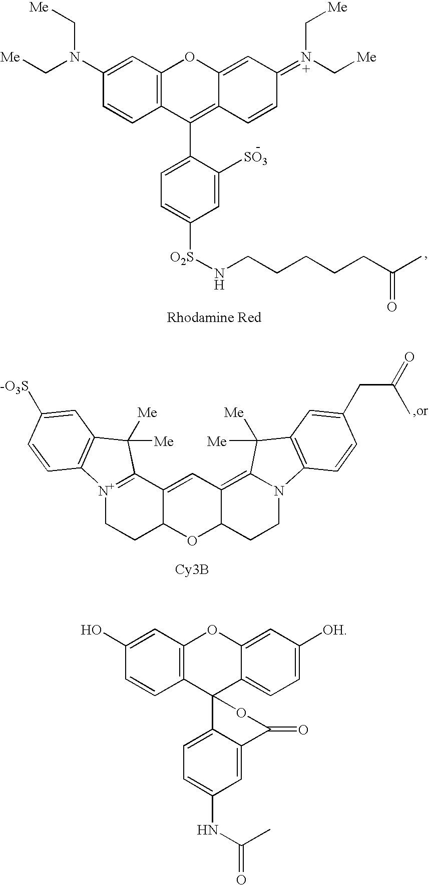

44. The method of claim 26, wherein the fluorescent probe comprises: 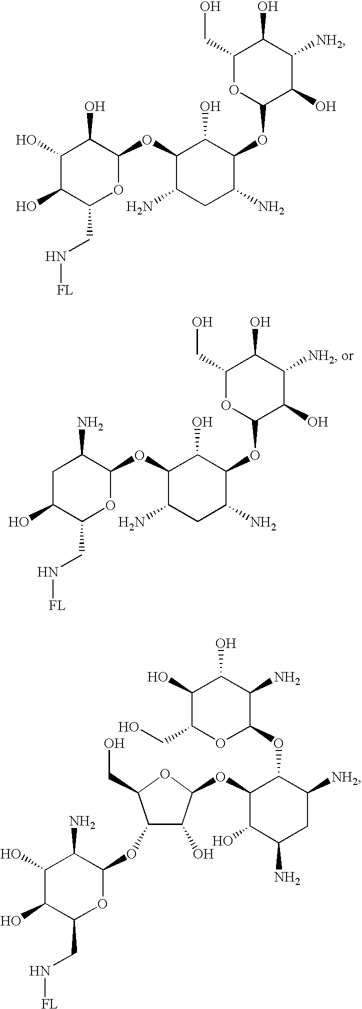
wherein,
FL is the fluorophore comprising:

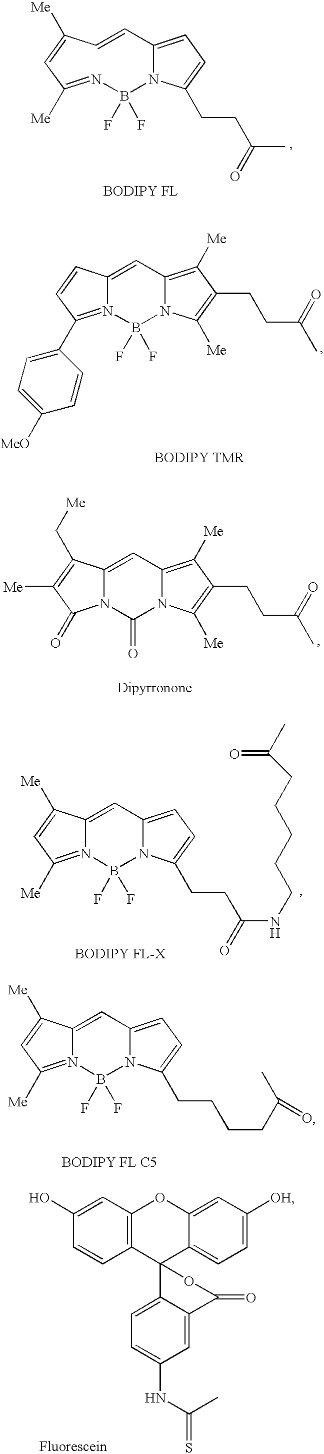

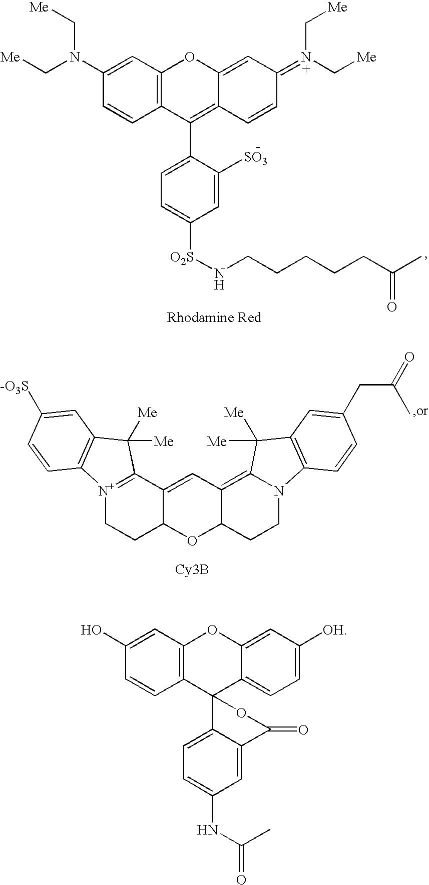

45. The method of claim 26, wherein the fluorescent probe comprises: 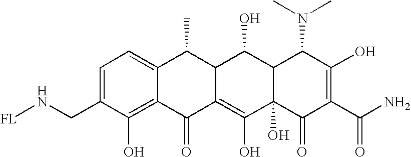
wherein,
FL is the fluorophore comprising:

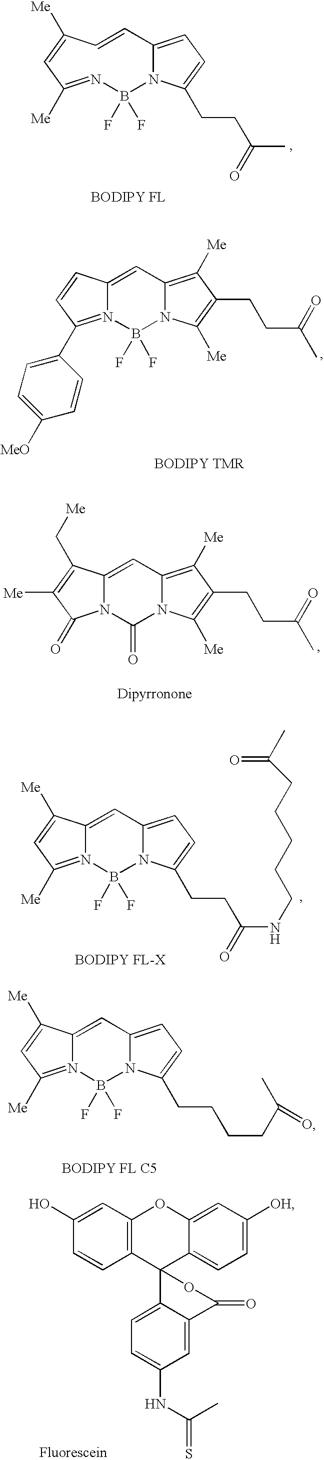

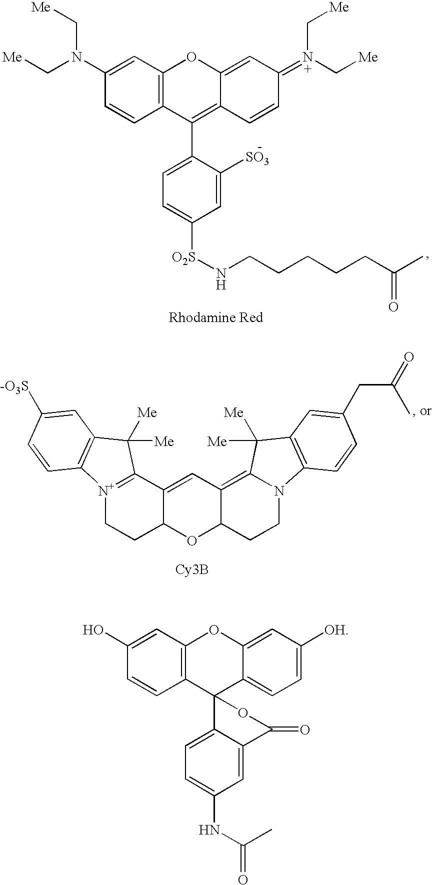

46. The method of claim 26, wherein the fluorescent probe comprises: 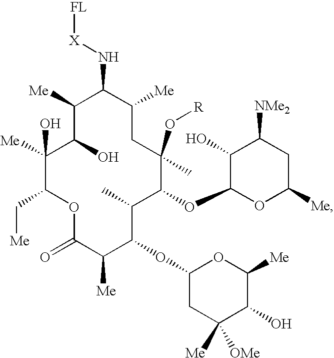
wherein,
FL is the fluorophore comprising: 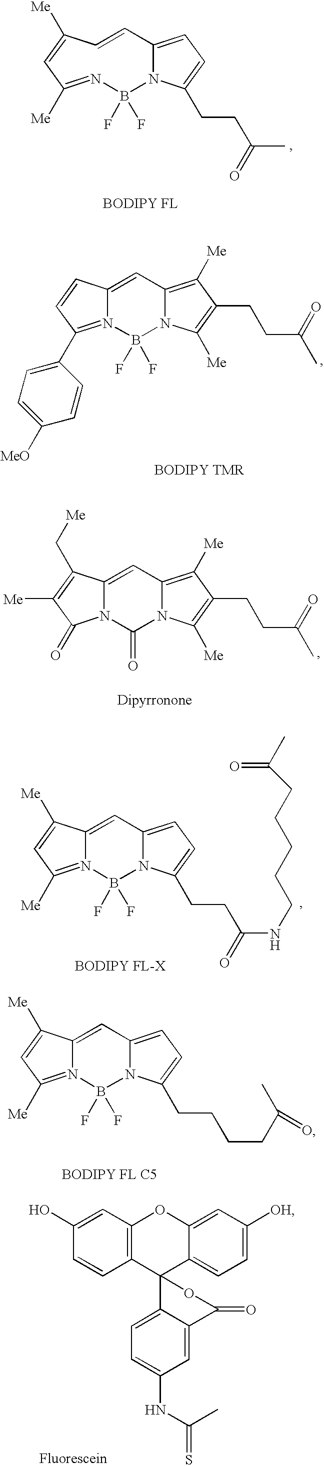 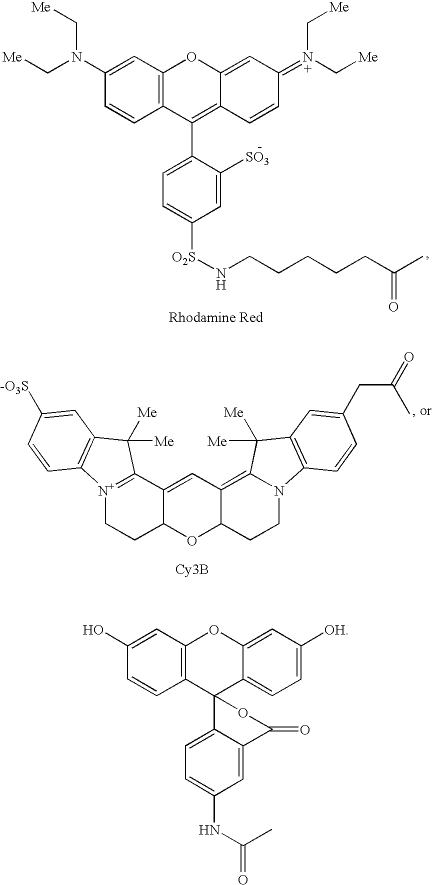
X is none, —(CH₂)ₙNH—, —C(O)—(CH₂)ₙ—NH,— or —C(O)—NH—(CH₂)ₙ—N—, wherein n is a number between 2 and 6; and
R is H or a low alkyl group.

47. The method of claim 26, wherein the fluorescent probe comprises: 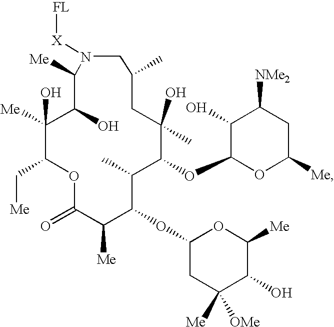
wherein,
FL is the fluorophore comprising: 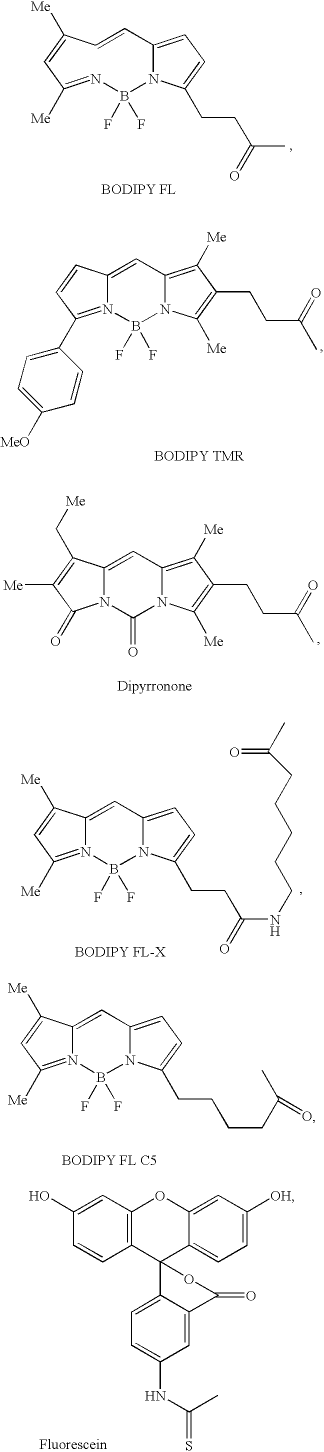 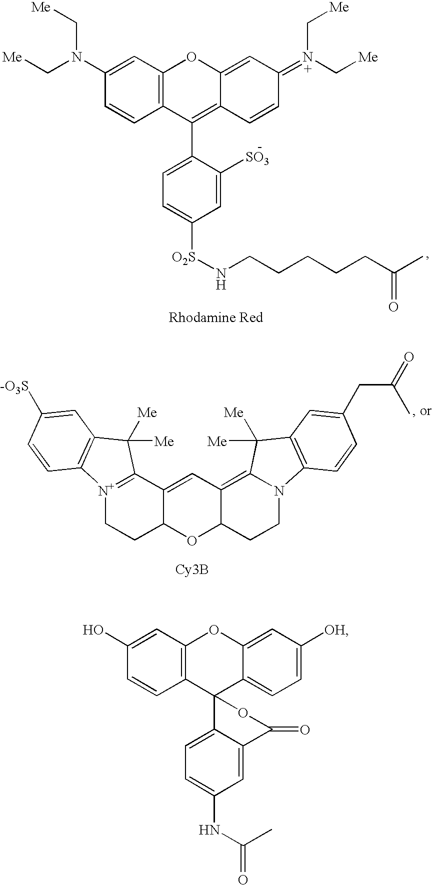
X is none; —(CH₂)ₙNH—; —C(O)—(CH₂)ₙ—NH—; or —C(O)—NH—(CH₂)ₙ—NH—, wherein n is a number between 2 and 6.

48. The method of claim 26, wherein the fluorescent probe comprises: 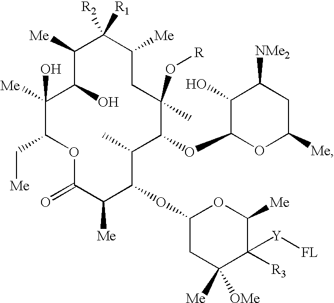
wherein,
FL is the fluorophore comprising: 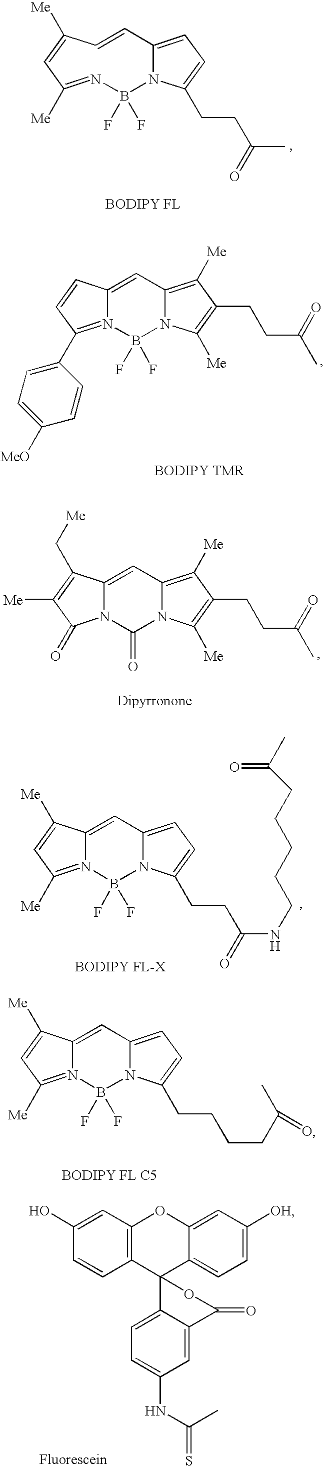 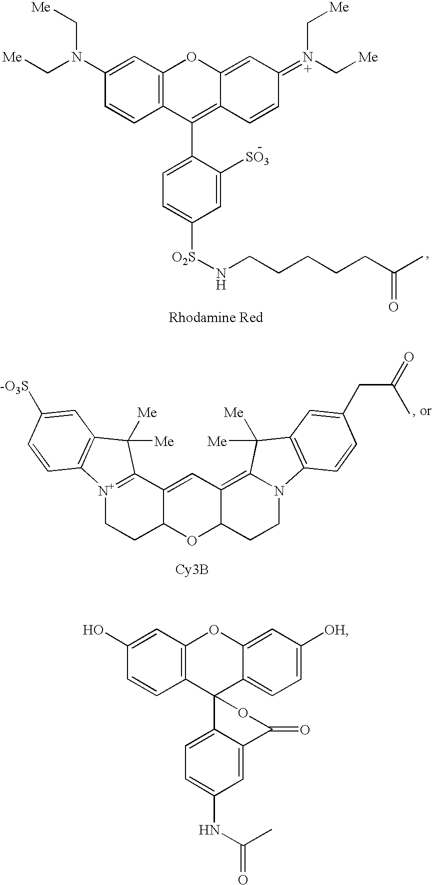
Y comprises: —NH—(CH₂)ₙ—NH—; —NH—C(O)—(CH₂)ₙ—NH—; —NH—C(O)—NH—(CH₂)ₙ—NH—; —O—C(O)—NH—(CH₂)ₙ—NH—; —CH₂—NH—(CH₂)ₙ—NH—; —CH₂—NH—C(O)—(CH₂)ₙ—NH—; or —CH₂—NH—C(O)—NH—(CH₂)ₙ—NH—, wherein n is a number between 2 and 6;
R is H or C1-C6alkyl;
one of R₁ or R₂ is H and the other is selected from: —NR^{a}R^{b}, —OH, or R₁ and R₂ together to form ═O; R^{a} and R^{b} are independently selected from groups consisting of: C₁-C₆ alkyl, —C(O)R^{c}, —C(O)OR^{C}, —C(O)NR^{d}R^{e}, or R^{a} and R^{b} together to form a 3-8 membered heterocycle ring with 1-3 heteroatoms in the ring, optionally substituted with 1-3 substituents; R^{c} is selected from C₁-C₆ alkyl, substituted C₁-C₆ alkyl, aryl, substituted aryl, heteroaryl, and substituted heteroaryl; R^{d} and R^{e} are C₁-C₆ alkyl, aryl, heteroaryl, substituted heteroaryl, or R^{d} and R^{e} together to form a 3-8 membered heterocycle ring; and
R₃ comprises —H or —OH.

49. The method of claim 26, wherein the fluorescent probe comprises: 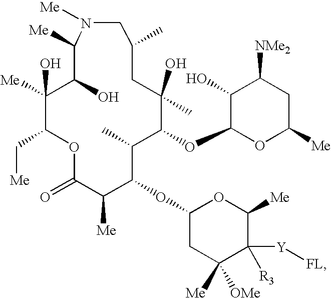
wherein,
FL is the fluorophore comprising: 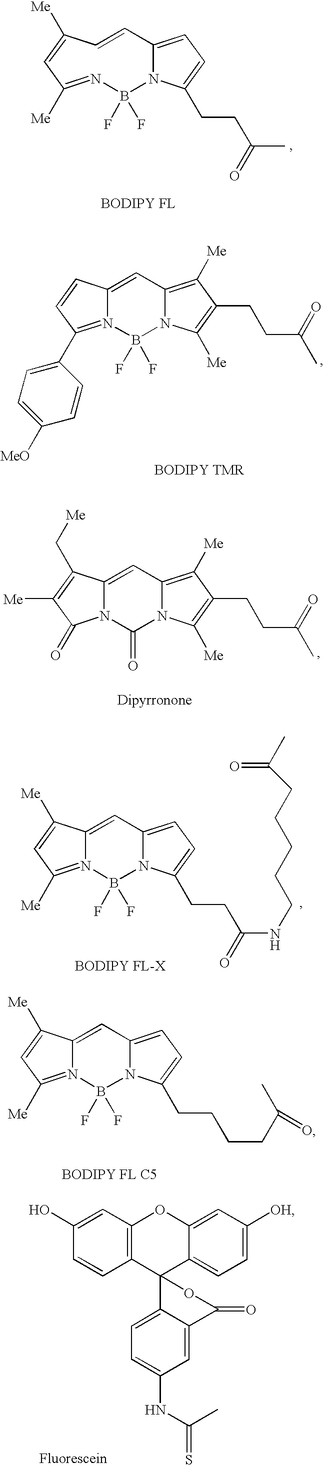 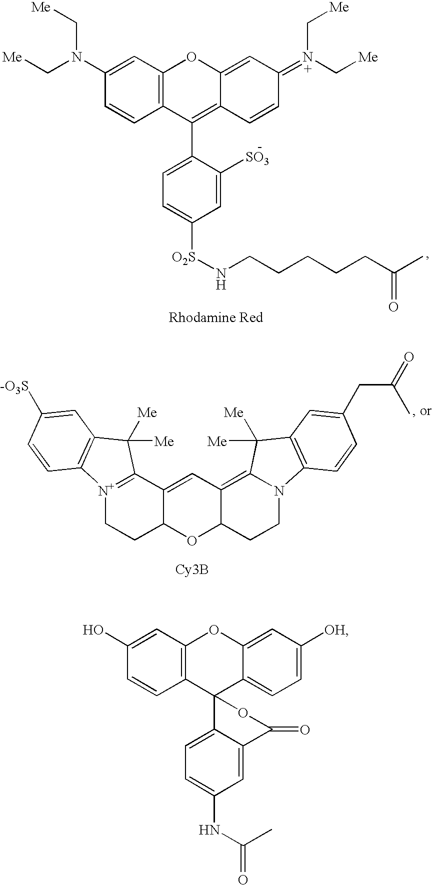
Y comprises: —NH—(CH₂)ₙ—NH—; —NH—C(O)—(CH₂)ₙ—NH—; —NH—C(O)—NH—(CH₂)ₙ—NH—; —O—C(O)—NH—(CH₂)ₙ—NH—; —CH₂—NH—(CH₂)ₙ—NH—; —CH₂—NH—C(O)—(CH₂)ₙ—NH—; or —CH₂—NH—C(O)—NH—(CH₂)ₙ—NH—, wherein n is a number between 2 and 6; and
R₃ comprises —H or —OH.

50. The method of claim 26, wherein the fluorescent probe comprises: 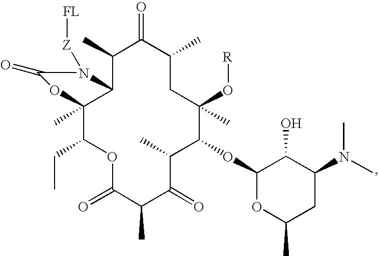
wherein,
FL is the fluorophore comprising: 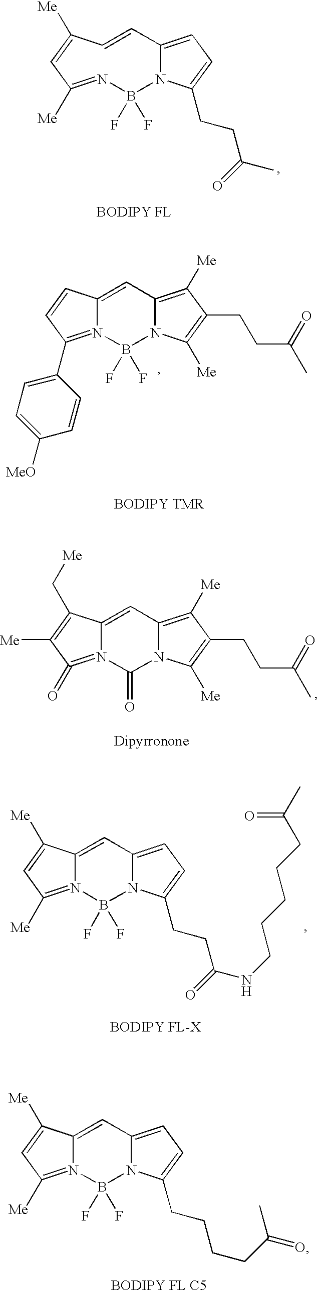 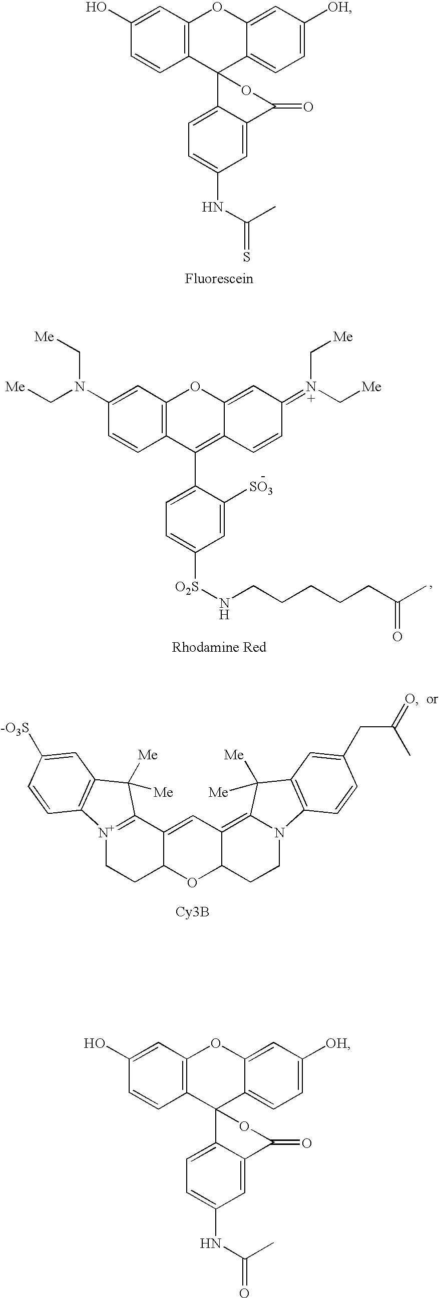
R is H or a low alkyl group;
Z is -A-(CH₂)ₙ—NH—, wherein n is a number between 2 and 6, A is absence, —NH—, or —O—.

51. The method of claim 26, wherein the fluorescent probe comprising: 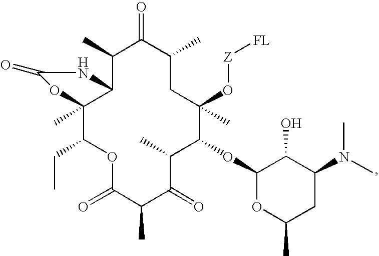
wherein,
FL is the fluorophore comprising: 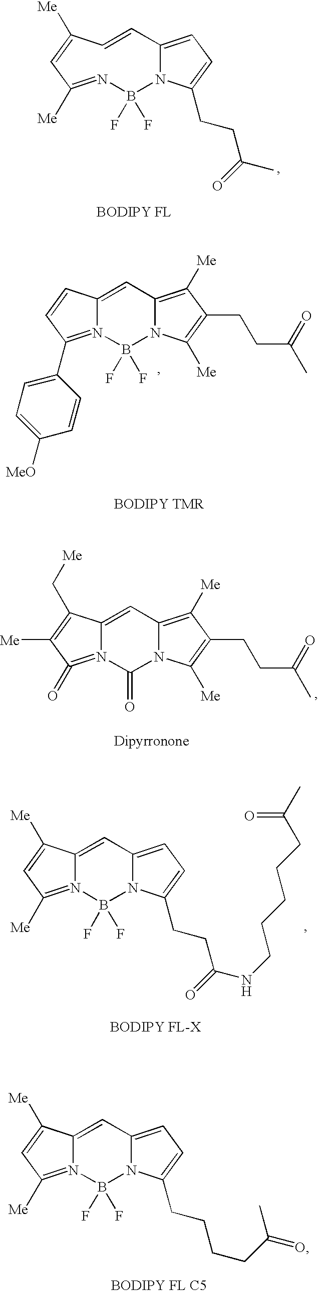 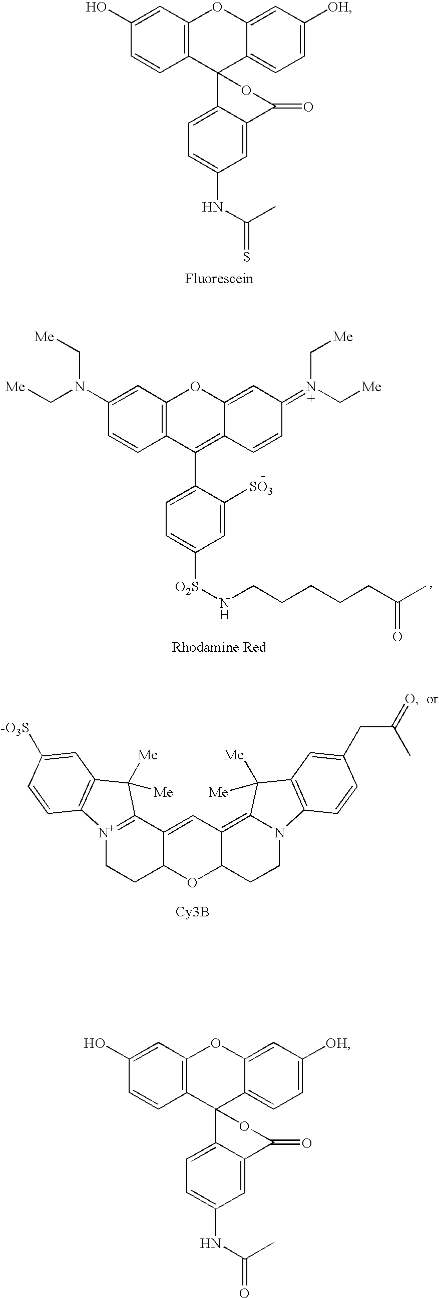
Z is —(CH₂)ₙNH—, wherein n is a number between 2 and 6.

52. The method of claim 26, wherein the fluorescent probe comprises: 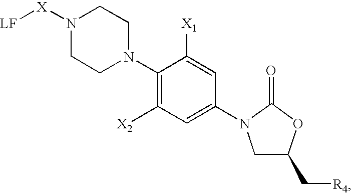
wherein,
FL is the fluorophore comprising: 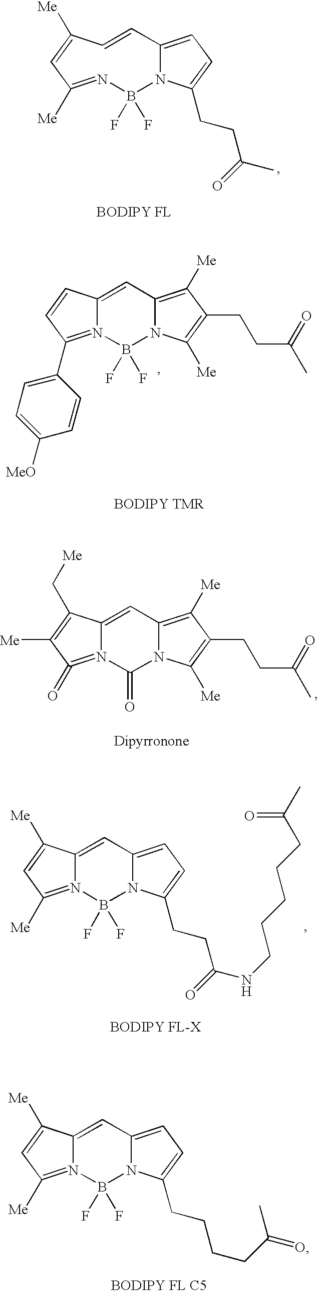 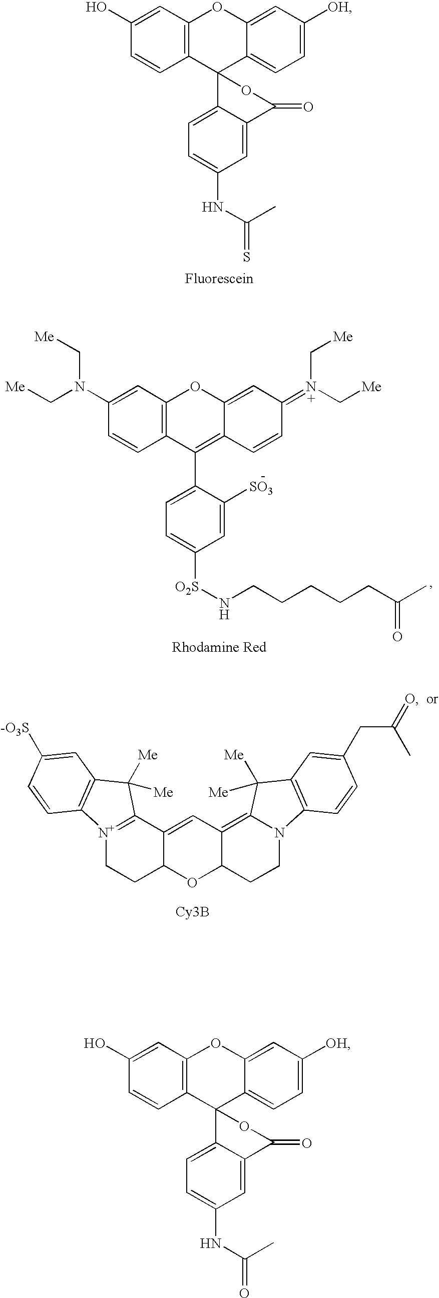
X is none; —(CH₂)ₙNH—; —C(O)—(CH₂)ₙ—NH—; or —C(O)—NH—(CH₂)ₙ—NH—, wherein n is a number between 2 and 6;
X₁ and X₂ are independently —H or —F; and
R₄ comprises:

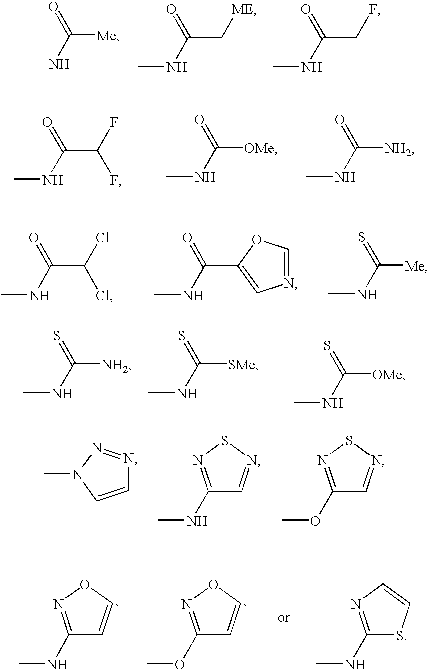

53. The method of claim 26, wherein the fluorescent probe comprises: 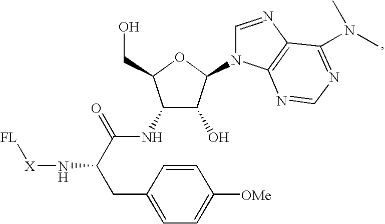
wherein,
FL is the fluorophore comprising: 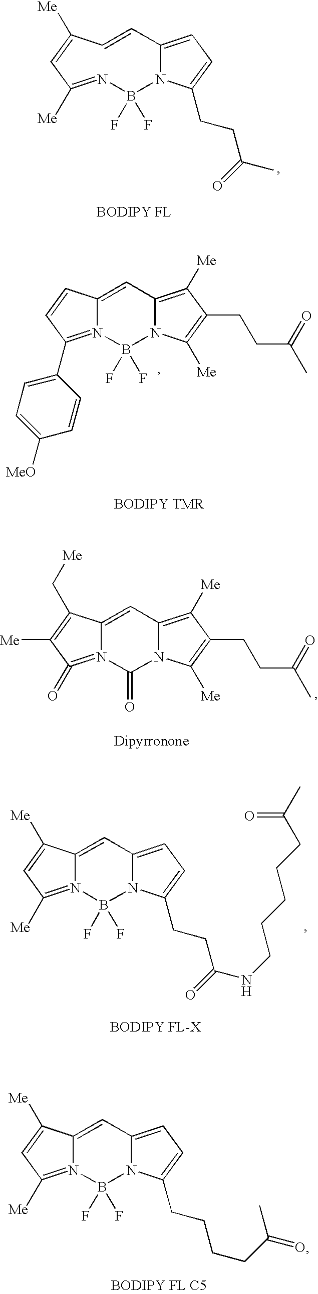 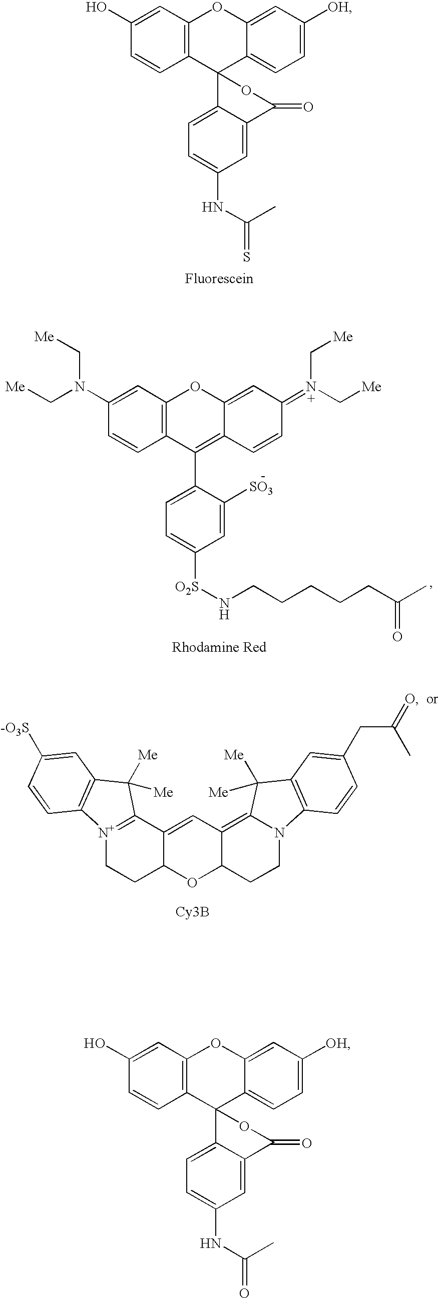
X is none; —(CH₂)ₙNH—; —C(O)—(CH₂)ₙ—NH—; or —C(O)—NH—(CH₂)ₙ—NH—, wherein n is a number between 2 and 6.

54. The method of claim 26, wherein the fluorescent probe comprises: 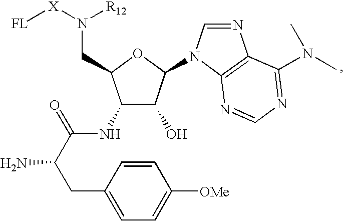
wherein,
FL is the fluorophore comprising: 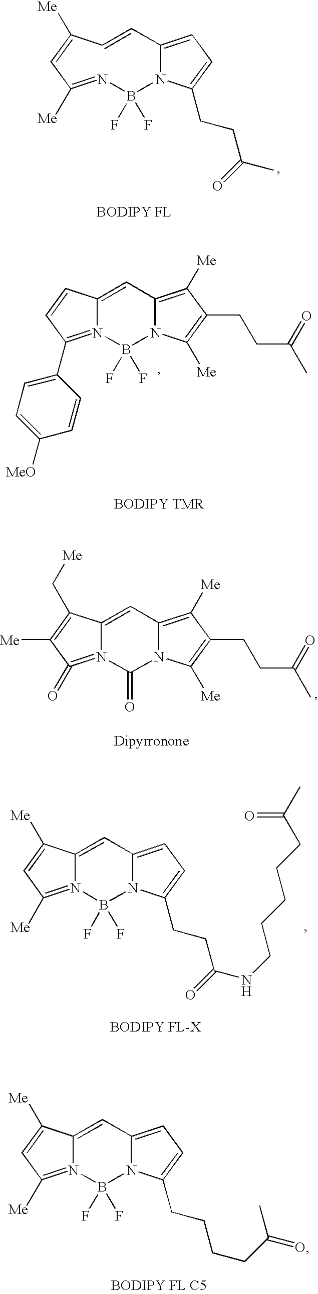 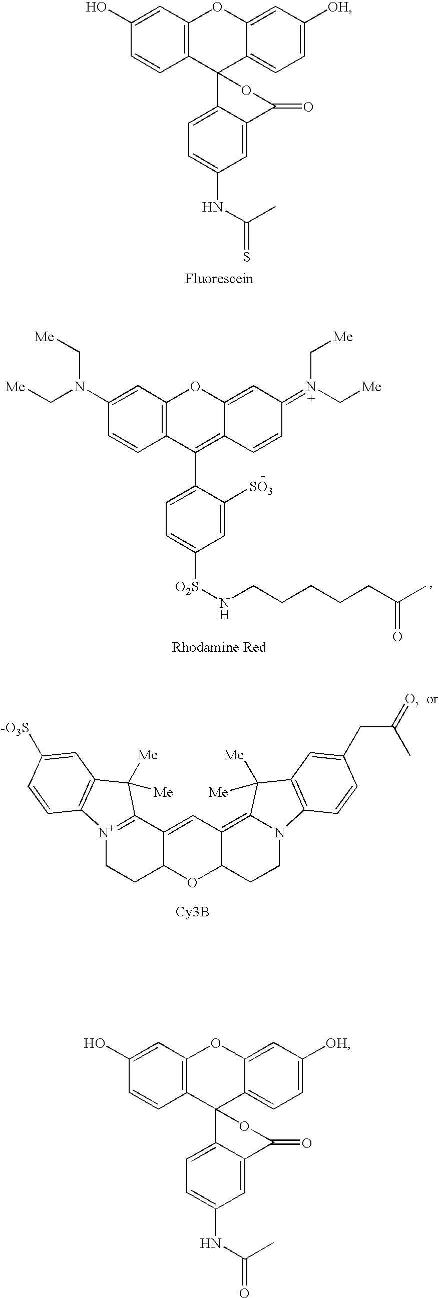
X is none; —(CH₂)ₙNH—; —C(O)—(CH₂)ₙ—NH—; or —C(O)—NH—(CH₂)ₙ—NH—, wherein n is a number between 2 and 6; and
R₁₂ is H or low alkyl.

55. The method of claim 26, wherein the fluorescent probe comprises: 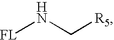
wherein
FL is the fluorophore comprising: 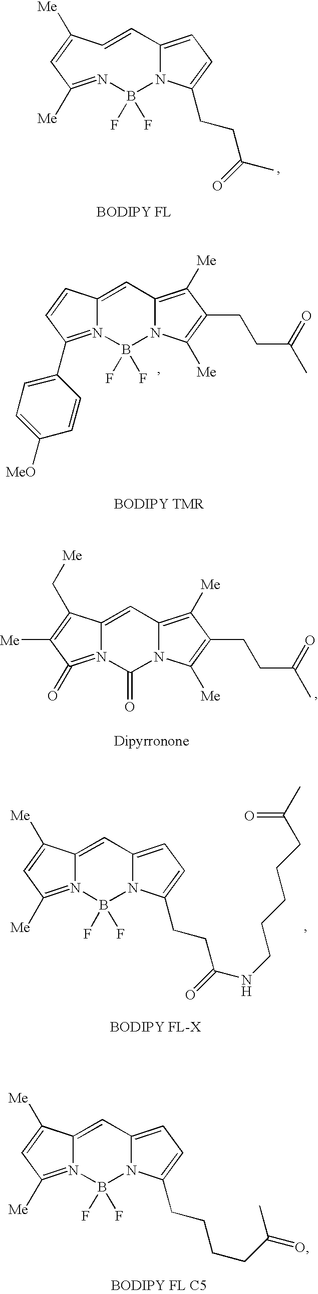 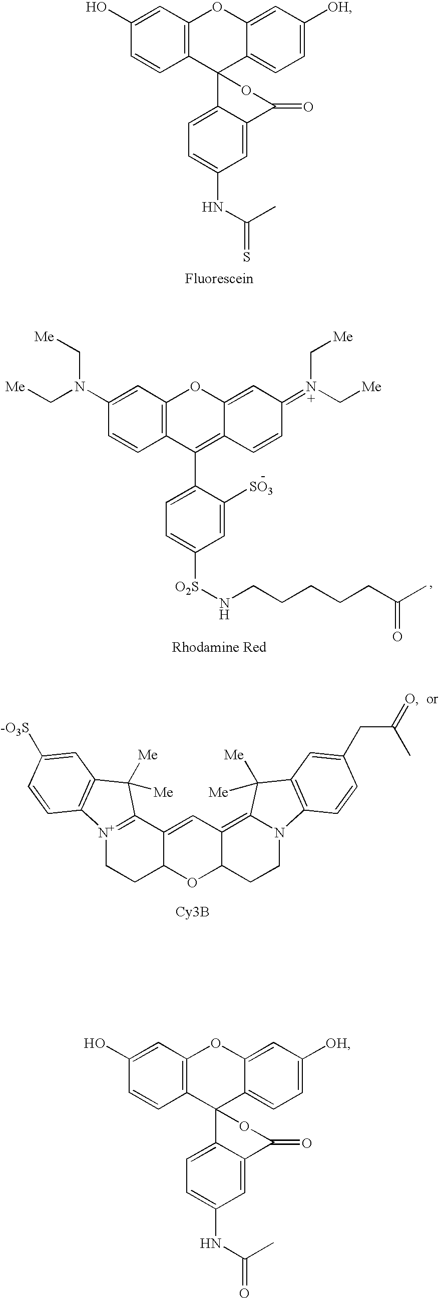
R₅ comprises:

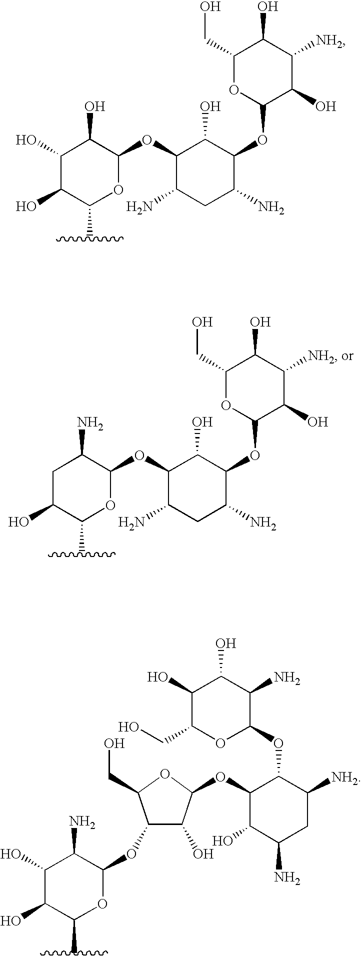

56. The method of claim 26, wherein the fluorescent probe comprises:

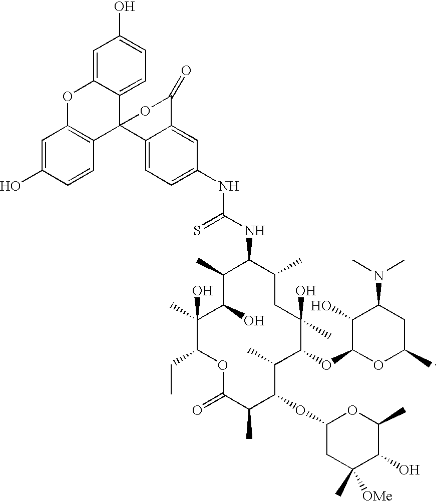

57. The method of claim 26, wherein the fluorescent probe comprises:

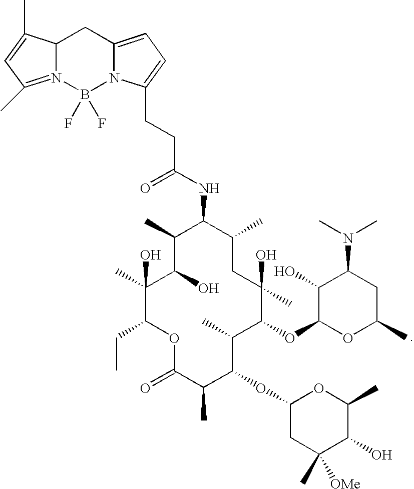

58. The method of claim 26, wherein the fluorescent probe comprises:

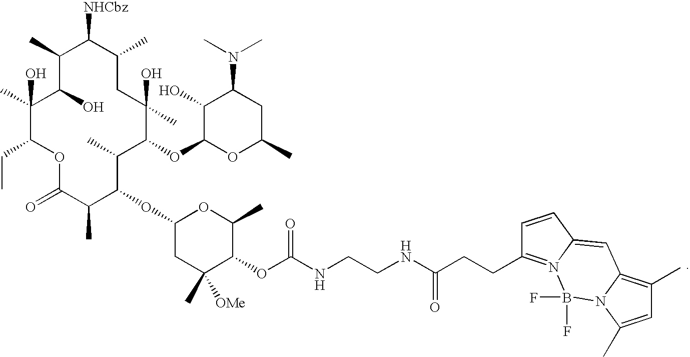

59. The method of claim 26, wherein the fluorescent probe comprises: [chemical structure]
